(19) United States
(12) Patent Application Publication
(10) Pub. No.: US 2026/0331288 A1
(43) Pub. Date: Oct. 22, 2026

(54) HEALTH MANAGEMENT

(71) Applicant: Bao Tran, SARATOGA, CA (US)

(72) Inventor: Bao Tran, SARATOGA, CA (US)

(21) Appl. No.: 19/703,001

(22) Filed: Jun. 9, 2026

Related U.S. Application Data
(62) Division of application No. 18/228,891, filed on Aug. 1, 2023, now abandoned.

(51) Int. Cl. G16H 50/20; G16H 20/30; G16H 20/70; G16H 40/67

(52) U.S. Cl. CPC G16H 50/20; G16H 20/30; G16H 20/70; G16H 40/67

(57) ABSTRACT

Systems and methods are disclosed for health management, comprising: (a) determining one or more factors capable of altering a medical condition of a user; (b) prioritizing the one or more factors using behavioral coaching techniques, comprising generating sub-goal recommendations for each prioritized factor; (c) for each prioritized factor, determining one or more personalized sub-goals dynamically adjusted based on the user's health trajectory; (d) periodically collecting health or fitness data from a wearable device, comprising at least one of blood pressure data, HRV data, SpO2 data, skin temperature data, or physical activity data; (e) analyzing the collected data using ML algorithms to establish a personalized physiological baseline, detect deviations, and identify individual behavior patterns and sub-goal progress; and (f) iteratively generating personalized behavioral interventions delivered via voice coaching, visual alerts, or goal adjustment notifications, adapted in real time.

This application is related to co-pending application Ser. No. 16/743,539 filed Jan. 15, 2020, Ser. No. 17/731,013 filed Apr. 27, 2022, Ser. No. 17/979,756 filed Nov. 2, 2022, Ser. No. 18/115,365 filed Feb. 28, 2023, and Ser. No. 18/218,048 filed Jul. 4, 2023, and 18228991, the contents of which are incorporated by reference.

### BACKGROUND

According to the CDC, in 2021, the Leading Causes of Death for the US include heart disease: 695,547; Cancer: 605,213; COVID-19:416,893; Accidents (unintentional injuries): 224,935; Stroke (cerebrovascular diseases): 162,890; Chronic lower respiratory diseases: 142,342; Alzheimer's disease: 119,399; and Diabetes: 103,294. Solutions are needed to reduce these causes of death.

### SUMMARY OF THE INVENTION

Systems and methods are disclosed to provide health or fitness method for a user by determining one or more factors capable of altering a medical condition; prioritizing the one or more factors for altering the medical condition with cognitive behavioral therapy (CBT); for each prioritized factor, determining one or more sub-goals to improve a user health; periodically collecting heath or fitness data including blood pressure or glucose data from a wearable device, and analyzing the collected data to determine progress on the one or more sub-goals; and iteratively updating the user with progress or result based on the collected data.

The combination of Cognitive Behavioral Therapy (CBT) with wearable devices offers several advantages:
Personalized Interventions: The wearable device provides real-time data on the user's physiological and behavioral patterns. By combining this data with CBT techniques, interventions can be tailored to each individual's specific needs and challenges.
Continuous Monitoring: Wearable devices can continuously monitor various health parameters, such as glucose levels, stress levels, sleep patterns, and physical activity. This enables ongoing assessment and intervention, promoting proactive management of health conditions.
Real-Time Feedback: The wearable device can provide instant feedback to users about their current health status, encouraging behavior change and reinforcing positive habits.
Improved Compliance: CBT interventions delivered through a wearable device are easily accessible and integrated into daily life, increasing user engagement and adherence to the treatment plan.
Early Detection and Prevention: Continuous monitoring allows for the early detection of deviations from normal health patterns, enabling timely intervention and prevention of potential health issues.
Data-Driven Insights: By analyzing data from wearable devices, patterns and trends can be identified, providing valuable insights for healthcare professionals and users alike.
Empowerment and Self-Management: The combination of CBT and wearable devices empowers users to take an active role in managing their health, leading to better self-awareness and self-management.
Longitudinal Tracking: Data collected over time allows for longitudinal tracking of health parameters, facilitating the identification of long-term trends and improvements.
Reduced Healthcare Costs: By promoting proactive health management and preventing complications, the CBT-wearable device combination can potentially reduce healthcare costs associated with managing chronic conditions.
Support for Healthcare Providers: Healthcare providers can gain valuable information from the wearable device data, enabling them to make more informed treatment decisions and offer targeted support to patients.
Remote Monitoring and Telehealth: The combination of CBT and wearable devices allows for remote monitoring of patients, making telehealth and virtual care more effective and accessible.
Gamification and Behavioral Reinforcement: Gamification elements integrated into wearable devices can make health management more engaging and encourage positive behavior change through rewards and incentives.

Overall, the CBT-wearable device combination offers a powerful approach to promote personalized, proactive, and data-driven healthcare, leading to improved patient outcomes and overall well-being.

### BRIEF DESCRIPTION OF THE DRAWINGS

FIG. 1 shows an exemplary method to provide health or fitness using CBT techniques with wearable feedback while FIG. 2 shows an exemplary system for health or fitness for the method of FIG. 1.

FIG. 3 shows exemplary smart watch and mobile device with glucose level feedback.

### DESCRIPTION

Diabetes is a complex metabolic disorder influenced by a combination of genetic, lifestyle, and environmental factors. Here are some of the key factors that can affect diabetes:
Genetics: A family history of diabetes can increase an individual's risk of developing the condition. Certain genes can also predispose individuals to diabetes or affect their insulin production and sensitivity.
Obesity and Weight: Being overweight or obese is a significant risk factor for type 2 diabetes. Excess body fat, particularly in the abdominal area, can lead to insulin resistance and impaired glucose metabolism.
Physical Inactivity: Lack of regular physical activity is associated with an increased risk of developing type 2 diabetes. Exercise helps improve insulin sensitivity and overall metabolic health.
Diet: Unhealthy eating habits, particularly diets high in refined carbohydrates, added sugars, and saturated fats, can contribute to the development of type 2 diabetes. A diet rich in fruits, vegetables, whole grains, and lean proteins can help lower the risk.
Age: The risk of type 2 diabetes increases with age. People over the age of 45 are at a higher risk, particularly if they have other risk factors.
Ethnicity: Some ethnic groups, such as African Americans, Hispanics, Native Americans, and Asians, have a higher predisposition to developing diabetes.
Gestational Diabetes: Women who have had gestational diabetes during pregnancy are at increased risk of developing type 2 diabetes later in life.
Polycystic Ovary Syndrome (PCOS): Women with PCOS are at higher risk of insulin resistance and type 2 diabetes.
High Blood Pressure (Hypertension): Hypertension is often associated with type 2 diabetes and can exacerbate its complications.
Chronic Inflammation: Chronic inflammation in the body can lead to insulin resistance and increase the risk of type 2 diabetes.
Smoking: Smokers have a higher risk of developing type 2 diabetes, and diabetes complications can be more severe in smokers.
Stress: Prolonged stress and inadequate stress management can affect blood glucose levels and worsen diabetes control.

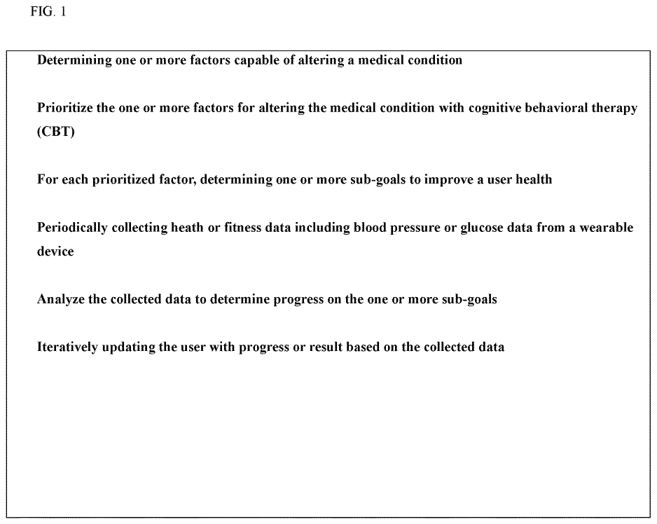

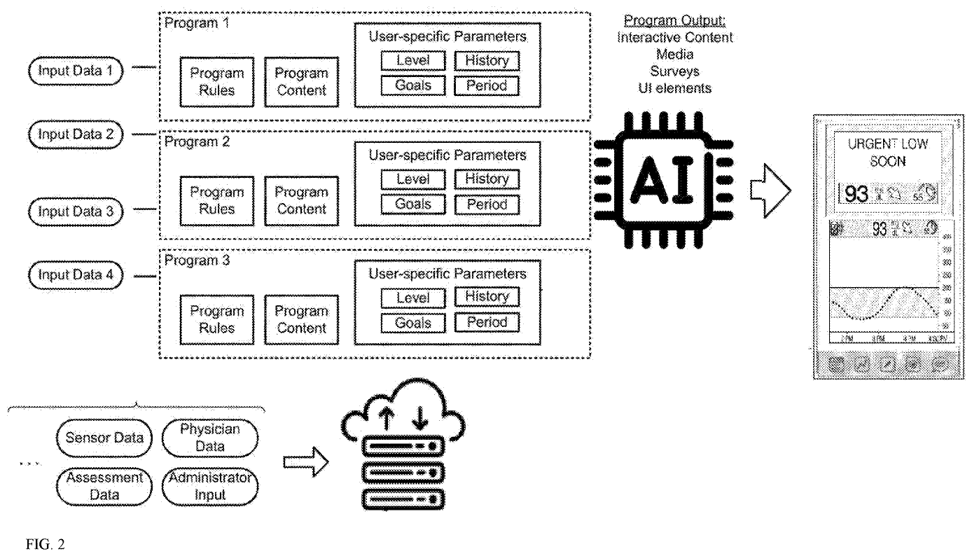

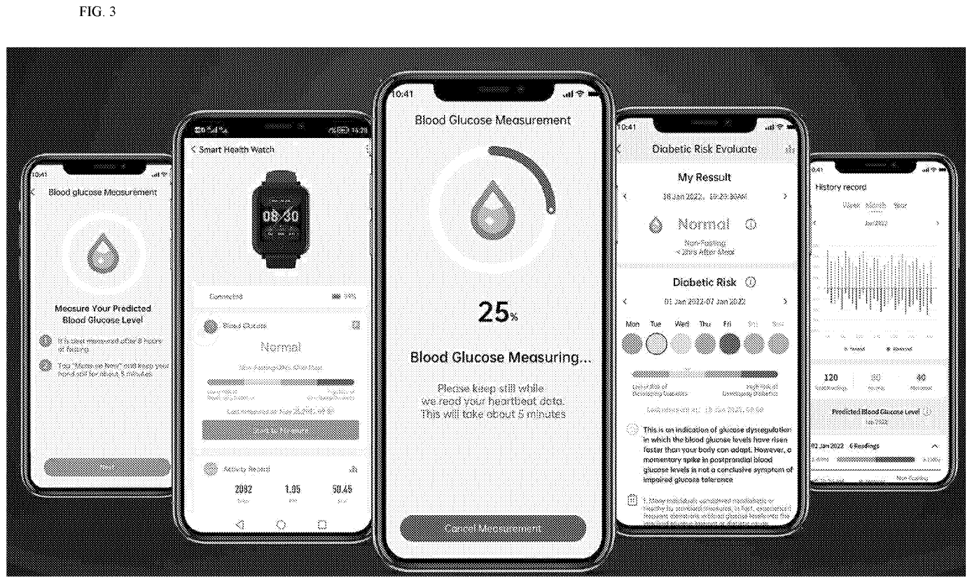

FIG. 1-2 show an exemplary system to address diabetes (and in other embodiments hypertension). method to provide health or fitness using CBT techniques with wearable feedback is run on the system of FIG. 2 to provide health or fitness for the method of FIG. 1. The method includes the following:
determining one or more factors capable of altering a medical condition;
prioritizing the one or more factors for altering the medical condition with cognitive behavioral therapy (CBT);
for each prioritized factor, determining one or more sub-goals to improve a user health;
periodically collecting heath or fitness data including blood pressure or glucose data from a wearable device, and analyzing the collected data to determine progress on the one or more sub-goals; and
iteratively updating the user with progress or result based on the collected data.

Turning now to FIG. 2, The system described in the provided text uses a variety of data structures and algorithms to improve computer performance and provide personalized digital therapeutics programs for users. In FIG. 2, program rules are a set of conditions and triggers that guide the behavior of each digital therapeutics program. These rules are generally applicable to all users of the program but can be filtered and adjusted based on individual user data, historical information, and specific goals. The rules define how the program interacts with the user, what content to present, and when to activate or deactivate certain interventions. Each program may have multiple rules, and only those rules with satisfied conditions and triggers will be executed for a specific user at any given time. For example: Anxiety Program Rule: If the user's stress level is high (based on wearable device data), activate the anxiety program. If the user's stress level reduces significantly for a certain period, deactivate the program or move to a lower intensity level.

Physical Activity Program Rule: If the user's physical activity level drops below a certain threshold (measured by steps taken or minutes of exercise), activate the physical activity program. If the user consistently meets or exceeds daily activity goals, adjust the program level to set more challenging targets.

Nutrition Program Rule: If the user's food or drink intake exceeds certain unhealthy limits (based on user input or wearable device data), activate the nutrition program. If the user maintains a balanced and healthy diet for a period, consider reducing the program's intensity.

Program content represents the source material from which individualized interactions with the user are generated. It includes a wide range of multimedia elements, such as questions, videos, audio segments, images, instructional materials, messages (e.g., encouragement, reminders), games, and other content. The content is specific to each program and aims to address the user's health goals and needs. For example:

Anxiety Program Content: Provide guided breathing exercises, meditation sessions, and cognitive behavioral therapy techniques through video and audio segments. Display supportive messages and reminders to practice relaxation techniques throughout the day.

Physical Activity Program Content: Offer a library of workout routines and fitness videos tailored to the user's fitness level and goals. Provide challenges and rewards for completing daily step goals or exercise sessions.

Nutrition Program Content: Offer personalized meal plans, healthy recipes, and educational content on balanced nutrition. Provide tips and tricks to make healthier food choices and reward users for following the recommended diet.

Each user's care plan includes user-specific parameters for each program, which include their current program level, goals, historical information, and the current period or segment they are in. The combination of program rules and content, along with individualized user parameters, ensures that the digital therapeutics programs adapt to the user's needs, track their progress, and provide tailored interventions to improve their health outcomes.

The system includes:

Dynamic Individualized Care Plans: The system generates dynamic, individualized care plans for each user based on their specific medical conditions, health data, and goals. This is achieved by utilizing various data structures, such as hash tables, to efficiently store and access user-specific parameters for each program.

Program Rules and Content: Each program includes a set of rules and content that are generally applicable to all users but may be filtered and adjusted based on the user's current status and historical information. These rules and content are efficiently stored using data structures like hash tables and linked lists.

Medication Information: The system maintains a medication database that stores information about various pharmaceuticals, their effects, and the relationships between medication effects and different programs. This information is used to determine the relevance of different programs based on the user's current medications and medication history.

Pharmacogenomics Data: User-specific pharmacogenomics data is used to identify relationships between specific genetic features and the effects of medications. This information is used to customize the medication database and provide more personalized interventions.

Program State Setting: The system uses a program state setting module to automatically adjust the levels and states of programs based on the user's input data, program outputs, and other factors. This is achieved by analyzing program outputs, sentiment analysis, and assessing the frequency of program activity.

Population Benchmarking: The system benchmarks each user's progress against population data to motivate users to strive for better health outcomes. This involves comparing user-specific data with aggregated data from similar users to identify effective interventions.

Longitudinal Insights: The system uses longitudinal data to track users' progress over time and identify trends and patterns in their health data. This helps in generating more effective and personalized care plans.

Quadtree or Octree: The system may use quadtree or octree data structures to efficiently organize and manage spatial data, such as location-based information or environmental data.

Overall, the system's efficient use of data structures, data analysis, and dynamic program adjustment allows it to provide scalable and reliable digital therapeutics programs for millions of users, tailored to their unique needs and medical conditions.

LLM (Large Language Model), like GPT-5, can be used to generate interactive content, media, surveys, voice, video, UI elements, and other forms of output to provide an individualized care plan with an interactive user interface. Here's how it can be accomplished:

Natural Language Generation (NLG): LLM can be used for natural language generation, allowing it to create personalized and interactive content for the user. Based on the user's input data, historical information, and goals, LLM can dynamically generate text-based interactions, such as personalized messages, reminders, instructions, and educational content related to the user's health condition and care plan.

Media Content Generation: LLM can also generate various types of media content, including images, videos, and audio segments, based on the user's needs and preferences. For example, it can create personalized exercise videos, meditation sessions, or cooking tutorials that align with the user's fitness level, stress level, or dietary requirements.

Surveys and Questionnaires: LLM can automatically generate personalized surveys and questionnaires to collect specific information from the user. These surveys can be designed to assess the user's progress, gather feedback, and understand the effectiveness of interventions. LLM can adapt the questions based on the user's responses to ensure relevant and targeted data collection.

Voice and Video Interactions: LLM can be integrated with voice and video processing technologies to enable voice-based interactions and video-based coaching sessions. It can provide personalized voice prompts for guided meditation, workout routines, or medication reminders. In video interactions, LLM can dynamically generate real-time coaching sessions with virtual coaches tailored to the user's needs.

User Interface (UI) Elements: LLM can generate interactive UI elements that dynamically adjust based on the user's input and current progress. For example, it can create personalized progress trackers, goal-setting interfaces, and data visualization components to help users monitor their achievements and visualize their health data effectively.

Real-time Personalization: LLM can analyze the user's real-time data from wearable devices, medical records, and other sources to adjust the content and interactions on-the-fly. For instance, it can dynamically update exercise routines based on the user's physical activity levels or modify dietary recommendations based on glucose levels (or blood pressure level).

Behavior Prediction and Gamification: LLM can predict the user's behavior patterns by analyzing historical data and interactions. It can then gamify the care plan by setting challenges, providing rewards, and offering incentives to encourage the user to achieve their health goals.

Adaptation to User Feedback: LLM can respond to user feedback and adapt the care plan accordingly.

If the user reports side effects or preferences, LLM can modify the interventions to better suit the user's needs and preferences.

By leveraging LLM's language generation capabilities and integrating it with other technologies, the system can automatically provide a highly individualized care plan with interactive and engaging user interfaces to support and motivate the user effectively in their health journey.

LLM (Large Language Model) refers to advanced language models like GPT-3 that can understand and generate human-like text based on the input it receives. Behavior prediction, in this context, involves using historical data and user interactions to forecast a user's future actions or behavior patterns. Here's a high-level explanation of how LLM can be used for behavior prediction with pseudo-code:

### Data Collection and Preprocessing:

Gather historical data about the user's interactions, behaviors, and health-related information. Preprocess the data to extract relevant features and create a structured dataset.

### Training the Language Model (LLM):

Train the LLM on the preprocessed dataset to learn patterns and relationships between user inputs and corresponding outcomes.
Fine-tune the LLM on specific health-related tasks or objectives, such as exercise prediction, stress management, or dietary adherence.

### Behavior Prediction Function:

#Pseudo-code for the behavior prediction function
def predict_user_behavior(llm_model, user_input):
#Generate user-specific context for LLM input (combine user_input with historical data)
context=preprocess_user_input(user_input)
   #Use the trained LLM to predict the next behavior or action
predicted_behavior=llm_model.generate_response(context)
   return predicted_behavior

### Real-Time User Interaction and Prediction:

#Pseudo-code for real-time user interaction and behavior prediction
def main( ):
   #Initialize the LLM model and load pre-trained weights
   llm_model=initialize_llm_model( )
      #Retrieve user input (e.g., data from wearable devices, health survey responses)
   user_input=get_user_input( )
      #Use the LLM to predict the next behavior based on the user's input and historical data predicted_behavior=predict_user_behavior (llm_model, user_input)
      #Display the predicted behavior to the user
   display_predicted_behavior(predicted_behavior)

In this pseudo-code, the predict_user_behavior function takes the LLM model and user input as input and returns the predicted behavior. The main function represents the real-time user interaction, where the LLM model is initialized, user input is obtained (e.g., from wearable devices or surveys), and the predicted behavior is displayed back to the user.

The LLM model, having learned from historical data, can provide valuable insights and predictions about the user's future behaviors. For instance, it can predict whether the user is likely to engage in a specific exercise routine, adhere to their dietary plan, or follow stress management techniques. These predictions can then be used to dynamically adjust the care plan and provide targeted interventions and gamified challenges to encourage positive health behaviors and outcomes.

### LLM can be Applied to Behavior Modification:

Understanding User Input: LLM can process natural language input from users, such as text messages, speech, or survey responses, and comprehend the user's specific health concerns, challenges, and goals. This understanding allows the system to identify the areas where behavior modification is needed.

Personalized Feedback and Suggestions: LLM can generate personalized feedback and suggestions based on the user's input and historical data. For example, if a user expresses struggles with adhering to a healthy diet, LLM can provide specific advice, meal planning ideas, and motivational messages to support the user's dietary goals.

Gamification and Interactive Interventions: LLM can create interactive gamified challenges and activities that encourage users to engage in positive health behaviors. It can generate rewards, achievements, and progress tracking mechanisms to enhance user motivation and commitment to behavior modification.

Goal Setting and Progress Monitoring: LLM can help users set realistic and achievable goals based on their health status and preferences. It can track progress over time and provide continuous updates to the user, celebrating successes and offering support in case of setbacks.

Contextual Adaptation: LLM can adapt its responses and interventions based on the user's current context, such as location, time of day, mood, or physical activity level. This contextual adaptation ensures that the behavior modification strategies remain relevant and effective for the user's real-life situations.

Behavior Prediction: Using historical data and machine learning techniques, LLM can predict future behavior patterns of users. By understanding past behaviors, it can anticipate potential challenges or triggers, enabling proactive interventions to prevent negative outcomes and reinforce positive behaviors.

Therapist Support: LLM can serve as a virtual therapist, providing continuous support and guidance to users. It can answer questions, offer coping strategies, and provide empathetic responses to enhance user engagement and emotional well-being.

Continual Learning: LLM can continually learn from user interactions and feedback, improving its ability to deliver more accurate and effective behavior modification interventions over time.

Overall, LLM's natural language processing capabilities, coupled with its ability to understand user context and generate personalized content, make it a powerful tool for behavior modification. By leveraging these features, LLM can support users in making positive health behavior changes and ultimately improve their overall well-being.

LLM (Large Language Model) can be applied to understand and analyze gaming behavior to provide personalized recommendations, improve gaming experiences, and address potential issues related to excessive gaming or addiction. Here's an example of how LLM can be used with pseudo code:
#Pseudo code for LLM-based Gaming Behavior Analysis
#Step 1: User Input
user_input=get_user_input( ) #Get input from the user (e.g., text messages, survey responses)
#Step 2: Behavior Analysis
behavior_analysis_result=LLM_analyze_behavior(user_input) #LLM processes user input and analyzes gaming behavior
#Step 3: Personalized Recommendations
if behavior_analysis_result[‘time_played’]>=‘5 hours’:
   personalized_recommendation=“It seems you are spending a significant amount of time gaming. Consider taking breaks and balancing gaming with other activities.”
elif behavior_analysis_result[‘in-game_purchases’]>=‘10 purchases’:
   personalized_recommendation=“You've made many in-game purchases. Be mindful of your spending and consider setting limits on purchases.”
else:
   personalized_recommendation=“Your gaming behavior looks balanced. Keep up the good work!”
#Step 4: Gaming Experience Enhancement
if behavior_analysis_result[‘preferred_game_genres’]==‘Action’:
   game_suggestion=“Based on your preference for action games, you might enjoy trying ‘Game X’ or ‘Game Y’.”
elif behavior_analysis_result[‘preferred_game_genres’]==‘Simulation’:
   game_suggestion=“If you like simulation games, give ‘Game Z’ a try!”
else:
   game_suggestion=“Explore various game genres to find your perfect match!”
#Step 5: Addressing Excessive Gaming
if behavior_analysis_result[‘time_played’]>=‘8 hours’:
   gaming_addiction_support=“If you feel your gaming is interfering with other aspects of life, consider seeking professional help or setting time limits.”
else:
   gaming_addiction_support=“Keep gaming for fun, but remember to take breaks and prioritize other responsibilities.”
#Step 6: Providing Supportive Messages
supportive_message=LLM_generate_supportive_message(behavior_analysis_result) #LLM generates empathetic and supportive messages
#Step 7: Display Recommendations
display_recommendations(personalized_recommendation, game_suggestion, gaming_addiction_support, supportive_message) #Display recommendations to the user

In this pseudo code, the LLM component is responsible for analyzing user input related to gaming behavior. It can assess factors such as time spent gaming, in-game purchases, preferred game genres, and other relevant data. Based on the analysis, LLM generates personalized recommendations, game suggestions, and supportive messages to enhance the user's gaming experience and address potential concerns.

### Risk Assessment

Next is an exemplary Risk Assessment Survey letter to a patient to scope out potential problems. The survey can be a computer UI or can be an email or a letter that the user can write in the answers and mail to a collection site. The survey letter can be as follows:
Subject: Diabetes Risk Assessment Survey
Dear [Patient's Name],
We hope this message finds you well. As part of our commitment to your overall health and well-being, we are conducting a Diabetes Risk Assessment Survey. This survey aims to identify potential risk factors that may contribute to diabetes development. Your participation in this survey is completely voluntary, and all information provided will be kept confidential. Please take a few minutes to answer the following questions honestly. Your responses will help us better understand your risk profile and enable us to provide appropriate guidance and support.
Genetics: Do you have any immediate family members (parents or siblings) who have been diagnosed with diabetes? (Yes/No)
Obesity and Weight: Are you currently overweight or obese? (Yes/No) If yes, please provide your approximate weight and height for calculation of Body Mass Index (BMI).
Physical Inactivity: How many days per week do you engage in moderate to vigorous physical activity (e.g., brisk walking, jogging, swimming, etc.) for at least 30 minutes?
Diet: How often do you consume foods high in refined carbohydrates, added sugars, and saturated fats (e.g., sugary drinks, fast food, desserts, etc.)?
Age: What is your current age?
Ethnicity: Please indicate your ethnic background, as some ethnic groups have a higher predisposition to diabetes.
Gestational Diabetes: Have you ever been diagnosed with gestational diabetes during a pregnancy? (Yes/No)
Polycystic Ovary Syndrome (PCOS): Are you a woman who has been diagnosed with PCOS? (Yes/No)
High Blood Pressure (Hypertension): Have you been diagnosed with hypertension (high blood pressure)? (Yes/No)
Chronic Inflammation: Do you have any chronic inflammatory conditions? (Yes/No)
Smoking: Do you currently smoke cigarettes or use any tobacco products? (Yes/No)
Stress: How would you rate your overall stress level on a scale of 1 to 10, with 10 being the highest?
Your participation in this survey will be immensely helpful in assessing your risk for diabetes accurately. Please submit your responses at your earliest convenience. If you have any questions or concerns, feel free to reach out to us.
Thank you for your cooperation in this important endeavor to safeguard your health.
Best regards,
[Your Healthcare Provider's Name]
[Medical Facility Name]
[Contact Information]

Based on the user response, further followups can be done. For example, the system can send this followup to point 1 if problematic
Subject: Follow-up on Diabetes Risk Assessment Survey—Genetic Factors
Dear [Patient's Name],
Thank you for participating in our Diabetes Risk Assessment Survey. We appreciate your commitment to understanding and improving your health. We have reviewed your responses, and we would like to discuss the genetic risk factors for diabetes that you mentioned in the survey.
If you indicated that you have immediate family members (parents or siblings) who have been diagnosed with diabetes, it is essential to recognize that genetics can play a significant role in the development of this condition. Having a family history of diabetes may increase your risk of developing the disease.
While genetics can contribute to diabetes risk, it is essential to remember that it is only one of several factors that can influence the development of the condition. Lifestyle factors, such as diet, physical activity, and weight management, also play crucial roles in determining diabetes risk.
Given your family history of diabetes, we recommend taking proactive steps to minimize your risk and promote overall health. Here are some actions you can consider:
Regular Health Check-ups: Schedule regular check-ups with your healthcare provider to monitor your blood glucose levels and overall health.
Healthy Eating Habits: Focus on a balanced diet rich in fruits, vegetables, whole grains, and lean proteins. Limit the intake of sugary and processed foods.
Physical Activity: Engage in regular physical activity to improve insulin sensitivity and maintain a healthy weight.
Maintain a Healthy Weight: If you are overweight, a gradual and sustainable weight loss plan can help reduce your risk of developing diabetes.
Know the Warning Signs: Familiarize yourself with the early warning signs of diabetes, such as increased thirst, frequent urination, fatigue, and unexplained weight loss. If you experience any of these symptoms, please consult your healthcare provider promptly.
Stay Informed: Educate yourself about diabetes and the factors that can influence its development. Knowledge empowers you to make informed decisions about your health.
Please remember that even if you have a family history of diabetes, it does not mean you will definitely develop the condition. Proactive lifestyle changes can significantly reduce your risk and improve your overall well-being.
If you have any questions or concerns or need further guidance, please don't hesitate to reach out to us. We are here to support you on your journey to better health.

### An Exemplary Follow-Up to Point 2 is as Follows:

Subject: Follow-up on Diabetes Risk Assessment Survey-Obesity and Weight
Dear [Patient's Name],
Thank you for participating in our Diabetes Risk Assessment Survey and sharing your responses with us. Your dedication to understanding your health and well-being is commendable.
We noticed that you mentioned being overweight or obese in the survey. We understand that excess body weight can be a challenging issue to address, but we want to emphasize that it is a critical risk factor for diabetes, particularly type 2 diabetes.
Being overweight or obese can lead to insulin resistance, where your body becomes less responsive to the hormone insulin, which regulates blood glucose levels. Insulin resistance can eventually progress to diabetes if left unaddressed.
We want to assure you that we are here to support you in your efforts to improve your health and manage your weight effectively. Here are some steps you can consider taking to reduce your risk of diabetes and improve your overall well-being:
Healthy Eating Habits: Focus on a balanced diet that includes a variety of nutritious foods.
Limit the consumption of sugary drinks, processed foods, and high-calorie snacks.
Portion Control: Be mindful of portion sizes and avoid overeating, even if you are consuming healthy foods.
Regular Physical Activity: Engage in regular exercise or physical activity that you enjoy. It doesn't have to be intense; even moderate activity can have significant health benefits.
Seek Professional Guidance: Consider consulting with a registered dietitian or a healthcare provider who specializes in weight management. They can provide personalized advice and help you create a sustainable plan to achieve your weight goals.
Set Realistic Goals: Set achievable and realistic weight loss goals to avoid frustration and maintain motivation.
Stay Consistent: Consistency is key to achieving lasting results. Focus on forming healthy habits rather than short-term fixes.
Remember, improving your health is a journey, and small steps can lead to significant changes over time. Celebrate your progress along the way, and don't be too hard on yourself if you face challenges. We are here to provide support and guidance throughout your health journey.
If you have any questions or need further assistance, please feel free to reach out to us. We are dedicated to helping you take control of your health and well-being.

### An Exemplary Follow-Up to Point 3 if Problematic:

Subject: Follow-up on Diabetes Risk Assessment Survey-Physical Inactivity
Dear [Patient's Name],
We sincerely appreciate your participation in our Diabetes Risk Assessment Survey and providing us with valuable information about your health. Your commitment to understanding your risk factors is commendable.
In the survey, you mentioned that you engage in physical activity for fewer days per week than recommended for at least 30 minutes. We want to acknowledge that maintaining an active lifestyle can be challenging, especially with busy schedules and other responsibilities.
However, regular physical activity is vital for reducing the risk of diabetes and promoting overall health.
Physical activity helps improve insulin sensitivity, aids in weight management, and reduces the risk of various chronic conditions, including diabetes. We want to support you in finding ways to incorporate more physical activity into your daily routine. Here are some suggestions to consider:
Start Small: If you are not used to regular exercise, begin with short sessions of physical activity and gradually increase the duration and intensity as you feel more comfortable.
Find Activities You Enjoy: Engage in physical activities that you find enjoyable, such as walking, dancing, swimming, or cycling. Enjoying your workouts increases the likelihood of sticking to them.
Make It a Habit: Set aside specific times each day or week for physical activity. Treating exercise as an essential part of your routine increases the chances of making it a consistent habit.
Involve Friends or Family: Consider exercising with friends or family members. It can make the experience more enjoyable and provide additional motivation and accountability.
Break It Up: If finding 30 minutes at once is challenging, try breaking your physical activity into smaller sessions throughout the day. Even short bursts of activity can be beneficial.
Use Technology: Consider using fitness trackers or smartphone apps to monitor your activity levels and set goals. They can be valuable tools for staying motivated.
Consult a Professional: If you have any health concerns or limitations, consult with a healthcare provider or a certified fitness professional before starting a new exercise routine.
We understand that making lifestyle changes can be daunting, but even small steps towards a more active lifestyle can yield significant benefits for your health. Your well-being is our priority, and we are here to support you throughout your journey.
If you have any questions or need further guidance on incorporating physical activity into your life, please do not hesitate to reach out. Together, we can work towards better health and diabetes prevention.

### An Exemplary Follow-Up to Point 4 if Problematic

Subject: Follow-up on Diabetes Risk Assessment Survey-Diet
Dear [Patient's Name],
Thank you for participating in our Diabetes Risk Assessment Survey and sharing your insights on your dietary habits. Your commitment to understanding your health and well-being is commendable.
Based on your responses, it appears that there may be room for improvement in your diet to reduce the risk of diabetes and promote overall well-being. A healthy diet is a crucial factor in preventing and managing diabetes, and we want to support you in making positive changes to your eating habits.
Here are some practical tips to help you adopt a healthier diet:
Balanced Meals: Aim to have balanced meals that include a variety of food groups, such as fruits, vegetables, whole grains, lean proteins, and healthy fats.
Limit Sugary Foods and Beverages: Reduce the consumption of sugary foods and beverages, including soda, candies, cookies, and sweetened snacks.
Choose Whole Grains: Opt for whole grains such as brown rice, quinoa, and whole wheat bread instead of refined grains.
Portion Control: Pay attention to portion sizes to avoid overeating and unnecessary calorie intake.
Hydration: Stay hydrated by drinking plenty of water throughout the day. Limit the intake of sugary drinks and fruit juices.
Cook at Home: Preparing meals at home gives you better control over the ingredients and allows you to choose healthier options.
Plan Ahead: Plan your meals and snacks in advance to avoid impulsive and unhealthy choices.
Include Fruits and Vegetables: Aim to include a variety of colorful fruits and vegetables in your daily meals.
Healthy Snacking: Choose healthier snacks like nuts, fruits, yogurt, or raw vegetables instead of processed snacks.
Mindful Eating: Practice mindful eating by eating slowly, savoring the flavors, and paying attention to hunger and fullness cues.
Remember that making dietary changes takes time and effort, and it is essential to approach it with a positive and patient mindset. Small, sustainable changes can lead to significant improvements in your health.
If you find it challenging to modify your diet on your own, consider seeking guidance from a registered dietitian or nutritionist. They can provide personalized advice and create a plan that suits your individual needs and preferences.
We are here to support you throughout your health journey. If you have any questions or need further assistance in adopting a healthier diet, please don't hesitate to reach out to us.
Your well-being is of utmost importance, and together, we can work towards reducing your risk of diabetes and enhancing your overall health.

### An Exemplary Follow-Up to Point 5 if Problematic

Subject: Follow-up on Diabetes Risk Assessment Survey-Age
Dear [Patient's Name],
Thank you for participating in our Diabetes Risk Assessment Survey and providing us with important information about your health. Your dedication to understanding your risk factors is commendable.
We noticed that you indicated your current age in the survey. It is essential to recognize that age is a significant factor in diabetes risk, as the likelihood of developing diabetes increases with age. However, age is just one piece of the puzzle, and there are various ways to manage and reduce diabetes risk at any stage of life.
While we cannot control our age, we can take proactive steps to promote overall health and minimize the risk of diabetes. Here are some suggestions to consider:
Regular Health Check-ups: As you age, regular health check-ups become even more important. Schedule routine visits with your healthcare provider to monitor your blood glucose levels, blood pressure, cholesterol, and other key health indicators.
Healthy Eating Habits: A balanced and nutritious diet plays a crucial role in diabetes prevention. Focus on incorporating a variety of fruits, vegetables, whole grains, lean proteins, and healthy fats into your meals.
Physical Activity: Engaging in regular physical activity can help maintain a healthy weight, improve insulin sensitivity, and enhance overall well-being. Choose activities you enjoy and make exercise a part of your routine.
Maintain a Healthy Weight: If you are overweight, even modest weight loss can significantly reduce diabetes risk. Aim for gradual, sustainable weight loss through a combination of a healthy diet and regular exercise.
Manage Blood Pressure and Cholesterol: High blood pressure and cholesterol levels can increase the risk of diabetes complications. Work with your healthcare provider to monitor and manage these factors effectively.
Regular Screenings: As you age, you may be at increased risk for other health conditions that can impact diabetes risk, such as heart disease and kidney problems. Regular screenings can help detect and manage these conditions early.
Stress Management: Chronic stress can affect blood glucose levels and overall health.
Practice stress-reducing techniques such as mindfulness, meditation, or engaging in hobbies you enjoy.
Stay Informed: Stay up-to-date with the latest information about diabetes prevention, management, and healthy aging. Knowledge empowers you to make informed decisions about your health.
Remember, age is just one aspect of your health profile, and taking positive steps towards a healthy lifestyle can have a significant impact on your overall well-being. We are here to support you in managing your health and reducing your risk of diabetes at every stage of life.
If you have any questions or need further guidance, please do not hesitate to reach out to us.
Your well-being is our top priority, and we are committed to helping you achieve and maintain optimal health.

### An Exemplary Follow-Up to Point 6 if Problematic

Subject: Follow-up on Diabetes Risk Assessment Survey-Ethnicity
Dear [Patient's Name],
Thank you for participating in our Diabetes Risk Assessment Survey and sharing your responses with us. Your commitment to understanding your health and potential risk factors is commendable.
We noticed that you indicated your ethnicity in the survey. It's essential to recognize that certain ethnic backgrounds can be associated with a higher predisposition to developing diabetes. However, it is crucial to understand that diabetes risk is influenced by a combination of factors, including genetics, lifestyle, and environmental influences.
If you belong to an ethnic group with a higher prevalence of diabetes, it becomes even more critical to focus on proactive health measures to reduce your risk. Here are some steps you can consider taking to promote optimal health and diabetes prevention:
Healthy Eating Habits: Adopt a balanced and nutritious diet that is rich in fruits, vegetables, whole grains, and lean proteins. Limit the consumption of sugary and processed foods.
Physical Activity: Engage in regular physical activity that you enjoy, as exercise can improve insulin sensitivity and overall well-being.
Regular Health Check-ups: Schedule regular check-ups with your healthcare provider to monitor your blood glucose levels, blood pressure, cholesterol, and other vital health markers.
Weight Management: If you are overweight, consider working towards achieving a healthy weight through a combination of a balanced diet and regular exercise.
Know Your Family History: Be aware of your family's medical history, especially regarding diabetes. Knowing your family's health background can help you and your healthcare provider assess your risk more accurately.
Cultural Considerations: Embrace cultural practices that promote health and well-being while being mindful of aspects that may contribute to diabetes risk. Discuss any concerns with your healthcare provider to find suitable solutions.
Education and Support: Stay informed about diabetes prevention and management strategies.
Consider joining support groups or seeking educational resources to stay motivated and engaged in your health journey.
Remember, diabetes risk is not solely determined by ethnicity, and adopting a healthy lifestyle can significantly reduce your risk regardless of your background. We are here to support you in managing your health and achieving your wellness goals.
If you have any questions or need further guidance, please don't hesitate to reach out to us.
Your well-being is our top priority, and we are dedicated to helping you lead a healthy and fulfilling life.

### An Exemplary Follow-Up to Point 7 if Problematic

Subject: Follow-up on Diabetes Risk Assessment Survey-Gestational Diabetes
Dear [Patient's Name],
Thank you for participating in our Diabetes Risk Assessment Survey and providing us with valuable information about your health history. Your commitment to understanding your health and potential risk factors is commendable.
We noticed that you mentioned having been diagnosed with gestational diabetes during a previous pregnancy. Gestational diabetes is a type of diabetes that develops during pregnancy and can increase the risk of developing type 2 diabetes later in life.
As a healthcare provider, it is crucial for us to address this concern and provide you with the support and guidance you need to manage your health effectively. Here are some key points to consider:
Postpartum Testing: If you have experienced gestational diabetes, it is essential to undergo postpartum testing to assess your blood glucose levels and determine if you have returned to normal levels. Your healthcare provider can recommend the appropriate testing schedule.
Monitoring and Management: Even if your blood glucose levels return to normal after pregnancy, it is important to continue monitoring your health regularly. Adopting a healthy lifestyle that includes a balanced diet, regular physical activity, and stress management can help reduce the risk of type 2 diabetes.
Future Pregnancies: If you are planning future pregnancies, it is crucial to discuss your previous gestational diabetes diagnosis with your healthcare provider. They can provide guidance on managing diabetes risk during pregnancy.
Preconception Planning: For women planning to conceive, preconception planning is essential. Working closely with your healthcare provider before becoming pregnant can help optimize your health and reduce the risk of gestational diabetes recurrence.
Education and Support: Stay informed about gestational diabetes and its potential implications on your long-term health. Seek educational resources and support from healthcare professionals to make informed decisions about your well-being.
Remember that gestational diabetes does not mean you will definitely develop type 2 diabetes, but it does increase your risk. By being proactive about your health and making positive lifestyle changes, you can significantly reduce that risk and promote overall well-being.
If you have any questions or concerns about managing gestational diabetes or reducing your diabetes risk, please don't hesitate to reach out to us. We are here to support you on your health journey.
Your well-being is our priority, and together, we can work towards a healthier and happier future.

### An Exemplary Follow-Up to Point 8 if Problematic

Subject: Follow-up on Diabetes Risk Assessment Survey-Polycystic Ovary Syndrome (PCOS)
Dear [Patient's Name],
Thank you for participating in our Diabetes Risk Assessment Survey and providing us with important information about your health. Your dedication to understanding your risk factors is commendable.
We noticed that you mentioned being diagnosed with Polycystic Ovary Syndrome (PCOS).
PCOS is a hormonal disorder that can lead to insulin resistance and an increased risk of type 2 diabetes. It's essential to address this concern and provide you with the support and guidance you need to manage your health effectively.
Managing PCOS and reducing the risk of diabetes often involves a comprehensive approach that includes lifestyle modifications and, in some cases, medical interventions. Here are some key points to consider:
Lifestyle Modifications: Adopting a healthy lifestyle can significantly improve the management of PCOS and reduce the risk of diabetes. Focus on a balanced diet, regular physical activity, and stress management.
Healthy Eating Habits: Emphasize foods with a low glycemic index, such as whole grains, fruits, vegetables, and lean proteins. Limit the intake of sugary and processed foods.
Physical Activity: Engage in regular physical activity to improve insulin sensitivity and support overall health. Aim for at least 150 minutes of moderate-intensity exercise per week.
Medication: In some cases, medication may be prescribed to manage PCOS symptoms and insulin resistance. Follow your healthcare provider's recommendations and take medications as prescribed.
Regular Check-ups: Schedule regular check-ups with your healthcare provider to monitor your PCOS symptoms, blood glucose levels, and overall health.
Weight Management: If you are overweight, achieving and maintaining a healthy weight can be beneficial in managing PCOS and reducing diabetes risk.
Fertility and Pregnancy Planning: If you are planning to conceive, discuss your PCOS diagnosis with your healthcare provider. They can provide guidance on managing PCOS-related fertility concerns and diabetes risk during pregnancy.
Education and Support: Stay informed about PCOS, diabetes, and their potential implications on your health. Seek educational resources and support from healthcare professionals to make informed decisions about your well-being.
It's essential to remember that managing PCOS and reducing diabetes risk is a journey that requires dedication and patience. By working closely with your healthcare provider and making positive lifestyle changes, you can improve your overall health and well-being.
If you have any questions or concerns about managing PCOS or reducing your diabetes risk, please don't hesitate to reach out to us. We are here to support you on your health journey.
Your well-being is our priority, and together, we can work towards a healthier and happier future.

### An Exemplary Follow-Up to Point 9 if Problematic

Subject: Follow-up on Diabetes Risk Assessment Survey—High Blood Pressure (Hypertension)

Dear [Patient's Name],

Thank you for participating in our Diabetes Risk Assessment Survey and sharing important information about your health. Your commitment to understanding your risk factors is commendable.

We noticed that you mentioned being diagnosed with high blood pressure (hypertension).

Hypertension is a significant risk factor for diabetes, as it can contribute to insulin resistance and increase the risk of developing type 2 diabetes.

Managing hypertension is crucial for your overall health, and it can also play a pivotal role in reducing diabetes risk. Here are some key points to consider:

Blood Pressure Monitoring: Regularly monitor your blood pressure as advised by your healthcare provider. Keeping track of your readings can help you and your healthcare team assess your progress.

Medication Compliance: If you have been prescribed medication to manage hypertension, it's essential to take the medication as prescribed by your healthcare provider. Consistent medication compliance is vital for effective blood pressure control.

Lifestyle Modifications: Lifestyle changes, including a healthy diet and regular physical activity, can complement medical treatments and contribute to better blood pressure management.

Healthy Eating Habits: Adopt a balanced diet that includes plenty of fruits, vegetables, whole grains, and lean proteins. Limit sodium intake and reduce the consumption of processed and high-sodium foods.

Physical Activity: Engage in regular physical activity to help lower blood pressure and improve overall cardiovascular health. Aim for at least 150 minutes of moderate-intensity exercise per week.

Weight Management: If you are overweight, losing even a small amount of weight can have a positive impact on blood pressure and diabetes risk.

Stress Management: Chronic stress can affect blood pressure levels. Explore stress-reducing techniques, such as mindfulness, meditation, or engaging in hobbies you enjoy.

Limit Alcohol and Tobacco Use: Reducing alcohol consumption and quitting smoking, if applicable, can significantly contribute to blood pressure control and overall health.

Regular Check-ups: Regularly visit your healthcare provider to monitor your blood pressure, diabetes risk, and overall health.

Managing hypertension requires a comprehensive approach that combines medical interventions and lifestyle changes. By taking proactive steps towards better health, you can reduce your risk of diabetes and promote overall well-being.

If you have any questions or concerns about managing hypertension or reducing diabetes risk, please don't hesitate to reach out to us. We are here to support you on your health journey.

Your well-being is our priority, and together, we can work towards a healthier and happier future.

### An Exemplary Follow-Up to Point 10 if Problematic

Subject: Follow-up on Diabetes Risk Assessment Survey-Chronic Inflammation

Dear [Patient's Name],

Thank you for participating in our Diabetes Risk Assessment Survey and providing us with valuable information about your health. Your dedication to understanding your risk factors is commendable.

We noticed that you mentioned having chronic inflammation in the survey. Chronic inflammation is a complex condition that can contribute to insulin resistance and increase the risk of developing type 2 diabetes.

Managing chronic inflammation is essential for your overall health, and it can also play a significant role in reducing diabetes risk. While addressing chronic inflammation may require a comprehensive approach, there are steps you can take to promote better health. Here are some key points to consider: Consult with a Healthcare Provider: If you have been diagnosed with chronic inflammation or suspect its presence, it's crucial to consult with your healthcare provider. They can help assess the extent of inflammation and provide appropriate guidance.

Identify Underlying Causes: Work with your healthcare provider to identify and address any underlying causes of chronic inflammation, such as infections, autoimmune conditions, or lifestyle factors.

Healthy Eating Habits: Adopt a balanced diet that includes anti-inflammatory foods, such as fatty fish (rich in omega-3 fatty acids), fruits, vegetables, nuts, and seeds.

Physical Activity: Engage in regular physical activity, as exercise can help reduce inflammation and improve overall well-being.

Weight Management: If you are overweight, achieving a healthy weight can help reduce inflammation and improve insulin sensitivity.

Stress Management: Chronic stress can contribute to inflammation. Explore stress-reducing techniques, such as mindfulness, meditation, or yoga.

Avoid Smoking and Limit Alcohol: Smoking and excessive alcohol consumption can contribute to inflammation and have negative effects on your health.

Medication and Treatment: If prescribed by your healthcare provider, take any medications or follow treatments aimed at managing chronic inflammation.

Regular Check-ups: Regularly visit your healthcare provider to monitor your health and address any concerns related to chronic inflammation or diabetes risk.

Managing chronic inflammation requires a personalized approach, and working closely with your healthcare provider is essential to tailor a plan that suits your individual needs.

If you have any questions or concerns about managing chronic inflammation or reducing diabetes risk, please don't hesitate to reach out to us. We are here to support you on your health journey.

Your well-being is our priority, and together, we can work towards a healthier and happier future.

### An Exemplary Follow-Up to Point 11 if Problematic

Subject: Follow-up on Diabetes Risk Assessment Survey—Smoking

Dear [Patient's Name],

Thank you for participating in our Diabetes Risk Assessment Survey and sharing valuable information about your health. Your commitment to understanding your risk factors is commendable.

We noticed that you mentioned smoking or using tobacco products in the survey. Smoking is a significant risk factor for numerous health conditions, including type 2 diabetes. It can contribute to insulin resistance, increase blood sugar levels, and exacerbate diabetes-related complications.

As your healthcare provider, it is our responsibility to address this concern and provide you with the support and resources you need to make positive changes to your health. Here are some key points to consider:

Quitting Smoking: If you are currently smoking, we strongly encourage you to quit. Quitting smoking is one of the most impactful steps you can take to improve your overall health and reduce diabetes risk.

Smoking Cessation Resources: There are various resources available to help you quit smoking, including smoking cessation programs, support groups, and medications. Discuss your options with your healthcare provider to find the best approach for you.

Lifestyle Modifications: Adopt a healthy lifestyle that includes a balanced diet, regular physical activity, and stress management. These positive changes can complement your efforts to quit smoking and improve your overall well-being.

Identify Triggers: Recognize triggers that may lead to smoking and develop strategies to cope with them. Finding healthier alternatives to manage stress or boredom can be beneficial during the quitting process.

Support System: Reach out to family, friends, or support groups for encouragement and accountability during your smoking cessation journey.

Health Benefits: Quitting smoking has immediate and long-term health benefits, including reduced diabetes risk, improved lung function, and decreased risk of cardiovascular diseases.

Regular Check-ups: Continue to schedule regular check-ups with your healthcare provider to monitor your health and receive ongoing support.

Remember, quitting smoking is a challenging process, but you don't have to do it alone. We are here to support you every step of the way. Your well-being is our priority, and we are dedicated to helping you achieve better health and reduce your risk of diabetes.

If you have any questions or need further guidance on smoking cessation or diabetes risk reduction, please don't hesitate to reach out to us. Together, we can work towards a healthier and happier future.

### An Exemplary Follow-Up to Point 12 if Problematic

Subject: Follow-up on Diabetes Risk Assessment Survey-Stress Management

Dear [Patient's Name],

Thank you for participating in our Diabetes Risk Assessment Survey and providing us with important information about your health. Your commitment to understanding your risk factors is commendable.

We noticed that you mentioned your overall stress level in the survey. Chronic stress can have a significant impact on your health, including affecting blood glucose levels and overall well-being.

Stress is associated with insulin resistance and an increased risk of developing type 2 diabetes.

Managing stress is crucial for your overall health and can also play a significant role in reducing diabetes risk. While eliminating stress entirely may not be possible, there are strategies you can adopt to better manage stress and promote well-being. Here are some key points to consider:

Identify Stressors: Recognize the factors or situations that contribute to your stress. Understanding your stressors can help you develop targeted strategies for managing them.

Stress-Reduction Techniques: Explore stress-reduction techniques such as mindfulness, meditation, deep breathing exercises, yoga, or engaging in hobbies you enjoy.

Physical Activity: Regular physical activity can be an effective way to reduce stress and improve mood. Choose activities you enjoy and incorporate them into your routine.

Healthy Eating Habits: A balanced diet can support your body's ability to cope with stress. Include nutrient-rich foods and avoid excessive consumption of sugary and processed foods.

Time Management: Organize your schedule and prioritize tasks to reduce feelings of overwhelm.

Proper time management can help create a sense of control over your day.

Social Support: Stay connected with friends and family and seek support when needed. Sharing your feelings and concerns can help reduce stress.

Limit Exposure to Stress: Minimize exposure to stressful situations when possible. Setting healthy boundaries and learning to say “no” can be empowering.

Quality Sleep: Prioritize getting enough restful sleep, as lack of sleep can exacerbate stress and impact your overall health.

Professional Help: If stress becomes overwhelming or affects your daily life, consider seeking support from a mental health professional or counselor.

Regular Check-ups: Schedule regular check-ups with your healthcare provider to monitor your health and discuss any concerns related to stress and diabetes risk.

Remember that managing stress is a journey that requires patience and practice. By adopting positive coping strategies, you can enhance your well-being and reduce your risk of diabetes.

If you have any questions or concerns about stress management or reducing diabetes risk, please don't hesitate to reach out to us. We are here to support you on your health journey.

Your well-being is our priority, and together, we can work towards a healthier and happier future.

### Personalized Work Plan

To help the patient make progress over time on point 1 (Genetic Factors—Family History of Diabetes), a personalized plan is developed. This plan should focus on educating the patient about diabetes risk associated with family history and implementing lifestyle changes to reduce the impact of genetic factors. One step-by-step plan includes:

Patient Education: Explain the significance of family history in diabetes risk; Provide information on the genetic factors that can contribute to the development of diabetes; Discuss how lifestyle choices can modify the influence of genetic factors.

Health Check-ups: Schedule regular health check-ups to monitor blood glucose levels and overall health; Set specific intervals for follow-up visits based on the patient's risk profile and individual needs.

Family History Analysis: Encourage the patient to gather detailed information about their family's medical history, particularly related to diabetes; Identify other potential risk factors in the family history that may contribute to diabetes risk.

Personalized Diet Plan: Collaborate with a registered dietitian to create a balanced and nutritious diet plan tailored to the patient's preferences and lifestyle; Emphasize the importance of whole foods, portion control, and reducing the intake of sugary and processed foods.

Physical Activity Recommendations: Work with the patient to develop a personalized exercise routine based on their fitness level, preferences, and any existing health conditions; Encourage regular physical activity, aiming for at least 150 minutes of moderate-intensity exercise per week.

Stress Management: Discuss stress management techniques such as mindfulness, meditation, or relaxation exercises; Offer resources or referrals to support groups or counseling if needed.

Support System: Encourage the patient to involve their immediate family members in their health journey; Seek support from family and friends in adopting and maintaining healthy lifestyle changes.

Monitoring Progress: Set achievable short-term and long-term goals related to diet, exercise, and overall health; Regularly assess the patient's progress in making lifestyle changes and adjust the plan accordingly.

Education Materials: Provide educational materials, pamphlets, or online resources on diabetes risk factors and lifestyle modifications; Offer resources that explain how genetic factors interact with lifestyle choices.

Motivation and Encouragement: Offer positive reinforcement and encouragement throughout the patient's health journey; Celebrate small achievements and milestones to boost motivation.

Regular Follow-ups: Schedule follow-up appointments at appropriate intervals to track progress and provide ongoing support; Use these appointments to address any challenges or concerns the patient may be facing.

Collaborative Approach: Involve a multidisciplinary healthcare team, including dietitians, nurses, and psychologists, to provide comprehensive support to the patient.

The system continuously tailors the plan based on the patient's responses, preferences, and needs. By implementing this plan and offering ongoing support, the patient can make positive strides in reducing their diabetes risk, even in the presence of genetic factors.

Gamification is an effective strategy to engage and motivate patients in making progress on their health goals, including point 2 (Obesity and Weight Management). By incorporating elements of gamification into the patient's weight management plan, it can become more enjoyable and rewarding.

### Here's a Gamification Plan to Help the Patient Make Progress Over Time:

Health Quests: Create “health quests” or challenges related to weight management. These quests could include achieving specific weight loss milestones, completing a certain number of workouts, or making healthier food choices for a designated period. Each completed quest can earn the patient points or rewards.

Personalized Avatar: Design a virtual avatar for the patient that represents their progress. As the patient achieves weight loss milestones or completes challenges, the avatar's appearance can change to reflect their achievements.

Progress Tracking: Use a mobile app or digital platform to track the patient's weight loss progress, physical activity, and dietary habits. Visualizing progress through charts and graphs can be motivating.

Healthy Habit Streaks: Encourage the patient to build streaks of consistent healthy habits. For example, they can earn points for exercising for five consecutive days or choosing healthy meals throughout the week.

Social Support: Integrate a social aspect into the gamification plan. Allow the patient to connect with other individuals with similar health goals, share achievements, and provide support to one another.

Rewards System: Establish a reward system where the patient can redeem their earned points for tangible or intangible rewards. Rewards can include gift cards, fitness equipment, or even recognition and praise from the healthcare team.

Leveling Up: Set different levels of achievement for weight loss milestones. As the patient reaches a new level, they unlock new challenges and rewards, encouraging them to strive for higher achievements.

Friendly Competitions: Organize friendly competitions or challenges between patients to boost motivation. These could include step challenges, healthy recipe cook-offs, or team-based fitness activities.

Educational Quizzes: Integrate educational quizzes or mini-games related to nutrition, exercise, and weight management. Patients can earn points for answering questions correctly, promoting continuous learning.

Incorporate Real-world Rewards: Partner with local businesses to offer discounts or special deals on healthy products or services as rewards for patient achievements.

Virtual Badges and Trophies: Award virtual badges or trophies to the patient for reaching significant milestones or consistently following their weight management plan.

Regular Check-ins: Schedule regular check-ins with the patient to discuss their progress, offer encouragement, and adjust the gamification plan as needed based on their feedback and experiences.

By implementing this gamification plan, the patient can find weight management more enjoyable, engaging, and rewarding. The element of fun and challenge can help boost motivation and adherence to their weight loss goals over time.

Creating a gamification plan to help the patient make progress over time on point 3 (Unhealthy Eating Habits) can encourage them to adopt healthier dietary choices. Gamification can make the process enjoyable and motivate the patient to sustain positive changes. Here's a gamification plan:

Healthy Eating Challenges: Develop weekly or monthly healthy eating challenges. These challenges could include increasing fruit and vegetable intake, reducing added sugars, or trying new healthy recipes.

Nutrition Quests: Create quests that focus on specific nutritional goals, such as consuming a certain amount of fiber, drinking enough water, or balancing macronutrients.

Food Diary: Encourage the patient to keep a food diary to track their meals and snacks. Use a mobile app or online tool for easy logging.

Meal Planning Rewards: Reward the patient for planning their meals in advance. Provide extra points or bonuses for creating balanced and nutritious meal plans.

Healthy Recipe Exchange: Set up a platform for patients to share their favorite healthy recipes. They can earn points for sharing recipes and trying out recipes shared by others.

Educational Quizzes: Host quizzes or trivia games related to nutrition and healthy eating. Patients can earn points for participating and answering questions correctly.

Virtual Grocery Shopping: Create a virtual grocery store where patients can “shop” for healthy items using their earned points. This can reinforce the value of making nutritious choices.

Portion Control Challenge: Encourage portion control by setting up a challenge to help patients learn proper serving sizes. They can earn rewards for practicing portion control consistently.

Mealtime Mindfulness: Promote mindful eating habits by encouraging patients to savor each bite and eat without distractions. Points can be awarded for practicing mindful eating.

Nutrient Tracking: Gamify nutrient tracking by rewarding patients for meeting daily recommended intake levels of essential nutrients like vitamins and minerals.

Healthy Eating Streaks: Encourage the patient to build streaks of consecutive days where they meet their healthy eating goals. Longer streaks can earn more significant rewards.

Social Support: Establish a community or group where patients can share their healthy eating journey, exchange tips, and provide support to one another.

Virtual Chef: Create a virtual cooking challenge where patients can submit pictures of their healthy meals. The most creative and nutritious entries can win rewards.

Cooking Classes: Offer virtual cooking classes focused on preparing healthy meals. Patients can earn points for attending and applying what they learn.

Progress Badges: Award virtual badges or stars for reaching milestones, such as completing a specific number of healthy eating challenges or logging meals consistently.

Regular Check-ins: Schedule regular check-ins with the patient to review their dietary progress, provide feedback, and adjust the gamification plan based on their needs.

By implementing this gamification plan, the patient can be motivated to adopt healthier eating habits gradually. The element of fun and competition can encourage them to make lasting improvements in their diet over time.

Creating a gamification plan to help the patient make progress over time on point 4 (Sedentary Lifestyle) can encourage them to become more physically active and lead a healthier lifestyle.

Gamification can make the process enjoyable, motivating the patient to sustain positive changes.

### Here's a Gamification Plan:

Daily Step Challenge: Set a daily step goal for the patient and track their steps using a fitness tracker or smartphone app. Reward them with points or badges for meeting or exceeding their daily step target.

Activity Points: Implement a points system where the patient can earn points for engaging in various physical activities, such as walking, jogging, cycling, or dancing.

Virtual Adventure: Create a virtual adventure where the patient can virtually explore different locations based on the number of steps they take. Unlock new destinations and landmarks as they accumulate more steps.

Team Competitions: Organize friendly team competitions among patients to encourage camaraderie and friendly rivalry. Teams can earn points collectively based on their physical activity levels.

Personalized Avatars: Design virtual avatars that represent the patient's physical activity progress. As they reach specific milestones, their avatars can level up or unlock new features.

Fitness Challenges: Introduce fitness challenges, such as a 30-day plank challenge or a 7-day push-up challenge. Patients can earn points for completing these challenges successfully.

Interval Training Quests: Encourage patients to incorporate interval training into their routines. They can earn points for completing a certain number of interval workouts per week.

Fitness Classes: Offer virtual fitness classes that patients can attend and earn points for participating.

Classes can include yoga, Zumba, HIIT, and more.

Badges and Rewards: Award virtual badges or rewards for meeting specific milestones, such as walking a certain distance, completing a certain number of workouts, or achieving a target heart rate.

Health Streaks: Reward patients for maintaining consistent physical activity streaks, such as exercising for a certain number of consecutive days.

Activity Challenges: Host activity challenges, where patients compete to complete a certain number of minutes of physical activity within a specified period. Winners can receive recognition and additional points.

Real-world Rewards: Partner with local fitness centers, sports stores, or wellness companies to offer real-world rewards for patients who achieve significant milestones.

Progress Tracking: Use a fitness app or platform to track the patient's physical activity progress, displaying achievements, and providing insights into their journey.

Social Interaction: Create a community or group where patients can share their activity progress, exchange fitness tips, and provide encouragement to one another.

Regular Check-ins: Schedule regular check-ins with the patient to review their physical activity progress, discuss challenges, and adjust the gamification plan based on their needs.

By implementing this gamification plan, the patient can be motivated to adopt a more active lifestyle gradually. The element of fun, competition, and rewards can encourage them to sustain positive changes and lead a healthier, more active life over time.

Creating a gamification plan to help the patient make progress over time on point 5 (Age) can be instrumental in encouraging them to prioritize their health and address age-related risk factors.

Gamification can make the process enjoyable and motivating, fostering healthy habits. Here's a gamification plan:

Health Quests by Age Group: Tailor health quests and challenges based on the patient's specific age group. For example, different challenges can be designed for patients in their 40s, 50s, 60s, etc., addressing age-appropriate health concerns.

Virtual Age Milestones: Create virtual age milestones as the patient celebrates each birthday. Upon reaching a new age group, they can unlock new challenges and receive congratulatory rewards.

Age-Appropriate Exercise Goals: Set exercise goals that align with the patient's age and fitness level.

As they meet these goals, they earn points and progress to more advanced challenges.

Age Group Leaderboards: Implement age-specific leaderboards, so patients can compare their progress with others in their age group. This fosters a sense of friendly competition and camaraderie.

Education on Age-Related Risks: Offer educational quizzes or modules on age-related health risks and preventive measures. Patients can earn points for completing educational activities.

Functional Fitness Challenges: Design functional fitness challenges that cater to the patient's age and focus on activities that improve their daily living skills and overall well-being.

Memory and Brain Health Games: Incorporate brain-boosting games and puzzles to promote cognitive health. Patients can earn rewards for actively engaging in brain-stimulating activities.

Joint Health Tracking: Encourage the patient to track joint health and flexibility progress through a mobile app or wearable device. They can earn points for taking care of their joints.

Longevity Streaks: Reward patients for maintaining consistency in their health routines by establishing “longevity streaks.” For example, they can earn bonus points for exercising continuously for a certain number of days or weeks.

Age-Defying Recipes: Share age-defying recipes that focus on nutrients beneficial for their age group.

Patients can earn points for trying out and sharing healthy recipes.

Healthy Aging Stories: Create a platform where patients can share their stories of healthy aging and inspire others to make positive changes. The most inspiring stories can receive recognition and additional rewards.

Positive Aging Affirmations: Provide daily or weekly positive affirmations related to healthy aging.

Patients can earn points for reflecting on and applying these affirmations in their lives.

Social Support: Establish a community where patients can connect with others in their age group, share experiences, and provide encouragement on their health journey.

Regular Check-ins: Schedule regular check-ins with the patient to review their progress on age-related health goals, offer guidance, and adjust the gamification plan based on their feedback and needs.

By implementing this gamification plan, the patient can be motivated to embrace healthy aging practices and prioritize their well-being. The gamified approach can make their health journey engaging and rewarding, leading to sustained positive changes over time.

Creating a gamification plan to help the patient make progress over time on point 6 (Medical Conditions—Prediabetes, Metabolic Syndrome) can encourage them to take proactive steps in managing their health and reducing the risk of developing type 2 diabetes. Gamification can make the process enjoyable and motivating, fostering adherence to healthy lifestyle changes. Here's a gamification plan:

Prediabetes Quest: Design a “Prediabetes Quest” where the patient sets specific health goals related to blood glucose levels, weight management, and physical activity. They can earn points for achieving these goals.

Metabolic Syndrome Tracker: Implement a tracker to monitor key indicators of metabolic syndrome, such as blood pressure, waist circumference, and cholesterol levels. Patients can earn rewards for improving these metrics.

Blood Glucose Challenge: Create challenges related to blood glucose management. Patients can earn points for maintaining stable blood sugar levels and reducing HbA1c within a specific target range.

Physical Activity Challenges: Develop physical activity challenges tailored to the patient's fitness level and medical condition. They can earn points for participating in activities like walking, cycling, or strength training.

Nutrition Targets: Set personalized nutrition targets to address the patient's medical conditions. They can earn points for meeting daily goals, such as consuming a certain amount of fiber or limiting added sugars.

Medication Adherence Rewards: If the patient is prescribed medication, reward them for adhering to their medication schedule and monitoring their response to treatment.

Health Insights: Use a mobile app or platform to provide health insights and recommendations based on the patient's data. Patients can earn points for reviewing and acting on these insights.

Virtual Coaching: Offer virtual coaching sessions with healthcare professionals to provide personalized guidance and support. Patients can earn points for attending coaching sessions.

Medical Team Rewards: Encourage collaboration with the patient's medical team. Reward patients for scheduling regular check-ups, following their healthcare provider's advice, and achieving health targets.

Metabolic Makeover: Create a “Metabolic Makeover” challenge where the patient commits to making positive lifestyle changes for a specified period. They can earn bonus points for completing the makeover successfully.

Personalized Educational Modules: Provide educational modules tailored to the patient's medical conditions. Patients can earn points for completing these modules and demonstrating their knowledge.

Social Support Network: Establish a supportive community where patients can connect with others facing similar challenges. They can share experiences, offer encouragement, and receive motivation.

Stress Reduction Quest: Offer stress-reduction challenges like mindfulness exercises or relaxation techniques. Patients can earn points for participating in stress-reducing activities.

Health Journey Journal: Encourage patients to maintain a health journey journal to track their progress, setbacks, and reflections. They can earn points for regularly updating their journal.

Regular Check-ins: Schedule regular check-ins with the patient to review their health progress, celebrate achievements, and adjust the gamification plan based on their feedback and needs.

By implementing this gamification plan, the patient can be motivated to take an active role in managing their medical conditions and reducing their risk of developing type 2 diabetes. The gamified approach can make their health journey engaging and rewarding, leading to sustained positive changes over time.

Creating a gamification plan to help the patient make progress over time on point 7 (Polycystic Ovary Syndrome—PCOS) can encourage them to manage their condition effectively and reduce the risk of complications. Gamification can make the process enjoyable and motivating, fostering adherence to PCOS management strategies. Here's a gamification plan:

PCOS Power Challenges: Develop “PCOS Power Challenges” where the patient sets health goals related to managing PCOS symptoms, such as improving insulin sensitivity or promoting hormonal balance. They can earn points for achieving these goals.

Hormonal Harmony Tracker: Implement a tracker to monitor key hormonal indicators relevant to PCOS management, such as estrogen, progesterone, and androgen levels. Patients can earn rewards for maintaining hormonal balance.

Nutrition for PCOS: Set personalized nutrition targets that align with PCOS management. Patients can earn points for meeting dietary goals, such as consuming foods with a low glycemic index or incorporating PCOS-friendly recipes.

Physical Activity Boost: Create physical activity challenges tailored to the patient's fitness level and PCOS needs. They can earn points for participating in activities like strength training or exercises that improve insulin sensitivity.

Stress-Relief Quests: Offer stress-relief quests focused on managing stress, which can be beneficial for PCOS management. Patients can earn points for practicing mindfulness, yoga, or other relaxation techniques.

Medication and Treatment Adherence Rewards: If the patient is prescribed medication or treatments for PCOS, reward them for adhering to their treatment plan and following their healthcare provider's recommendations.

Hormonal Health Insights: Use a mobile app or platform to provide health insights specific to PCOS management. Patients can earn points for reviewing and acting on these insights.

Virtual Coaching Support: Offer virtual coaching sessions with healthcare professionals who specialize in PCOS management. Patients can earn points for attending coaching sessions.

PCOS Community: Establish a supportive community where patients with PCOS can connect with others facing similar challenges. They can share experiences, offer encouragement, and receive motivation.

Personalized Educational Modules: Provide educational modules tailored to PCOS management.

Patients can earn points for completing these modules and demonstrating their knowledge.

PCOS Progress Journal: Encourage patients to maintain a PCOS progress journal to track their symptoms, lifestyle changes, and reflections. They can earn points for regularly updating their journal.

Fertility and Pregnancy Planning: If the patient plans to conceive, offer quests and challenges related to PCOS management during fertility and pregnancy planning. They can earn bonus points for completing these challenges.

Regularity Reward: Encourage patients to track the regularity of their menstrual cycles. They can earn points for maintaining more regular cycles, which is a positive sign of PCOS management.

Regular Check-ins: Schedule regular check-ins with the patient to review their PCOS management progress, celebrate achievements, and adjust the gamification plan based on their feedback and needs.

By implementing this gamification plan, the patient can be motivated to actively manage their PCOS condition and reduce the risk of complications. The gamified approach can make their health journey engaging and rewarding, leading to sustained positive changes over time.

Creating a gamification plan to help the patient make progress over time on point 8 (High Blood Pressure-Hypertension) can encourage them to manage their blood pressure effectively and reduce the risk of cardiovascular complications. Gamification can make the process enjoyable and motivating, fostering adherence to hypertension management strategies. Here's a gamification plan: Blood Pressure Hero Challenges: Develop “Blood Pressure Hero Challenges” where the patient sets specific health goals related to managing blood pressure, such as achieving target blood pressure readings or reducing sodium intake. They can earn points for achieving these goals.

Hypertension Tracker: Implement a tracker to monitor blood pressure readings regularly. Patients can earn rewards for maintaining their blood pressure within target ranges.

Low-Sodium Quests: Set personalized nutrition targets that focus on reducing sodium intake. Patients can earn points for meeting daily sodium goals and discovering low-sodium recipes.

Physical Activity for Heart Health: Create physical activity challenges aimed at improving cardiovascular fitness. Patients can earn points for engaging in activities like brisk walking, swimming, or cycling.

Stress Reduction Activities: Offer stress-relief quests and challenges, as stress management is crucial for blood pressure control. Patients can earn points for practicing relaxation techniques, meditation, or spending time in nature.

Medication and Treatment Adherence Rewards: If the patient is prescribed medication or treatments for hypertension, reward them for adhering to their treatment plan and following their healthcare provider's recommendations.

Heart-Healthy Insights: Use a mobile app or platform to provide health insights related to hypertension management. Patients can earn points for reviewing and acting on these insights.

Virtual Coaching Support: Offer virtual coaching sessions with healthcare professionals specializing in hypertension management. Patients can earn points for attending coaching sessions.

Hydration Challenge: Encourage patients to maintain proper hydration as it can impact blood pressure. They can earn points for drinking an adequate amount of water daily.

Heart-Healthy Recipes: Share heart-healthy recipes that focus on nutrients beneficial for blood pressure management. Patients can earn points for trying out and sharing these recipes.

Community Support: Establish a supportive community where patients can connect with others managing hypertension. They can share experiences, offer encouragement, and receive motivation.

Personalized Educational Modules: Provide educational modules tailored to hypertension management. Patients can earn points for completing these modules and demonstrating their knowledge.

Blood Pressure Progress Journal: Encourage patients to maintain a blood pressure progress journal to track their readings, lifestyle changes, and reflections. They can earn points for regularly updating their journal.

Regular Check-ins: Schedule regular check-ins with the patient to review their blood pressure management progress, celebrate achievements, and adjust the gamification plan based on their feedback and needs.

By implementing this gamification plan, the patient can be motivated to actively manage their blood pressure and reduce the risk of cardiovascular complications. The gamified approach can make their health journey engaging and rewarding, leading to sustained positive changes over time.

Creating a gamification plan to help the patient make progress over time on point 9 (High Cholesterol—Hypercholesterolemia) can encourage them to manage their cholesterol levels effectively and reduce the risk of cardiovascular complications. Gamification can make the process enjoyable and motivating, fostering adherence to cholesterol management strategies. Here's a gamification plan:

Cholesterol Champion Challenges: Develop “Cholesterol Champion Challenges” where the patient sets specific health goals related to managing cholesterol levels, such as achieving target cholesterol readings or adopting a heart-healthy diet. They can earn points for achieving these goals.

Cholesterol Tracker: Implement a tracker to monitor cholesterol levels regularly. Patients can earn rewards for maintaining their cholesterol levels within target ranges.

Heart-Healthy Nutrition Quests: Set personalized nutrition targets that focus on heart-healthy foods, such as increasing fiber intake and reducing saturated fats. Patients can earn points for meeting daily dietary goals.

Physical Activity for Heart Health: Create physical activity challenges aimed at improving cardiovascular fitness and cholesterol management. Patients can earn points for engaging in activities like aerobic exercises, hiking, or dancing.

Stress Reduction Activities: Offer stress-relief quests and challenges, as stress management can impact cholesterol levels. Patients can earn points for practicing relaxation techniques, meditation, or yoga.

Medication and Treatment Adherence Rewards: If the patient is prescribed medication or treatments for high cholesterol, reward them for adhering to their treatment plan and following their healthcare provider's recommendations.

Heart-Healthy Insights: Use a mobile app or platform to provide health insights related to cholesterol management. Patients can earn points for reviewing and acting on these insights.

Virtual Coaching Support: Offer virtual coaching sessions with healthcare professionals specializing in cholesterol management. Patients can earn points for attending coaching sessions.

Healthy Fats Challenge: Encourage patients to focus on incorporating healthy fats into their diet, such as omega-3 fatty acids. They can earn points for consuming heart-healthy fats.

Cholesterol-Lowering Recipes: Share cholesterol-lowering recipes that focus on nutrients beneficial for managing cholesterol levels. Patients can earn points for trying out and sharing these recipes.

Community Support: Establish a supportive community where patients can connect with others managing high cholesterol. They can share experiences, offer encouragement, and receive motivation.

Personalized Educational Modules: Provide educational modules tailored to cholesterol management.

Patients can earn points for completing these modules and demonstrating their knowledge.

Cholesterol Progress Journal: Encourage patients to maintain a cholesterol progress journal to track their readings, lifestyle changes, and reflections. They can earn points for regularly updating their journal.

Regular Check-ins: Schedule regular check-ins with the patient to review their cholesterol management progress, celebrate achievements, and adjust the gamification plan based on their feedback and needs.

By implementing this gamification plan, the patient can be motivated to actively manage their cholesterol levels and reduce the risk of cardiovascular complications. The gamified approach can make their health journey engaging and rewarding, leading to sustained positive changes over time.

Creating a gamification plan to help the patient make progress over time on point 10 (Tobacco Use—Smoking Cessation) can encourage them to quit smoking and adopt a tobacco-free lifestyle.

Gamification can make the process enjoyable and motivating, fostering adherence to smoking cessation strategies. Here's a gamification plan:

Smoke-Free Challenge: Develop a “Smoke-Free Challenge” where the patient sets specific goals related to quitting smoking, such as reducing the number of cigarettes smoked per day or achieving smoke-free days. They can earn points for achieving these goals.

Tobacco-Free Tracker: Implement a tracker to monitor the patient's progress in quitting smoking.

Patients can earn rewards for meeting milestones, such as smoke-free weeks or months.

Nicotine Replacement Therapy (NRT) Support: Offer support and rewards for patients using NRT products, such as nicotine patches or gum, to aid in their smoking cessation journey.

Physical Activity to Combat Cravings: Create physical activity challenges aimed at distracting patients from tobacco cravings. They can earn points for engaging in activities like brisk walking, jogging, or dancing.

Stress Relief Activities: Offer stress-relief quests and challenges, as stress can be a trigger for smoking. Patients can earn points for practicing relaxation techniques, mindfulness, or meditation.

Medication and Treatment Adherence Rewards: If the patient is using smoking cessation medications, reward them for adhering to their treatment plan and following their healthcare provider's recommendations.

Benefits of Quitting Smoking Insights: Use a mobile app or platform to provide health insights related to the benefits of quitting smoking. Patients can earn points for reviewing and acknowledging these positive changes.

Virtual Coaching Support: Offer virtual coaching sessions with healthcare professionals specializing in smoking cessation. Patients can earn points for attending coaching sessions.

Tobacco-Free Milestone Celebrations: Celebrate important milestones in the patient's smoking cessation journey, such as the first smoke-free week or month. They can receive bonus points and rewards for their achievements.

Tobacco-Free Community: Establish a supportive community where patients can connect with others who have successfully quit smoking or are on their quitting journey. They can share experiences, offer encouragement, and receive motivation.

Quit Smoking Resources: Provide patients with educational modules and resources on quitting smoking and managing tobacco cravings. They can earn points for completing these modules and gaining knowledge.

Quit Smoking Journal: Encourage patients to maintain a quit smoking journal to document their experiences, cravings, and reflections. They can earn points for regularly updating their journal.

Social Accountability: Incorporate a social element where patients can publicly declare their commitment to quit smoking. They can earn recognition and points for staying accountable to their goal.

Regular Check-ins: Schedule regular check-ins with the patient to review their smoking cessation progress, celebrate achievements, and adjust the gamification plan based on their feedback and needs.

By implementing this gamification plan, the patient can be motivated to quit smoking and embrace a tobacco-free lifestyle. The gamified approach can make their smoking cessation journey engaging and rewarding, leading to sustained positive changes over time.

Creating a gamification plan to help the patient make progress over time on point 11 (Alcohol Consumption-Moderation or Cessation) can encourage them to manage their alcohol intake and adopt a healthier lifestyle. Gamification can make the process enjoyable and motivating, fostering adherence to alcohol management strategies. Here's a gamification plan:

Drink Smart Challenge: Develop a “Drink Smart Challenge” where the patient sets specific goals related to alcohol consumption, such as reducing the number of drinks per week or achieving alcohol-free days. They can earn points for achieving these goals.

Alcohol Tracker: Implement a tracker to monitor the patient's alcohol intake regularly. Patients can earn rewards for meeting milestones, such as alcohol-free days or weeks.

Mocktail Creativity Quests: Encourage patients to create and share alcohol-free mocktail recipes.

They can earn points for trying out new mocktails and sharing their favorites.

Physical Activity to Distract from Drinking: Create physical activity challenges aimed at providing alternative activities to drinking. Patients can earn points for engaging in activities like sports, hiking, or yoga.

Stress Relief Activities: Offer stress-relief quests and challenges, as stress can be a trigger for excessive alcohol consumption. Patients can earn points for practicing relaxation techniques, mindfulness, or meditation.

Alcohol Reduction Support: Provide support and rewards for patients aiming to reduce their alcohol consumption or transition to moderation. Encourage them to celebrate their progress.

Medication and Treatment Adherence Rewards: If the patient is using medications or treatments to manage alcohol consumption, reward them for adhering to their treatment plan and following their healthcare provider's recommendations.

Benefits of Moderation Insights: Use a mobile app or platform to provide health insights related to the benefits of moderate alcohol consumption. Patients can earn points for reviewing and acknowledging these positive changes.

Virtual Coaching Support: Offer virtual coaching sessions with healthcare professionals specializing in alcohol management. Patients can earn points for attending coaching sessions.

Alcohol-Free Milestone Celebrations: Celebrate important milestones in the patient's alcohol management journey, such as achieving alcohol moderation or cessation. They can receive bonus points and rewards for their achievements.

Alcohol-Free Community: Establish a supportive community where patients can connect with others who are managing their alcohol intake. They can share experiences, offer encouragement, and receive motivation.

Quit Drinking Resources: Provide patients with educational modules and resources on alcohol moderation or cessation. They can earn points for completing these modules and gaining knowledge.

Alcohol Management Journal: Encourage patients to maintain a journal to document their alcohol management experiences, cravings, and reflections. They can earn points for regularly updating their journal.

Social Accountability: Incorporate a social element where patients can publicly declare their commitment to alcohol moderation or cessation. They can earn recognition and points for staying accountable to their goal.

Regular Check-ins: Schedule regular check-ins with the patient to review their alcohol management progress, celebrate achievements, and adjust the gamification plan based on their feedback and needs.

By implementing this gamification plan, the patient can be motivated to manage their alcohol intake and embrace a healthier lifestyle. The gamified approach can make their alcohol management journey engaging and rewarding, leading to sustained positive changes over time.

Creating a gamification plan to help the patient make progress over time on point 12 (Substance Abuse or Addiction) can encourage them to seek support and adopt healthier habits to overcome addiction. Gamification can make the process enjoyable and motivating, fostering adherence to addiction management strategies. It's essential to note that addiction is a complex issue, and gamification alone may not be sufficient for treatment. Here's a gamification plan with a focus on seeking help and making positive changes:

Support Seeker Challenge: Develop a “Support Seeker Challenge” where the patient sets goals related to seeking professional help or joining a support group. They can earn points for taking steps toward addressing their addiction.

Sobriety Tracker: Implement a tracker to monitor the patient's progress in achieving sobriety. Patients can earn rewards for meeting milestones, such as sober days or weeks.

Healthy Coping Strategies Quests: Encourage patients to explore and practice healthy coping strategies that can replace substance use. They can earn points for trying out new coping techniques.

Physical Activity for Mental Well-being: Create physical activity challenges aimed at supporting mental well-being during recovery. Patients can earn points for engaging in activities like yoga, meditation, or nature walks.

Stress Relief Activities: Offer stress-relief quests and challenges, as stress can be a trigger for substance abuse. Patients can earn points for practicing relaxation techniques, mindfulness, or deep breathing exercises.

Medication and Treatment Adherence Rewards: If the patient is using medications or treatments for addiction management, reward them for adhering to their treatment plan and following their healthcare provider's recommendations.

Benefits of Sobriety Insights: Use a mobile app or platform to provide insights related to the benefits of sobriety. Patients can earn points for reviewing and acknowledging these positive changes.

Virtual Counseling Support: Offer virtual counseling sessions with addiction specialists or therapists.

Patients can earn points for attending counseling sessions.

Recovery Milestone Celebrations: Celebrate important milestones in the patient's recovery journey, such as achieving a specific period of sobriety. They can receive bonus points and rewards for their achievements.

Recovery Community: Establish a supportive community where patients can connect with others in recovery. They can share experiences, offer encouragement, and receive motivation.

Recovery Resources: Provide patients with educational modules and resources on addiction recovery.

They can earn points for completing these modules and gaining knowledge.

Recovery Journal: Encourage patients to maintain a journal to document their recovery journey, challenges, and reflections. They can earn points for regularly updating their journal.

Social Accountability: Incorporate a social element where patients can share their commitment to recovery publicly. They can earn recognition and points for staying accountable to their goals.

Regular Check-ins: Schedule regular check-ins with the patient to review their recovery progress, celebrate achievements, and adjust the gamification plan based on their feedback and needs.

Motivational Rewards: Provide motivational rewards, such as inspiring quotes or affirmations, to uplift patients during their recovery journey. They can earn points for reviewing these rewards.

By implementing this gamification plan, the patient can be motivated to seek support and make positive changes in their journey towards recovery from addiction. However, it's crucial to remember that addiction treatment often requires comprehensive and personalized approaches, including professional counseling and support groups. Gamification can be a valuable complementary tool to support the recovery process, but it should be integrated into a holistic treatment plan.

### Gamification

In another implementation, a comprehensive gamification plan to help the patient make progress over time on points 1 to 12 (Diabetes Risk Factors and Various Medical Conditions) can provide a holistic approach to their overall health and well-being. By incorporating gamification elements across multiple areas, the patient can stay engaged, motivated, and focused on achieving positive changes.

### Here's a Multi-Faceted Gamification Plan:

Health Hero Avatar: Create a personalized health hero avatar that represents the patient's journey. As the patient progresses and makes positive changes, the avatar can level up and unlock new features.

The avatar could be a friendly and relatable animated character representing the patient's “Health Hero.” The avatar will guide the patient through their health journey, offering encouragement and support. As the patient makes progress on each objective, the avatar's appearance could evolve, gaining new features and accessories.

Health Quest Board: Implement a virtual quest board with different challenges related to each of the 12 points. The patient can choose quests based on their interests and priorities. The user interface could feature an interactive health dashboard, displaying the patient's progress on each of the 12 points. Colorful charts and graphs can visually represent their achievements and areas for improvement. Gamification aspects like progress bars and level indicators can encourage the patient to keep working towards their health goals.

Daily Health Score: Assign a daily health score based on the patient's progress in each area. They can earn points for completing tasks related to exercise, nutrition, stress reduction, and other aspects of their health.

Leveling and Badges: Introduce leveling up and badges for accomplishing specific milestones or maintaining consistency in various health aspects. For example, reaching a specific blood pressure target or achieving a certain number of smoke-free days can unlock badges.

Gamified Chatbot: The chatbot could be an AI-powered virtual health assistant that communicates with the patient on a weekly basis. The chatbot will engage the patient in friendly conversations, ask about their progress, and provide personalized health tips and challenges based on the objectives for points 1-12. Completing challenges can earn the patient points and virtual rewards.

Virtual Adventures: Create virtual adventures related to the patient's health goals. As they progress in different areas, they can unlock new adventures, explore different destinations, and earn rewards.

Health-Related Mini-Games: Incorporate health-related mini-games or quizzes on topics like nutrition, exercise, and stress management. Patients can earn points for correctly answering questions and applying the knowledge in their lives.

Community Challenges: Organize community-wide challenges based on different health aspects.

Patients can participate, compete, and encourage each other, fostering a sense of camaraderie.

Social Support Network: Establish a supportive online community where patients can share their progress, ask questions, and receive encouragement from peers and healthcare professionals.

Health Streak Rewards: Encourage patients to maintain health streaks in different areas, such as exercising for a certain number of consecutive days or staying smoke-free. They can earn bonus points for sustained efforts.

Virtual Health Shop: Create a virtual health shop where patients can redeem earned points for health-related rewards, such as wellness products, fitness gear, or healthy cooking ingredients.

Coaching and Expert Support: Offer virtual coaching sessions with healthcare professionals or experts in various health fields. Patients can earn points for attending coaching sessions.

Regular Check-ins and Progress Reports: Schedule regular check-ins with the patient to review their progress, celebrate achievements, and identify areas for improvement. Provide visual progress reports to motivate them further.

Quest Personalization: Allow patients to personalize their quests based on their preferences and health priorities. This individualized approach can enhance motivation and engagement.

Real-world Rewards: Partner with local businesses to offer real-world rewards for achieving specific health milestones, such as free fitness classes or healthy meal vouchers.

Gamification Tournaments: Organize occasional gamification tournaments with exciting prizes to keep patients motivated and actively participating in their health journey.

Quest-Based Interface: The user interface can be designed as a quest-based system, where each objective corresponds to a specific quest. As the patient completes quests and meets their health goals, they unlock new quests and earn experience points (XP) for their health hero avatar, encouraging continuous engagement.

Reward Chest: The interface could include a reward chest that the patient can open when they achieve significant milestones or complete multiple objectives. The chest may contain surprise rewards, such as extra points, badges, or virtual items for their avatar.

Virtual Adventure Map: The interface could present a virtual adventure map with different locations corresponding to each health objective. As the patient progresses in each area, they move along the map, exploring new locations and earning rewards unique to each destination.

Health Mini-Games: The chatbot or interface could offer interactive health mini-games related to each health objective. Completing these mini-games successfully can earn the patient bonus points and rewards.

Leaderboards and Social Interaction: The interface could include leaderboards that showcase the patient's progress compared to other users. Social interaction features, like messaging or sharing achievements, can foster a sense of competition and community support.

Quest Timer and Time-Based Rewards: Some objectives may require consistent efforts over time. The interface could feature a quest timer that rewards the patient for completing specific tasks regularly.

For example, they earn extra points for meditating or exercising daily for a certain duration.

Customizable Avatar Features: Patients can earn virtual currency or points through gamification to customize their avatar's appearance, clothing, and accessories. This personalization enhances their sense of ownership and achievement.

Daily Streak Challenges: The interface or chatbot could offer daily streak challenges for each health objective. Maintaining daily streaks can lead to streak bonuses and XP boosts for the health hero avatar.

Quest Completion Rewards: Completing all objectives for points 1-12 could trigger a special reward or achievement. Patients can receive a virtual trophy or certificate as a recognition of their commitment to their health journey.

By integrating these avatar, interface, and chatbot elements with gamification aspects, the patient can have an engaging and enjoyable health experience. The gamified approach can motivate and empower the patient to make continuous progress on their health objectives while fostering a positive and interactive user experience. The patient can be motivated to make progress across various health areas. The gamified approach can make their health journey enjoyable, rewarding, and sustainable, leading to positive changes over time.

### Goal Generation System

In one embodiment, a system is goal driven with user medical long term data and mobile fitness device short term data (HRV, BP, SpO2, temperature, activity data) and can generate weekly goals for point 1 for the patient to progress from start to desired destination. The goal-driven system described next integrates long-term medical data and short-term data from mobile fitness devices to generate personalized weekly goals for the patient to progress on point 1 (Diabetes Risk Factors) towards their desired destination. The system aims to provide a comprehensive and data-driven approach to manage the patient's health effectively.
1. Data Integration: The system gathers long-term medical data from the patient's electronic health records (EHR) or personal health records (PHR). This data includes medical history, blood glucose levels, HbA1c readings, insulin sensitivity, lipid profile, and other relevant information.

Additionally, the system collects short-term data from various mobile fitness devices. These devices may include heart rate variability (HRV) monitors, blood pressure (BP) monitors, pulse oximeters for SpO2 measurement, digital thermometers for temperature tracking, and activity trackers for physical activity data.
2. Data Analysis and Machine Learning: The system uses advanced data analysis techniques and machine learning algorithms to process the combined long-term and short-term data. It identifies patterns, trends, and potential risk factors related to diabetes and other health conditions.
3. Health Risk Assessment: Based on the data analysis, the system performs a health risk assessment for the patient, identifying areas of concern and potential risk factors associated with diabetes. It also considers the patient's overall health status, lifestyle, and health goals.
4. Personalized Goal Setting: Using the health risk assessment results, the system generates personalized weekly goals for the patient. These goals are designed to address specific diabetes risk factors and improve overall health. The goals are realistic, achievable, and tailored to the patient's current health status and capabilities.
5. Weekly Goal Plan: Each week, the system presents the patient with a detailed weekly goal plan. The plan includes specific tasks and activities related to lifestyle modifications, nutrition, physical activity, stress management, and medication adherence if necessary.
6. Data Feedback and Monitoring: As the patient progresses through the weekly goals, the system continuously monitors their short-term data from fitness devices to provide real-time feedback. The patient can view their health data, track their progress, and receive insights into their performance.
7. Gamification Aspects: To enhance patient motivation and engagement, the system incorporates gamification elements. For example:
Points and Rewards: The patient earns points for completing weekly goals and achieving specific health milestones. Accumulated points can unlock rewards or virtual items for their health avatar.

Health Hero Avatar: The patient's health avatar represents their progress on the health journey. As they complete goals, the avatar grows healthier and gains new features.

Challenges and Quests: The system may offer optional health challenges or quests related to diabetes risk factors. Completing these additional tasks earns the patient bonus points.
8. Health Coach and Support: The system can provide access to a virtual health coach or healthcare professional. The coach reviews the patient's progress, offers personalized advice, and adjusts weekly goals as needed. They can also provide motivational support and answer any health-related queries.
9. Long-term Progress Tracking: The system maintains a long-term progress tracker, enabling the patient to review their journey over time. It visualizes improvements in diabetes risk factors and overall health, reinforcing the patient's commitment to their health goals.
10. Data Privacy and Security: Given the sensitive nature of health data, the system ensures robust data privacy and security measures. It complies with healthcare regulations to protect the patient's personal information.

By integrating long-term medical data and short-term fitness device data, this goal-driven system can provide patients with personalized and data-backed weekly goals to address diabetes risk factors effectively. The gamification aspects make the health journey engaging, motivating, and rewarding, leading to sustained progress over time.

### User Driven Goal Management

To accommodate user-driven goal management, the system should empower the patient to actively participate in setting their goals based on their preferences and priorities. Here's how the system can be modified:

Goal Personalization: The system allows the patient to set their own long-term health goals, such as specific blood glucose target levels or desired changes in lifestyle habits. The patient can prioritize certain diabetes risk factors that they want to focus on first.

Flexible Goal Tracking: The system incorporates user inputs on the frequency and intensity of short-term data tracking. The patient can decide how often to measure their HRV, BP, SpO2, temperature, and activity data, based on their comfort and convenience.

Goal Adjustments: The system enables the patient to adjust their weekly goals as needed. If they find certain goals challenging or too easy, they can modify them with the guidance of the virtual health coach.

User-Initiated Feedback: The patient can initiate feedback requests from the virtual health coach or healthcare professional whenever they need advice or clarification on their progress.

User-Driven Rewards: The system allows the patient to choose personalized rewards or incentives to motivate them further. They can pick rewards that align with their interests, such as a hobby-related gift or a wellness experience.

Automated Goal Setting Using AI: Similar to how users are guided by Google Maps from start to destination, the system can use AI to automate goal setting while considering user preferences and real-time health data:

User Input and Preferences: The AI-powered system prompts the patient to provide information about their health status, lifestyle, and health goals. The patient may also share any specific diabetes risk factors they want to address first.

Data Analysis and Learning: The AI system processes the user's input and analyzes their long-term medical data, short-term fitness device data, and preferences. It uses machine learning algorithms to understand patterns and trends.

Personalized Goal Generation: Based on the user's input, health data, and preferences, the AI system generates personalized weekly goals to address the user's diabetes risk factors effectively. The goals are flexible and adaptable to the user's progress.

Real-Time Adjustment: The AI system continuously monitors the user's short-term health data from fitness devices. It adjusts the weekly goals in real-time to align with the user's current health status and capabilities.

Dynamic Guidance: Similar to Google Maps dynamically updating directions based on traffic conditions, the AI system dynamically guides the user through their health journey. It provides real-time feedback and motivates the user to stay on track.

Health Recommendations: The AI system offers health recommendations, such as reminders to take medications or encouraging the user to engage in stress-relief activities based on their HRV data.

Predictive Insights: The AI system can provide predictive insights, forecasting the user's potential health outcomes based on their progress and adherence to goals.

Natural Language Interaction: The AI-powered virtual health coach communicates with the user in a natural language interface, making the guidance more user-friendly and approachable.

Continuous Learning: The AI system learns from the user's interactions and feedback, refining its goal-setting approach to better suit the user's needs over time.

By combining user-driven goal management with AI-powered automated goal setting, the system provides a balanced approach to managing diabetes risk factors. The user actively participates in setting their health goals, while the AI provides personalized, data-driven guidance, much like a virtual health navigator. This dynamic collaboration can motivate and empower the user on their health journey, leading to improved health outcomes.

The system can collect data from a population. describe modification to above system to handle a user driven goal management and then describe an automated goal setting guided by population data.

To allow user-driven goal management while collecting data from a population, the system can be work as follows:

User Goal Selection: Users are provided with the option to select from a range of predefined health goals based on diabetes risk factors and common health objectives. They can also input their own custom goals if desired.

Goal Comparison: The system compares the user's chosen goals with data from the population to provide context and insights. Users can see how their selected goals align with the average or recommended goals of similar individuals.

Peer Group Comparisons: Users have the option to join peer groups with similar health profiles and goals. They can share progress, compare achievements, and support one another through the system's community features.

Goal Progress Dashboard: The user has access to a personalized goal progress dashboard that showcases how they are faring compared to their chosen goals and the progress of the population as a whole.

User-Driven Data Sharing: Users can opt-in to share anonymized data from their health goals and progress. This contributes to the population data and helps in generating insights for other users.

Automated Goal Setting Guided by Population Data: To guide automated goal setting using population data, the system can leverage artificial intelligence and aggregate data from multiple users to generate personalized goals for new users. Here's how it can be done:

Data Aggregation: The system collects anonymized data from the population, including long-term medical data, short-term fitness device data, and user-driven goal progress.

Data Analysis and Pattern Recognition: Advanced data analysis and machine learning algorithms are used to identify patterns and trends within the aggregated population data. The system detects relationships between certain health metrics and successful goal achievements.

Goal Recommendation Engine: The AI-powered goal recommendation engine uses the population data insights to suggest personalized goals for new users. These goals are based on the achievements of similar individuals with comparable health profiles.

Dynamic Goal Adjustments: As new users progress towards their goals and share data, the AI system continuously updates and refines the goal recommendations. This dynamic approach adapts to changes in the population's health outcomes.

Customized Goal Options: The system provides multiple goal options for users based on the collective achievements of the population. Users can choose from the recommended goals or set their own based on the insights provided.

Population Benchmarking: The AI system benchmarks users' progress against the population's average or top performers, motivating users to strive for better health outcomes.

Longitudinal Insights: Over time, the system gains more data, enabling it to identify long-term trends and success factors related to specific health goals. This information informs future recommendations for new users.

User Feedback Loop: Users' progress and feedback are continuously integrated into the system's learning process, contributing to the improvement of goal-setting algorithms.

By combining user-driven goal management with automated goal setting guided by population data, the system offers a powerful tool for personalized health management. Users can benefit from insights drawn from a diverse population, and the system can continuously refine its recommendations based on real-world data from a broad range of users. This combination enhances the user experience and improves the overall effectiveness of the health goal setting process.

The goal recommendation engine is a crucial component of the system that leverages artificial intelligence and machine learning algorithms to provide personalized and data-driven health goals to users. It utilizes aggregated population data and individual user data to generate goal recommendations that align with each user's health profile, preferences, and progress. Here's a detailed description of the goal recommendation engine:

### 1. Data Collection and Aggregation:

The recommendation engine collects anonymized health data from the population, including long-term medical records, short-term fitness device data (HRV, BP, SpO2, temperature, activity), and user-driven goal progress. The data is securely aggregated and stored in a centralized database.

### 2. Data Preprocessing:

Before analyzing the data, the engine preprocesses it to clean and transform it into a consistent format. Missing or incomplete data points are handled, and any noisy data is filtered out to ensure data quality.

### 3. Data Analysis and Pattern Recognition:

The engine uses advanced data analysis techniques and machine learning algorithms to identify patterns and trends within the aggregated population data. It examines the correlations between different health metrics and successful goal achievements.

### 4. User Profiling:

When a new user joins the system, the recommendation engine creates a personalized health profile based on the user's input, long-term medical data, and short-term fitness device data. The profile includes health history, risk factors, lifestyle preferences, and current health status.

### 5. Similarity and Clustering:

The recommendation engine employs similarity and clustering algorithms to group users with similar health profiles and progress. Users are placed into clusters based on their health characteristics and goals.

### 6. Goal Recommender System:

Using the identified clusters and population data insights, the goal recommender system generates personalized goal recommendations for each user. The system takes into account the user's current health status, desired improvements, and the achievements of similar users in the population.

### 7. Goal Diversity and Flexibility:

The recommendation engine provides users with a diverse set of goal options tailored to their health profile. Users can choose from a range of goals or set their own customized goals, taking into consideration their preferences and capabilities.

### 8. Dynamic Goal Adjustment:

As users progress towards their goals and share their data, the recommendation engine continuously monitors and updates the goal recommendations. The system adapts to changes in the population's health outcomes and refines its suggestions accordingly.

### 9. Benchmarking and Insights:

The engine benchmarks each user's progress against the population's average or top-performing individuals with similar health profiles. It provides insights and comparisons to motivate users to improve their health outcomes.

### 10. User Feedback Integration:

The recommendation engine incorporates user feedback and progress data into its learning process. As users achieve their goals or encounter challenges, the engine learns from their experiences to enhance future goal recommendations.

### 11. Real-Time Goal Tracking:

The engine tracks users' progress towards their recommended goals in real-time, providing feedback, insights, and reminders to keep them on track.

### 12. Privacy and Data Security:

The recommendation engine adheres to strict privacy regulations and ensures that all user data is anonymized and securely stored. Only aggregated data is used for generating goal recommendations.

By leveraging advanced data analysis, machine learning, and user-centric approaches, the goal recommendation engine delivers personalized and effective health goals to users. The engine's continuous learning and real-time adaptability make it a valuable tool in guiding users towards improved health outcomes while respecting their individual preferences and progress.

The method includes determining food or drink intake, predicting glucose level over a predetermined period, analyzing user data from the wearable device and applying machine learning algorithms to identify individual behavior pattern, and generating a CBT intervention to keep glucose level within a medically safe range.
function predict_glucose_level(food_or_drink_data):
   #Use machine learning model to predict glucose level based on food or drink intake data
   predicted_glucose_level=ML_model(food_or_drink_data)
   return predicted_glucose_level
function generate_cbti_for_glucose_control(predicted_glucose_level):

```
  if predicted_glucose_level > medically_safe_upper_limit:
    # Generate Cognitive Behavioral Therapy (CBT) intervention for high glucose levels
    cbt_intervention = generate_high_glucose_cbt_intervention(predicted_glucose_level)
  elif predicted_glucose_level < medically_safe_lower_limit:
    # Generate Cognitive Behavioral Therapy (CBT) intervention for low glucose levels
    cbt_intervention = generate_low_glucose_cbt_intervention(predicted_glucose_level)
  else:
    # Generate Cognitive Behavioral Therapy (CBT) intervention for maintaining glucose levels
within a safe range
    cbt_intervention = generate_normal_glucose_cbt_intervention(predicted_glucose_level)
  return cbt_intervention
```

#Main function for processing food or drink intake data and generating CBT interventions
function main(food_or_drink_data):
   glucose_prediction=predict_glucose_level(food_or_drink_data)
   cbt_intervention=generate_cbti_for_glucose_control(glucose_prediction)
   return cbt_intervention

The method includes determining a user stress, predicting glucose level over a predetermined period from the user stress, analyzing user data from the wearable device and applying machine learning algorithms to identify individual behavior pattern, and generating a CBT intervention to keep glucose level within a medically safe range.
function predict_glucose_level_from_stress(stress_data):
   #Use machine learning model to predict glucose level based on stress data
   predicted_glucose_level=ML_model(stress_data)
   return predicted_glucose_level

The method includes determining a user sleep condition with wearable device monitoring, predicting glucose level over a predetermined period from a lack of sleep, analyzing user data from the wearable device and applying machine learning algorithms to identify individual behavior pattern, and generating a CBT intervention to keep glucose level within a medically safe range.
function predict_glucose_level_from_sleep(sleep_data):
   #Use machine learning model to predict glucose level based on sleep data
   predicted_glucose_level=ML_model(sleep_data)
   return predicted_glucose_level

The medical condition comprises hypertension, and the factors comprise genetics, obesity and weight, physical activity, diet, ethnicity, blood pressure, smoking, and stress factors.
function generate_cbt_intervention_for_hypertension(user_data):
   #Use machine learning algorithms to identify individual behavior patterns related to hypertension
   behavior_patterns=ML_identify_behavior_patterns(user_data)
   #Generate tailored CBT interventions based on identified behavior patterns
   cbt_intervention=generate_tailored_hypertension_cbt_intervention(behavior_patterns)
   return cbt_intervention
#Main function for processing user data and generating tailored CBT interventions for hypertension function main(user_data):
   cbt_intervention=generate_cbt_intervention_for_hypertension(user_data)
   return cbt_intervention

The method includes analyzing user data from the wearable device and applying machine learning algorithms to identify individual behavior patterns, and generating tailored CBT interventions.

Similar operations can be done to correct hypertension conditions detected by the system via smart watch detection of blood pressure.

In addition to real-time course corrections detailed above, the system can adjust the intermediate and long term goals. The dynamic goal adjustment engine is a critical component of the system that continuously monitors users' progress and adapts their goals in real-time to ensure they remain relevant and achievable. This engine leverages artificial intelligence, machine learning, and user data to fine-tune and optimize users' goals based on their changing health status and performance. Here's a detailed description of the dynamic goal adjustment engine:

### 1. Real-Time Data Integration:

The dynamic goal adjustment engine continuously receives and integrates real-time data from users' mobile fitness devices, health apps, and other connected devices. This data includes short-term health metrics like HRV, BP, SpO2, temperature, and activity levels.

### 2. User Progress Tracking:

The engine tracks users' progress towards their assigned goals on an ongoing basis. It evaluates how well users are meeting their target metrics and achieving their desired outcomes.

### 3. Goal Performance Analysis:

Using machine learning algorithms, the engine analyzes historical user data to assess the effectiveness of previously assigned goals. It identifies patterns and trends that correlate with successful goal attainment.

### 4. Personalized Goal Evaluation:

Based on the analysis of real-time data and historical performance, the engine evaluates the effectiveness of each user's current goals in light of their health profile and progress.

### 5. Dynamic Goal Adaptation:

If the engine detects that a user is making significant progress or struggling to achieve their goals, it dynamically adjusts the goals accordingly. It takes into account the user's health trajectory and ensures that goals remain challenging but realistic.

### 6. Goal Graduation and Complexity:

The dynamic goal adjustment engine allows for goal graduation. As users achieve certain milestones or demonstrate consistent progress, it can increase the complexity or intensity of their goals to provide continued challenges.

### 7. Flexibility and User Input:

The engine takes into account users' feedback and input on their goals. Users can provide insights into their experience, capabilities, and preferences, which the engine considers when adjusting their goals.

### 8. Risk Assessment and Safety:

The dynamic goal adjustment engine also performs real-time risk assessments to ensure that users' goals are safe and appropriate for their health conditions. It takes into account factors such as potential risks of overexertion or rapid changes in health metrics.

### 9. Long-Term Health Trajectory:

The engine considers the long-term health trajectory of each user when making adjustments to their goals. It aims to guide users towards sustainable progress that aligns with their overall health objectives.

### 10. Communication and Feedback:

The engine communicates goal adjustments to users in an understandable and motivational manner. It provides feedback on users' achievements and encourages them to stay on track.

### 11. Data Privacy and Security:

The dynamic goal adjustment engine ensures the privacy and security of users' real-time health data. It complies with regulations and employs encryption to protect sensitive information.

### 12. Learning and Improvement:

As the dynamic goal adjustment engine continuously processes user data, it learns from the feedback and outcomes to improve its goal-setting algorithms. This iterative process enhances the accuracy and effectiveness of goal adjustments over time.

By incorporating real-time data analysis, user feedback, and continuous learning, the dynamic goal adjustment engine provides users with personalized and adaptable health goals. The engine's ability to monitor users' progress and respond dynamically to their changing needs ensures that their health journey remains effective, engaging, and aligned with their individual capabilities and aspirations. Population benchmarking in the system involves comparing an individual user's health progress against the average or top-performing users within specific clusters or groups with similar health profiles. This process serves as a motivational tool, encouraging users to strive for better health outcomes and providing them with valuable insights into their progress. Here's how the population benchmarking works:
1. User Clustering: The system utilizes clustering algorithms to group users with similar health profiles and goals. Users within a cluster share common health characteristics, risk factors, and health objectives.
2. Aggregated Population Data: For each cluster, the system aggregates anonymized data from all users within that group. This includes long-term medical data, short-term fitness device data, and individual goal progress.
3. Calculating Average Metrics: The system calculates average health metrics, such as blood glucose levels, HbA1c readings, blood pressure, HRV, SpO2, temperature, activity levels, and other relevant health indicators, based on the data from the users in each cluster.
4. Identifying Top Performers: In addition to calculating average metrics, the system identifies users within each cluster who have achieved exceptional health outcomes or made significant progress towards their goals. These users are considered top performers.
5. Benchmarking User Progress: The system compares an individual user's health metrics and goal progress against both the average metrics and the top performers' metrics within their respective cluster.
6. Providing Insights and Comparisons: Based on the benchmarking results, the system generates insights and comparisons for the individual user. Users can see how their health metrics and goal progress measure up against both the average and top-performing users in their cluster.
7. Motivational Feedback: The system provides motivational feedback to users, highlighting their achievements and areas where they can improve. Users receive positive reinforcement for progress that surpasses the cluster average and encouragement to work towards achieving outcomes similar to top performers.
8. Goal Adjustments and Recommendations: If the user's health metrics are significantly below the average or top performers, the dynamic goal adjustment engine may suggest adapting their goals to align with achievable improvements. Similarly, if a user is already performing at an exceptional level, the system might present more challenging goals to support continued progress.
9. Community Engagement: The system may offer community features that allow users to interact and share their experiences with others within their cluster. This fosters a sense of camaraderie, motivation, and support among users with similar health profiles.
10. Longitudinal Tracking: Over time, the system tracks users' progress longitudinally, providing them with trend analysis to show their journey towards improved health outcomes. This tracking helps users understand the impact of their efforts and celebrate their achievements.
11. Data Anonymization and Privacy: Population benchmarking is conducted using aggregated and anonymized data, ensuring individual users' privacy and compliance with data protection regulations. Through population benchmarking, users gain a clearer understanding of their health progress in relation to their peers. This sense of context and comparison serves as a powerful motivational tool, encouraging users to stay committed to their health goals and strive for better outcomes. The insights provided by the benchmarking process enable users to make informed decisions, leading to more effective and personalized health management.

Longitudinal insights refer to the analysis and tracking of users' health data and progress over an extended period, allowing for a comprehensive understanding of their health journey over time. This process provides valuable insights into trends, patterns, and improvements in users' health metrics and goal achievements. Here's a detailed description of how longitudinal insights work:
1. Data Collection and Integration: The system continuously collects and integrates users' health data over time. This includes long-term medical records, short-term fitness device data, and user-driven goal progress. The data is securely stored and updated in the users' profiles.
2. Historical Health Snapshot: At the beginning of a user's engagement with the system, a historical health snapshot is created. This snapshot represents the user's initial health status, which includes relevant medical history, baseline health metrics, and any pre-existing health conditions.
3. Ongoing Data Analysis: As users continue to interact with the system, the longitudinal insights engine analyzes their health data at regular intervals. It applies data analysis techniques and machine learning algorithms to identify trends and patterns in the data.
4. Health Metric Trends: The engine examines trends in health metrics such as blood glucose levels, HbA1c readings, blood pressure, HRV, SpO2, temperature, activity levels, and other relevant indicators. It identifies whether these metrics are improving, stable, or showing signs of deterioration over time.
5. Goal Progress Tracking: The engine tracks users' progress towards their assigned health goals over time. It assesses how well users are meeting their target objectives and making improvements towards desired health outcomes.
6. Long-Term Risk Assessment: Based on the longitudinal data analysis, the engine performs long-term risk assessments. It evaluates the user's risk factors for certain health conditions, identifies potential areas of concern, and predicts future health outcomes based on their progress.
7. Insights Visualization: The system presents longitudinal insights in the form of easy-to-understand visualizations. Users can view graphs, charts, and trend lines illustrating their health metrics and goal progress over time.
8. Milestone and Achievement Tracking: The engine identifies and highlights significant milestones and achievements in the user's health journey. This could include reaching target health metrics, maintaining consistent progress, or successfully managing specific risk factors.
9. User Empowerment: Longitudinal insights empower users with a deeper understanding of their health progress. Users can gain insights into the effectiveness of their efforts, identify factors that positively influence their health, and make informed decisions regarding their health goals and lifestyle choices.
10. Personalized Recommendations: The engine may offer personalized recommendations based on the longitudinal insights. These recommendations may include adjustments to health goals, lifestyle modifications, or additional health interventions to support the user's ongoing progress.
11. Continuous Learning and Improvement: The longitudinal insights engine continuously learns from users' data and feedback, improving its analysis and recommendations over time. It adapts to changes in user behavior and provides increasingly relevant insights.

Through longitudinal insights, users can track the trajectory of their health improvements, celebrate their successes, and address any challenges they may encounter. The continuous monitoring and analysis of users' health data provide valuable feedback that fosters a proactive approach to health management. As users gain awareness of the impact of their actions on their health, they are better equipped to make informed decisions and stay committed to their long-term health goals.

Performance improvement is achieved when millions of smartwatches stream data to the system for Cognitive Behavioral Therapy (CBT) application by employing several strategies that ensure reliability, scalability, and efficiency:
Data Processing and Analysis: Implementing efficient data processing and analysis pipelines to handle the influx of data from millions of smartwatches. This may involve using distributed computing frameworks, such as Apache Spark or Apache Flink, to parallelize data processing tasks and handle large-scale data streams.
Distributed Database and Storage: Utilizing distributed databases, like Apache Cassandra or Amazon DynamoDB, to store and manage the vast amount of streaming data reliably and efficiently. This ensures data availability, fault tolerance, and scalability.
Load Balancing and Horizontal Scaling: Employing load balancing techniques and horizontal scaling across multiple servers or cloud instances to distribute the incoming data stream evenly and ensure that the system can handle the increasing load as the number of smartwatches grows.
Real-time Stream Processing: Implementing real-time stream processing using technologies like Apache Kafka or Apache Flink to analyze data as it arrives, enabling immediate feedback and actionable insights for users.
Microservices Architecture: Adopting a microservices architecture, where different components of the system are decoupled and can be independently scaled, allowing for targeted resource allocation based on specific needs.
Caching and In-Memory Databases: Utilizing caching mechanisms and in-memory databases to store frequently accessed data and reduce the need for repeated computations, thereby improving response times.
Optimized Algorithms: Using optimized algorithms for data analysis and processing to reduce computation time and improve the overall efficiency of the system.
Asynchronous Processing: Implementing asynchronous processing for non-time-critical tasks, allowing the system to handle large volumes of data without blocking other operations.
Data Compression and Aggregation: Implementing data compression and aggregation techniques to minimize the amount of data transmitted and stored, reducing the overall system overhead.
Monitoring and Performance Tuning: Continuously monitoring system performance, identifying bottlenecks, and fine-tuning the system to ensure optimal resource utilization and responsiveness.

By combining these strategies, the system can reliably and scalably handle data from millions of smartwatches streaming to the CBT application. It ensures that data is processed efficiently, real-time insights are provided, and users receive personalized feedback and interventions, contributing to improved mental health and well-being on a large scale. One of the advantages of the system is the ability to use a common set of digital therapeutics programs to provide unique, adaptive, personalized interventions for a wide variety of users. The system may also provide real-time or near-real-time responsiveness to adjust the interventions for a user's current status and situation. Applying the concept of longitudinal insights to the networked system described above enhances its ability to create and manage dynamic, individualized care plans for avoiding and managing diabetes while minimizing injections. Here's how the system can leverage longitudinal insights to optimize its digital therapeutics programs and interventions:
1. Longitudinal Health Tracking: The system collects and integrates users' health data over time, including long-term medical records, short-term fitness device data, and goal progress. This longitudinal health tracking allows the system to create a comprehensive understanding of each user's health journey and risk factors.
2. Personalized Care Plans: Using the longitudinal insights, the system tailors personalized care plans for each user, taking into consideration their unique health history, risk levels, and specific diabetes management needs. These plans may include lifestyle modifications, medication adjustments, and digital therapeutics interventions.
3. Dynamic Care Plan Adjustments: The system continuously monitors users' health metrics and progress, and it dynamically adjusts their care plans in real-time or near-real-time to reflect their current status and situation. This responsiveness ensures that the interventions remain contextually relevant and effective.
4. Predictive Risk Mitigation: Leveraging machine learning techniques, the system predicts each user's unique risk levels for diabetes-related complications. By proactively identifying potential risks, the system can take predictive actions to mitigate those risks before they escalate.
5. Adaptive Digital Therapeutics Programs: The system utilizes a common set of digital therapeutics programs, but it adapts and customizes them for different categories of patients based on their longitudinal health data and unique risk profiles. This adaptive approach optimizes intervention effectiveness for diverse user populations.
6. Coordinated Digital Therapeutics Programs: The system coordinates and manages digital therapeutics programs across various aspects of users' wellbeing. It considers the interactions between different interventions and adjusts care plans to provide comprehensive support while avoiding potential conflicts.
7. Interactions with External Devices and Users: The system interacts not only with the user whose health and wellbeing it supports but also with other devices and users, such as friends, family members, medical service providers, and other stakeholders. This holistic approach fosters a supportive network around the user's health management.
8. Minimizing Injections and Medication Management: Based on longitudinal insights, the system identifies opportunities to minimize injections through personalized diabetes management approaches. It may recommend non-invasive interventions, lifestyle modifications, or alternative medications to optimize diabetes care.
9. Continuous Learning and Improvement: The system continuously learns from user data, interactions, and outcomes, refining its interventions and care plans over time. This iterative process enhances the system's ability to deliver better health outcomes for users.
10. Real-Time Responsiveness: Longitudinal insights enable the system to provide real-time or near-real-time responsiveness to users' changing health needs. Users receive contextually relevant interventions and support as their health status evolves.

By leveraging longitudinal insights, the networked system optimizes its ability to deliver adaptive, personalized interventions for a wide range of users managing diabetes or avoiding its onset. The system's data-driven approach ensures that care plans are tailored to each user's needs, promoting better diabetes management and overall health outcomes.

In some implementations, a networked system of devices creates and manages dynamic, individualized care plans for avoiding diabetes and how to best manage diabetes if unavoidable and to minimize injections if possible. The system can coordinate digital therapeutics programs across many different aspects of users' wellbeing, taking into consideration interactions, risks, and personalized behavior that would not be apparent to a physician. This allows the system to interact with various other systems to automatically adjust the content of each user's care plan. The system also carries out the plans by administering real-time digital therapeutics that are provided through user devices. The system can react to each user's current situation with contextually relevant interventions. The interactions of the system are not limited to a user whose health and wellness the system supports.

Rather, the system can interact with devices of other users, such as friends, family members, medical service providers, and others to support a user's health. The system can also assess unique risk levels for each patient and predictively act to mitigate those risks. Further, the system can use machine learning techniques to identify the combinations of interventions that best improve outcomes for different categories of patients.

One of the advantages of the system is the ability to use a common set of digital therapeutics programs to provide unique, adaptive, personalized interventions for a wide variety of users. The system may also provide real-time or near-real-time responsiveness to adjust the interventions for a user's current status and situation.

Applying the networked system's capabilities to points 1-12 above enhances its ability to create dynamic, individualized care plans for avoiding and managing diabetes while minimizing injections. Here's how the system can utilize its features to address each of the 12 points:

### 1. Risk Identification and Personalized Goal Setting:

The system employs longitudinal insights and machine learning to assess each user's unique risk factors for developing diabetes. Based on this information, it sets personalized goals to address specific risk factors, such as weight management, blood glucose control, and physical activity.

### 2. Gamified Approach to Lifestyle Changes:

To encourage users to make lifestyle changes, the system utilizes gamification techniques. Users can track their progress in real-time, earn rewards, and engage in challenges with their peers, leveraging the power of social interaction and motivation.

### 3. Nutritional Guidance and Meal Planning:

Using personalized data, the system provides nutritional guidance and meal planning suggestions that align with each user's health goals, preferences, and dietary restrictions.

### 4. Fitness and Exercise Routines:

The system offers dynamic exercise routines that adapt to users' progress and fitness levels, taking into account their real-time fitness data from wearable devices. It encourages physical activity and helps users maintain an active lifestyle.

### 5. Stress Management and Mindfulness Interventions:

The system incorporates stress management and mindfulness interventions, leveraging users' HRV and activity data. It provides contextually relevant mindfulness exercises to help users cope with stress.

### 6. Sleep Improvement Strategies:

By analyzing sleep data from wearable devices, the system identifies sleep patterns and disturbances. It offers personalized sleep improvement strategies and helps users establish healthier sleep habits.

### 7. Medication Adherence and Reminders:

The system provides real-time medication reminders and adherence tracking to help users manage their medications effectively. It can also predictively act on medication interactions or potential side effects.

### 8. Regular Blood Glucose Monitoring:

The system encourages users to monitor their blood glucose regularly through connected devices. It offers feedback and reminders based on longitudinal trends to help users maintain stable blood glucose levels.

### 9. Individualized Diabetes Risk Alerts:

Leveraging machine learning, the system assesses each user's unique diabetes risk factors and provides predictive risk alerts. It prompts users to take proactive measures to mitigate identified risks.

### 10. Gamified Progress Tracking:

The system gamifies progress tracking, allowing users to visualize their improvements and receive rewards for achieving milestones. The progress tracking considers both short-term fitness device data and long-term medical records.

### 11. Longitudinal Goal Adjustments:

Using longitudinal insights, the system continuously adapts users' care plans and goals to reflect their changing health status and progress. It ensures that the interventions remain relevant and effective over time.

### 12. Adaptive Interventions for Different Patient Categories:

The system uses machine learning to identify combinations of interventions that yield the best outcomes for different patient categories. It tailors care plans to suit the unique needs of various user groups based on their health data and risk profiles.

By leveraging its networked capabilities, real-time responsiveness, and adaptive interventions, the system offers a comprehensive approach to diabetes prevention and management. It provides individualized care plans that dynamically adjust based on users' evolving health data, promoting better health outcomes and overall wellness. The system's ability to interact with various devices and users, along with its data-driven approach, ensures a personalized, contextually relevant, and holistic user experience.

In one exemplary implementation, the software surveys user which problems (weight loss, smoking, drinking, drugs, anxiety, depression, pain, physical activity, nutrition, psychosocial, fatigue, sleep disorder, dysfunctional sex, treatment/drug adherence, etc) the user wishes to prioritize to solve, then traverses in order each prioritized point as the software asks user to select a series of desired sub-goals or challenges for each week and generates the action items and gamifies the challenges to encourage user to complete the sub-goals. Pseudo-code is as follows:

```
# Define the list of priority problems
priority_problems = [‘Weight Loss’, ‘Smoking’, ‘Drinking’, ‘Drugs’, ‘Anxiety’, ‘Depression’, ‘Pain’,
‘Physical Activity’, ‘Nutrition’, ‘Psychosocial’, ‘Fatigue’, ‘Sleep Disorder’, ‘Dysfunctional Sex’,
‘Treatment/Drug Adherence’]
# Initialize an empty list to store the user's prioritized problems
user_priorities = [ ]
# Survey the user on their priority problems
print(“Please select your top priority problems from the following list:”)
for i, problem in enumerate(priority_problems):
  print(f“{i+1}. {problem}”)
  answer = input(“Do you wish to prioritize this problem? (yes/no): ”)
  if answer.lower( ) == ‘yes’:
    user_priorities.append(problem)
# Initialize a dictionary to store sub-goals for each prioritized problem
sub_goals = { }
# For each prioritized problem, ask the user to select sub-goals for each week
for problem in user_priorities:
  sub_goals [problem] = [ ]
  print(f“\nSelect sub-goals for {problem}:”)
  week = 1
  while True:
    sub_goal = input(f“Week {week}: Enter a sub-goal or ‘done’ to move to the next problem: ”)
    if sub_goal.lower( ) == ‘done’:
      break
    else:
      sub_goals[problem].append(sub_goal)
      week += 1
# Initialize a dictionary to store action items for each sub-goal
action_items = { }
# For each sub-goal, generate action items and gamify the challenges
for problem, goals in sub_goals.items( ):
  action_items[problem] = { }
  print(f“\nAction items for {problem}:”)
  for i, sub_goal in enumerate(goals):
    print(f“\nSub-Goal {i+1}: {sub_goal}”)
    print(“Gamify the challenge to encourage completion:”)
    challenge = input(“Enter the gamified challenge: ”)
    action_items[problem][sub_goal] = challenge
# Display the user's prioritized problems, sub-goals, and action items
print(“\nYour Prioritized Problems:”)
for problem in user_priorities:
  print(f“- {problem}”)
print(“\nSub-Goals and Gamified Challenges:”)
for problem, goals in action_items.items( ):
  print(f“\n{problem}:”)
  for sub_goal, challenge in goals.items( ):
    print(f“ - {sub_goal}: {challenge}”)
```

One implementation uses a ThreadPoolExecutor to parallelize the process of obtaining sub-goals for each prioritized problem. The function get_sub_goals is executed concurrently for each problem, making the survey process faster. Parallelizing this part of the code can be beneficial, especially if there are many prioritized problems to handle. The implementation of parallelism may vary depending on the programming language and libraries used. Additionally, further optimization can be achieved by using more efficient data structures, as described earlier in the performance enhancement strategies. To apply performance enhancement to the pseudo code, the system optimizes data structures and parallelizes certain parts of the code using parallel programming techniques. For simplicity, parallelism for the generation of sub-goals and gamified challenges is shown below, and the implementation of parallelism depends on the programming language and platform used as follows:

```
import concurrent.futures
# Define the list of priority problems
priority_problems = [‘Weight Loss’, ‘Smoking’, ‘Drinking’, ‘Drugs’, ‘Anxiety’, ‘Depression’, ‘Pain’,
‘Physical Activity’, ‘Nutrition’, ‘Psychosocial’, ‘Fatigue’, ‘Sleep Disorder’, ‘Dysfunctional Sex’,
‘Treatment/Drug Adherence’]
# Initialize an empty list to store the user's prioritized problems
user_priorities = [ ]
# Survey the user on their priority problems
print(“Please select your top priority problems from the following list:”)
for i, problem in enumerate(priority_problems):
  print(f“{i+1}. {problem}”)
  answer = input(“Do you wish to prioritize this problem? (yes/no): ”)
  if answer.lower( ) == ‘yes’:
    user_priorities.append(problem)
# Initialize a dictionary to store sub-goals for each prioritized problem
sub_goals = { }
# Function to get sub-goals for each prioritized problem
def get_sub_goals(problem):
  goals = [ ]
  print(f“\nSelect sub-goals for {problem}:”)
  week = 1
  while True:
    sub_goal = input(f“Week {week}: Enter a sub-goal or ‘done’ to move to the next problem: ”)
    if sub_goal.lower( ) == ‘done’:
      break
    else:
      goals.append(sub_goal)
      week += 1
  return problem, goals
# Use parallelism to get sub-goals for each prioritized problem
with concurrent.futures.ThreadPoolExecutor( ) as executor:
  future_to_problem = {executor.submit(get_sub_goals, problem): problem for problem in
user_priorities}
  for future in concurrent.futures.as_completed(future_to_problem):
    problem, goals = future.result( )
    sub_goals[problem] = goals
# Initialize a dictionary to store action items for each sub-goal
action_items = { }
# For each sub-goal, generate action items and gamify the challenges
for problem, goals in sub_goals.items( ):
  action_items[problem] = { }
  print(f“\nAction items for {problem}:”)
  for i, sub_goal in enumerate(goals):
    print(f“\nSub-Goal {i+1}: {sub_goal}”)
    print(“Gamify the challenge to encourage completion:”)
    challenge = input(“Enter the gamified challenge: ”)
    action_items[problem][sub_goal] = challenge
# Display the user's prioritized problems, sub-goals, and action items
print(“\nYour Prioritized Problems:”)
for problem in user_priorities:
  print(f“- {problem}”)
print(“\nSub-Goals and Gamified Challenges:”)
for problem, goals in action items.items( ):
  print(f“\n{problem}:”)
  for sub_goal, challenge in goals.items( ):
    print(f“ - {sub_goal}: {challenge}”)
```

Advantageous data structures can be applied to the pseudo code including
1. Hash Table (Hash Map): A hash table could be used to efficiently store the user's prioritized problems, where the problem name serves as the key, and the value can be used to store any additional information related to the problem. For example, a hash table (hash map) efficiently stores the user's prioritized problems and their associated sub-goals. This data structure will allow us to perform fast retrieval and update operations. In one example, we use a hash table named sub_goals to store the sub-goals for each prioritized problem. Each key in the hash table represents a problem from the user_priorities list, and the corresponding value is a list containing the selected sub-goals for that problem. With the hash table, we can efficiently store and retrieve the sub-goals for each problem, making it easier to manage and access the data. The use of a hash table enables fast data lookup and insertion, making the surveying process more efficient and improving overall performance. Below is an example of how we can apply a hash table to the pseudo code:

```
# Define the list of priority problems
priority_problems = [‘Weight Loss’, ‘Smoking’, ‘Drinking’, ‘Drugs’, ‘Anxiety’, ‘Depression’, ‘Pain’,
‘Physical Activity’, ‘Nutrition’, ‘Psychosocial’, ‘Fatigue’, ‘Sleep Disorder’, ‘Dysfunctional Sex’,
‘Treatment/Drug Adherence’]
# Initialize an empty list to store the user's prioritized problems
user_priorities = [ ]
# Survey the user on their priority problems
print(“Please select your top priority problems from the following list:”)
for i, problem in enumerate(priority_problems):
  print(f“{i+1}. {problem}”)
  answer = input(“Do you wish to prioritize this problem? (yes/no): ”)
  if answer.lower( ) == ‘yes’:
    user_priorities.append(problem)
# Initialize a hash table to store sub-goals for each prioritized problem
sub_goals = { }
# For each prioritized problem, ask the user to select sub-goals for each week
for problem in user_priorities:
  sub_goals[problem] = [ ]
  print(f“\nSelect sub-goals for {problem}:”)
  week = 1
  while True:
    sub_goal = input(f“Week {week}: Enter a sub-goal or ‘done’ to move to the next problem: ”)
    if sub_goal.lower( ) == ‘done’:
      break
    else:
      sub_goals[problem].append(sub_goal)
      week += 1
# Display the user's prioritized problems, sub-goals, and action items
print(“\nYour Prioritized Problems:”)
for problem in user_priorities:
  print(f“- {problem}”)
print(“\nSub-Goals:”)
for problem, goals in sub_goals.items( ):
  print(f“\n{problem}:”)
  for sub_goal in goals:
    print(f“ - {sub_goal}”)
```

2. Balanced Binary Search Tree: A balanced binary search tree can be applied to store the sub-goals for each prioritized problem. This would allow for efficient insertion, deletion, and retrieval of sub-goals while keeping them sorted, if necessary.
3. Dynamic Array (Vector): Dynamic arrays can be employed to store the user's prioritized problems and their sub-goals. As the user provides input, the dynamic arrays can grow to accommodate the data without wasting memory. To apply a dynamic array (also known as a dynamic list or resizable array) to the pseudo code, we will use it to store the user's prioritized problems and their associated sub-goals. A dynamic array automatically resizes itself when needed, allowing for efficient handling of varying amounts of data. Below is an example of how we can apply a dynamic array to the pseudo code:

```
# Define the list of priority problems
priority_problems = [‘Weight Loss’, ‘Smoking’, ‘Drinking’, ‘Drugs’, ‘Anxiety’, ‘Depression’, ‘Pain’,
‘Physical Activity’, ‘Nutrition’, ‘Psychosocial’, ‘Fatigue’, ‘Sleep Disorder’, ‘Dysfunctional Sex’,
‘Treatment/Drug Adherence’]
# Initialize an empty dynamic array to store the user's prioritized problems
user_priorities = [ ]
# Survey the user on their priority problems
print(“Please select your top priority problems from the following list:”)
for i, problem in enumerate(priority_problems):
  print(f“{i+1}. {problem}”)
  answer = input(“Do you wish to prioritize this problem? (yes/no): ”)
  if answer.lower( ) == ‘yes’:
    user_priorities.append(problem)
# Initialize a dynamic array to store sub-goals for each prioritized problem
sub_goals = { }
# For each prioritized problem, ask the user to select sub-goals for each week
for problem in user_priorities:
  sub_goals[problem] = [ ]
  print(f“\nSelect sub-goals for {problem}:”)
  week = 1
  while True:
    sub_goal = input(f“Week {week}: Enter a sub-goal or ‘done’ to move to the next problem: ”)
    if sub_goal.lower( ) == ‘done’:
      break
    else:
      sub_goals[problem].append(sub_goal)
      week += 1
# Display the user's prioritized problems, sub-goals, and action items
print(“\nYour Prioritized Problems:”)
for problem in user_priorities:
  print(f“- {problem}”)
print(“\nSub-Goals:”)
for problem, goals in sub_goals.items( ):
  print(f“\n{problem}:”)
  for sub_goal in goals:
    print(f“ - {sub_goal}”)
```

In the above example, we use a dynamic array named user_priorities to store the user's prioritized problems, which automatically grows as the user selects problems. We also use another dynamic array to store the sub-goals for each prioritized problem. The dynamic array ensures that we can efficiently handle varying numbers of prioritized problems and sub-goals without wasting memory. By applying a dynamic array to the pseudo code, we improve the efficiency of data storage and manipulation, ensuring that the survey process can handle different numbers of selected problems and sub-goals effectively. The dynamic array automatically adjusts its size to accommodate the data, avoiding unnecessary memory allocations and deallocations.
4. Heap (Priority Queue): A priority queue (min-heap or max-heap) can be used to manage the prioritized problems based on their importance or urgency, making it easier to process the problems in the desired order.
5. Trie (Prefix Tree): A trie can be applied to efficiently store and search for keywords associated with sub-goals. This could be helpful in autocomplete features or when suggesting similar sub-goals to users.
6. Bloom Filter: A bloom filter can be utilized to check whether a user has already selected a particular sub-goal or not, reducing redundant data storage and improving performance. Next, a Bloom filter named sub_goal_filter to keep track of the selected sub-goals to avoid duplicates. Before adding a sub-goal to the sub_goals dictionary, we check whether it exists in the Bloom filter. If the sub-goal is not present in the Bloom filter (meaning it has not been selected before), we add it to both the dictionary and the Bloom filter. If the sub-goal is already present in the Bloom filter, we inform the user that the sub-goal has already been selected and prompt them to choose a different one. By incorporating a Bloom filter into the pseudo code, we ensure that redundant sub-goals are not added to the sub_goals dictionary, improving data storage efficiency and speeding up the survey process.

```
# Import the bloom filter library
from pybloom_live import BloomFilter
# Define the list of priority problems
priority_problems = [‘Weight Loss’, ‘Smoking’, ‘Drinking’, ‘Drugs’, ‘Anxiety’, ‘Depression’, ‘Pain’,
‘Physical Activity’, ‘Nutrition’, ‘Psychosocial’, ‘Fatigue’, ‘Sleep Disorder’, ‘Dysfunctional Sex’,
‘Treatment/Drug Adherence’]
# Initialize an empty list to store the user's prioritized problems
user_priorities = [ ]
# Survey the user on their priority problems
print(“Please select your top priority problems from the following list:”)
for i, problem in enumerate(priority_problems):
  print(f“{i+1}. {problem}”)
  answer = input(“Do you wish to prioritize this problem? (yes/no): ”)
  if answer.lower( ) == ‘yes’:
    user_priorities.append(problem)
# Initialize a Bloom filter to check selected sub-goals
sub_goal_filter = BloomFilter(capacity=1000, error_rate=0.1)
# Initialize a dictionary to store sub-goals for each prioritized problem
sub_goals = { }
# For each prioritized problem, ask the user to select sub-goals for each week
for problem in user_priorities:
  sub_goals[problem] = [ ]
  print(f“\nSelect sub-goals for {problem}:”)
  week = 1
  while True:
    sub_goal = input(f“Week {week}: Enter a sub-goal or ‘done’ to move to the next problem: ”)
    if sub_goal.lower( ) == ‘done’:
      break
    elif sub_goal not in sub_goal_filter:
      sub_goals[problem].append(sub_goal)
      sub_goal_filter.add(sub_goal)
      week += 1
    else:
      print(“Sub-goal already selected. Please choose a different one.”)
# Display the user's prioritized problems, sub-goals, and action items
print(“\nYour Prioritized Problems:”)
for problem in user_priorities:
  print(f“- {problem}”)
print(“\nSub-Goals:”)
for problem, goals in sub_goals.items( ):
  print(f“\n{problem}:”)
  for sub_goal in goals:
    print(f“ - {sub_goal}”)
```

7. Disjoint-Set (Union-Find): A disjoint-set data structure could be employed if there is a need to group sub-goals into sets based on some common characteristics or categories. To apply the disjoint-set data structure (also known as the union-find data structure) to the pseudo code, we can use it to group sub-goals into sets based on common categories. This can be helpful when users have selected similar sub-goals or when certain sub-goals share common attributes. Below is an example of how we can apply the disjoint-set data structure to the pseudo code:

```
# Define the list of priority problems
priority_problems = [‘Weight Loss’, ‘Smoking’, ‘Drinking’, ‘Drugs’, ‘Anxiety’, ‘Depression’, ‘Pain’,
‘Physical Activity’, ‘Nutrition’, ‘Psychosocial’, ‘Fatigue’, ‘Sleep Disorder’, ‘Dysfunctional Sex’,
‘Treatment/Drug Adherence’]
# Initialize an empty list to store the user's prioritized problems
user_priorities = [ ]
# Survey the user on their priority problems
print(“Please select your top priority problems from the following list:”)
for i, problem in enumerate(priority_problems):
  print(f“{i+1}. {problem}”)
  answer = input(“Do you wish to prioritize this problem? (yes/no): ”)
  if answer.lower( ) == ‘yes’:
    user_priorities.append(problem)
# Initialize a dictionary to store sub-goals for each prioritized problem
sub_goals = { }
# For each prioritized problem, ask the user to select sub-goals for each week
for problem in user_priorities:
  sub_goals[problem] = [ ]
  print(f“\nSelect sub-goals for {problem}:”)
  week = 1
  while True:
    sub_goal = input(f“Week {week}: Enter a sub-goal or ‘done’ to move to the next problem: ”)
    if sub_goal.lower( ) == ‘done’:
      break
    else:
      sub_goals[problem].append(sub_goal)
      week += 1
# Disjoint-Set (Union-Find) data structure to group sub-goals into sets based on common categories
parent = { }
rank = { }
def make_set(sub_goal):
  parent[sub_goal] = sub_goal
  rank[sub_goal] = 0
def find(sub_goal):
  if parent[sub_goal] != sub_goal:
    parent[sub_goal] = find(parent[sub_goal])
  return parent[sub_goal]
def union(sub_goal1, sub_goal2):
  root1 = find(sub_goal1)
  root2 = find(sub_goal2)
  if root1 != root2:
    if rank[root1] > rank[root2]:
      parent[root2] = root1
    else:
      parent[root1] = root2
      if rank[root1] == rank[root2]:
        rank[root2] += 1
# Group sub-goals into sets based on common categories
for problem, goals in sub_goals.items( ):
  for sub_goal in goals:
    make_set(sub_goal)
  # Assuming some sub-goals belong to the same category, we union them
  if len(goals) >= 2:
    union(goals[0], goals[1])
# Display the user's prioritized problems, sub-goals, and action items
print(“\nYour Prioritized Problems:”)
for problem in user_priorities:
  print(f“- {problem}”)
print(“\nSub-Goals:”)
for problem, goals in sub_goals.items( ):
  print(f“\n{problem}:”)
  for sub_goal in goals:
    print(f“ - {sub_goal}”)
# Display sets of sub-goals based on common categories
print(“\nSub-Goal Categories:”)
categories = { }
for goal in sub_goals:
  root = find(goal)
  if root not in categories:
    categories[root] = [ ]
  categories[root].append(goal)
for category, goals in categories.items( ):
  print(f“\nCategory: {category}”)
  for sub_goal in goals:
    print(f“ - {sub_goal}”)
```

The above code added functions to create sets, find the root of a set, and perform a union of two sets using the disjoint-set data structure. The code groups sub-goals into sets based on common categories, assuming that some sub-goals belong to the same category. By applying the disjoint-set data structure, we efficiently group similar sub-goals into sets, making it easier to manage and analyze sub-goals with shared attributes or characteristics. This can be helpful in providing personalized recommendations or insights to users based on the identified sub-goal categories.
8. Quadtree and Octree: For the sub-goals or action items with spatial attributes or hierarchical relationships, quadtrees or octrees are applied to efficiently partition and search the data. To apply a quadtree or octree to the pseudo code, we can use these spatial data structures to efficiently partition and manage sub-goals with spatial attributes or hierarchical relationships. Since the pseudo code does not have explicit spatial data, we will demonstrate a simplified example where the sub-goals are considered as 2D points with coordinates. The quadtree can be used for 2D data, and the octree for 3D data. Next, the QuadNode class is used to represent a node in the quadtree and the insert_into_quadtree function to efficiently insert sub-goals into the quadtree. The quadtree is constructed to efficiently partition the 2D space and group sub-goals based on their spatial attributes as follows:

```
# Define the list of priority problems
priority_problems = [‘Weight Loss’, ‘Smoking’, ‘Drinking’, ‘Drugs’, ‘Anxiety’, ‘Depression’, ‘Pain’,
‘Physical Activity’, ‘Nutrition’, ‘Psychosocial’, ‘Fatigue’, ‘Sleep Disorder’, ‘Dysfunctional Sex’,
‘Treatment/Drug Adherence’]
# Initialize an empty list to store the user's prioritized problems
user_priorities = [ ]
# Survey the user on their priority problems
print(“Please select your top priority problems from the following list:”)
for i, problem in enumerate(priority_problems):
  print(f“{i+1}. {problem}”)
  answer = input(“Do you wish to prioritize this problem? (yes/no): ”)
  if answer.lower( ) == ‘yes’:
    user_priorities.append(problem)
# Define a class to represent a sub-goal as a 2D point
class SubGoal:
  def _{——}init_{——}(self, x, y, name):
    self.x = x
    self.y = y
    self.name = name
# Initialize a dictionary to store sub-goals for each prioritized problem
sub_goals = { }
# For each prioritized problem, ask the user to select sub-goals (2D points) for each week
for problem in user_priorities:
  sub_goals[problem] = [ ]
  print(f“\nSelect sub-goals (2D points) for {problem}:”)
  week = 1
  while True:
    x = float(input(f“Week {week}: Enter x-coordinate or ‘done’ to move to the next problem: ”))
    if x.lower( ) == ‘done’:
      break
    y = float(input(f“Week {week}: Enter y-coordinate: ”))
    sub_goal_name = input(f“Week {week}: Enter a name for the sub-goal: ”)
    sub_goal = SubGoal(x, y, sub_goal_name)
    sub_goals[problem].append(sub_goal)
    week += 1
# Quadtree Node class
class QuadNode:
  def _{——}init_{——}(self, x, y, width, height):
    self.x = x
    self.y = y
    self.width = width
    self.height = height
    self.sub_goals = [ ]
    self.children = [None, None, None, None]
# Function to insert a sub-goal into the quadtree
def insert_into_quadtree(root, sub_goal):
  if root.children [0] is None:
    root.sub_goals.append(sub_goal)
    if len(root.sub_goals) > 1: # Split the node if it contains more than one sub-goal
      split_quad_node(root)
  else:
    index = get_quadrant_index(root, sub_goal)
    if index is not None:
      if root.children[index] is None:
        root.children[index] = QuadNode(
          root.x + (index % 2) * root.width / 2,
          root.y + (index // 2) * root.height / 2,
          root.width / 2,
          root.height / 2,
        )
      insert_into_quadtree(root.children[index], sub_goal)
# Function to get the quadrant index for a sub-goal
def get_quadrant_index(root, sub_goal):
  mid_x = root.x + root.width / 2
  mid_y = root.y + root.height / 2
  if sub_goal.x >= mid_x:
    if sub_goal.y >= mid_y:
      return 1 # Top-right quadrant
    else:
      return 3 # Bottom-right quadrant
  else:
    if sub_goal.y >= mid_y:
      return 0 # Top-left quadrant
    else:
      return 2 # Bottom-left quadrant
# Function to split a quadtree node
def split_quad_node(node):
  mid_x = node.x + node.width / 2
  mid_y = node.y + node.height / 2
  node.children[0] = QuadNode(node.x, node.y, node.width / 2, node.height / 2) # Top-left
  node.children[1] = QuadNode(mid_x, node.y, node.width / 2, node.height / 2) # Top-right
  node.children[2] = QuadNode(node.x, mid_y, node.width / 2, node.height / 2) # Bottom-left
  node.children[3] = QuadNode(mid_x, mid_y, node.width / 2, node.height / 2) # Bottom-right
# Initialize the root of the quadtree
quadtree_root = QuadNode(0, 0, 100, 100) # Assuming a 2D space with width=height=100
# Insert all sub-goals into the quadtree
for problem, goals in sub_goals.items( ):
  for sub_goal in goals:
    insert_into_quadtree(quadtree_root, sub_goal)
# Display the user's prioritized problems, sub-goals, and action items
print(“\nYour Prioritized Problems:”)
for problem in user_priorities:
  print(f“- {problem}”)
print(“\nSub-Goals:”)
for problem, goals in sub_goals.items( ):
  print(f“\n{problem}:”)
  for sub_goal in goals:
    print(f“ - {sub_goal.name} (x={sub_goal.x}, y={sub_goal.y})”)
# Display the quadtree structure
def display_quadtree(node, level=0):
  if node is not None:
    print(“ ” * level, f“[{node.x},{node.y}] {node.width}x{node.height}”)
    for child in node.children:
      display_quadtree(child, level + 4)
print(“\nQuadtree Structure:”)
display_quadtree(quadtree_root)
```

9. Caches and Buffers: While not explicitly part of the pseudo code, caches and buffers can be implemented to store frequently accessed data temporarily, reducing the need for repeated computations and improving overall performance.
10. Skip List: Skip lists could be used to manage the sub-goals for each prioritized problem, offering logarithmic time complexity for search, insertion, and deletion.

These performance improvements enable the system to efficiently survey user priorities, manage data, and generate personalized action items with gamified challenges for each sub-goal. By leveraging these data structures, the system can optimize memory usage, reduce redundant computations, and accelerate data retrieval, resulting in a faster, more responsive, and streamlined user experience.

Additionally, the system can leverage population benchmarking, longitudinal insights, and automated goal setting to provide personalized recommendations and interventions for users, improving health outcomes and promoting wellness.

In one embodiment that provides gamification with rewards and CBT. In this example, the code simulates a 1-week diabetes management program for a user. It generates random blood glucose levels and provides CBT feedback based on whether the blood glucose level is within the target range.

Points are awarded as gamification rewards based on the feedback received, and the method is as follows:

```
# Function to simulate blood glucose measurement
def measure_blood_glucose( ):
  return random.uniform(80, 180) # Simulated blood glucose level between 80 and 180 mg/dL
# Function to simulate CBT intervention for blood glucose control
def cbt_intervention(blood_glucose_level):
  target_range = (80, 130) # Target blood glucose range (mg/dL)
  if blood_glucose_level < target_range[0]:
    return “Great job! Your blood glucose is in the target range.”
  elif blood_glucose_level < target_range[1]:
    return “Keep it up! Your blood glucose is close to the target range.”
  else:
    return “Your blood glucose is high. Try to adjust your diet and medication.”
# Function to simulate gamification rewards and feedback
def gamification_rewards(cbt_feedback):
  points = 0
  if “Great job” in cbt_feedback:
    points += 5
  elif “Keep it up” in cbt_feedback:
    points += 2
  return points
# Main function simulating the diabetes management process
def main( ):
  user_name = input(“Enter your name: ”)
  print(f“Hello, {user_name}! Let's manage your diabetes together.”)
  total_points = 0
  for day in range(1, 8):
    print(f“Day {day}:”)
    blood_glucose = measure_blood_glucose( )
    cbt_feedback = cbt_intervention(blood_glucose)
    points = gamification_rewards(cbt_feedback)
    total_points += points
    print(cbt_feedback)
    print(f“Points earned today: {points}”)
    print(f“Total points: {total_points}\n”)
  print(“Congratulations on completing the week! Keep up the good work!”)
```

In one embodiment, the system applies generative AI as detailed in the co-pending applications, where the generative AI such as Bard, OpenAI, ChatGPT, or other large language models can be used to set or agree on a goal with the user based on the factor to be treated. The LLM is trained to provide a chatbot with the user and the LLM can be used to generate a responsive UI to interact and capture agreed upon goal as well as follow-up for the following goal setting actions:

| | | | |
|---|---|---|---|
| No. | Label | Definition | Examples |

1. Goals and planning

| | | | |
|---|---|---|---|
| 1.1 | Goal | Set or agree on a goal defined in terms | Agree on a daily walking goal |
| | setting | of the behavior to be achieved | (e.g. 3 miles) with the person |
| | (behavior) | Note: only code goal-setting if there is | and reach agreement about the |
| | | sufficient evidence that goal set as | goal |
| | | part of intervention; if goal | Set the goal of eating 5 pieces |
| | | unspecified or a behavioral outcome, | of fruit per day as specified in |
| | | code 1.3, Goal setting (outcome); if | public health guidelines |
| | | the goal defines a specific context, | |
| | | frequency, duration or intensity for the | |
| | | behavior, also code 1.4, Action | |
| | | planning | |
| 1.2 | Problem | Analyse , or prompt the person to | Identify specific triggers (e.g. |
| | solving | analyse, factors influencing the | being in a pub, feeling |
| | | behavior and generate or select | anxious) that generate the |
| | | strategies that include overcoming | urge/want/need to drink and |
| | | barriers and/or increasing facilitators | develop strategies for |
| | | (includes ‘Relapse Prevention’ and | avoiding environmental |
| | | ‘Coping Planning’) | triggers or for managing |
| | | Note: barrier identification without | negative emotions, such as |
| | | solutions is not sufficient. If the BCT | anxiety, that motivate |
| | | does not include analysing the | drinking |
| | | behavioral problem, consider 12.3, | Prompt the patient to identify |
| | | Avoidance/changing exposure to cues | barriers preventing them from |
| | | for the behavior, 12.1, Restructuring | starting a new exercise regime |
| | | the physical environment, 12.2, | e.g., lack of motivation, and |
| | | Restructuring the social | discuss ways in which they |
| | | environment, or 11.2, Reduce | could help overcome them |
| | | negative emotions | e.g., going to the gym with a |
| | | | buddy |
| 1.3 | Goal | Set or agree on a goal defined in terms | Set a weight loss goal (e.g. |
| | setting | of a positive outcome of wanted | 0.5 kilogram over one week) |
| | (outcome) | behavior | as an outcome of changed |
| | | Note: only code guidelines if set as a | eating patterns |
| | | goal in an intervention context; if goal | |
| | | is a behavior, code 1.1, Goal setting | |
| | | (behavior); if goal unspecified code | |
| | | 1.3, Goal setting (outcome) | |
| 1.4 | Action | Prompt detailed planning of | Encourage a plan to carry |
| | planning | performance of the behavior (must | condoms when going out |
| | | include at least one of context, | socially at weekends |
| | | frequency, duration and intensity). | Prompt planning the |
| | | Context may be environmental | performance of a particular |
| | | (physical or social) or internal | physical activity (e.g. |
| | | (physical, emotional or cognitive) | running) at a particular time |
| | | (includes ‘Implementation | (e.g. before work) on certain |
| | | Intentions') | days of the week |
| | | Note: evidence of action planning | |
| | | does not necessarily imply goal | |
| | | setting, only code latter if sufficient | |
| | | evidence | |
| 1.5 | Review | Review behavior goal(s) jointly with | Examine how well a person's |
| | behavior | the person and consider modifying | performance corresponds to |
| | goal(s) | goal(s) or behavior change strategy in | agreed goals e.g. whether they |
| | | light of achievement. This may lead to | consumed less than one unit |
| | | re-setting the same goal, a small | of alcohol per day, and |
| | | change in that goal or setting a new | consider modifying future |
| | | goal instead of (or in addition to) the | behavioral goals accordingly |
| | | first, or no change | e.g. by increasing or |
| | | Note: if goal specified in terms of | decreasing alcohol target or |
| | | behavior, code 1.5, Review behavior | changing type of alcohol |
| | | goal(s), if goal unspecified, code 1.7, | consumed |
| | | Review outcome goal(s); if | |
| | | discrepancy created consider also 1.6, | |
| | | Discrepancy between current | |
| | | behavior and goal | |
| 1.6 | Discrepancy | Draw attention to discrepancies | Point out that the recorded |
| | between | between a person's current behavior | exercise fell short of the goal |
| | current | (in terms of the form, frequency, | set |
| | behavior | duration, or intensity of that behavior) | |
| | and goal | and the person's previously set | |
| | | outcome goals, behavioral goals or | |
| | | action plans (goes beyond self- | |
| | | monitoring of behavior) | |
| | | Note: if discomfort is created only | |
| | | code 13.3, Incompatible beliefs and | |
| | | not 1.6, Discrepancy between current | |
| | | behavior and goal; if goals are | |
| | | modified, also code 1.5, Review | |
| | | behavior goal(s) and/or 1.7, Review | |
| | | outcome goal(s); if feedback is | |
| | | provided, also code 2.2, Feedback on | |
| | | behaviour | |
| 1.7 | Review | Review outcome goal(s) jointly with | Examine how much weight |
| | outcome | the person and consider modifying | has been lost and consider |
| | goal(s) | goal(s) in light of achievement. This | modifying outcome goal(s) |
| | | may lead to re-setting the same goal, a | accordingly e.g., by |
| | | small change in that goal or setting a | increasing or decreasing |
| | | new goal instead of, or in addition to | subsequent weight loss targets |
| | | the first | |
| | | Note: if goal specified in terms of | |
| | | behavior, code 1.5, Review behavior | |
| | | goal(s), if goal unspecified, code 1.7, | |
| | | Review outcome goal(s); if | |
| | | discrepancy created consider also 1.6, | |
| | | Discrepancy between current | |
| | | behavior and goal | |
| 1.8 | Behavioral | Create a written specification of the | Sign a contract with the |
| | contract | behavior to be performed, agreed on | person e.g. specifying that |
| | | by the person, and witnessed by | they will not drink alcohol for |
| | | another | one week |
| | | Note: also code 1.1, Goal setting | |
| | | (behavior) | |
| 1.9 | Commitment | Ask the person to affirm or reaffirm | Ask the person to use an “I |
| | | statements indicating commitment to | will” statement to affirm or |
| | | change the behavior | reaffirm a strong commitment |
| | | Note: if defined in terms of the | (i.e. using the words |
| | | behavior to be achieved also code 1.1, | “strongly”, “committed” or |
| | | Goal setting (behavior) | “high priority”) to start, |
| | | | continue or restart the attempt |
| | | | to take medication as |
| | | | prescribed |

2. Feedback and monitoring

| | | | |
|---|---|---|---|
| 2.1 | Monitoring | Observe or record behavior with the | Watch hand washing |
| | of behavior | person's knowledge as part of a | behaviors among health care |
| | by others | behavior change strategy | staff and make notes on |
| | without | Note: if monitoring is part of a data | context, frequency and |
| | feedback | collection procedure rather than a | technique used |
| | | strategy aimed at changing behavior, | |
| | | do not code; if feedback given, code | |
| | | only 2.2, Feedback on behavior, and | |
| | | not 2.1, Monitoring of behavior by | |
| | | others without feedback; if | |
| | | monitoring outcome(s) code 2.5, | |
| | | Monitoring outcome(s) of behavior | |
| | | by others without feedback; if self- | |
| | | monitoring behavior, code 2.3, Self- | |
| | | monitoring of behaviour | |
| 2.2 | Feedback | Monitor and provide informative or | Inform the person of how |
| | on | evaluative feedback on performance | many steps they walked each |
| | behavior | of the behavior (e.g. form, frequency, | day (as recorded on a |
| | | duration, intensity) | pedometer) or how many |
| | | Note: if Biofeedback, code only 2.6, | calories they ate each day |
| | | Biofeedback and not 2.2, Feedback | (based on a food consumption |
| | | on behavior; if feedback is on | questionnaire). |
| | | outcome(s) of behavior, code 2.7, | |
| | | Feedback on outcome(s) of behavior; | |
| | | if there is no clear evidence that | |
| | | feedback was given, code 2.1, | |
| | | Monitoring of behavior by others | |
| | | without feedback; if feedback on | |
| | | behaviour is evaluative e.g. praise, | |
| | | also code 10.4, Social reward | |
| 2.3 | Self- | Establish a method for the person to | Ask the person to record |
| | monitoring | monitor and record their behavior(s) | daily, in a diary, whether they |
| | of | as part of a behavior change strategy | have brushed their teeth for at |
| | behavior | Note: if monitoring is part of a data | least two minutes before |
| | | collection procedure rather than a | going to bed |
| | | strategy aimed at changing behavior, | Give patient a pedometer and |
| | | do not code; if monitoring of outcome | a form for recording daily |
| | | of behavior, code 2.4, Self-monitoring | total number of steps |
| | | of outcome(s) of behavior; if | |
| | | monitoring is by someone else | |
| | | (without feedback), code 2.1, | |
| | | Monitoring of behavior by others | |
| | | without feedback | |
| 2.4 | Self- | Establish a method for the person to | Ask the person to weigh |
| | monitoring | monitor and record the outcome(s) of | themselves at the end of each |
| | of | their behavior as part of a behavior | day, over a two week period, |
| | outcome(s) | change strategy | and record their daily weight |
| | of | Note: if monitoring is part of a data | on a graph to increase |
| | behavior | collection procedure rather than a | exercise behaviors |
| | | strategy aimed at changing behavior, | |
| | | do not code ; if monitoring behavior, | |
| | | code 2.3, Self-monitoring of | |
| | | behavior; if monitoring is by someone | |
| | | else (without feedback), code 2.5, | |
| | | Monitoring outcome(s) of behavior | |
| | | by others without feedback | |
| 2.5 | Monitoring | Observe or record outcomes of | Record blood pressure, blood |
| | outcome(s) | behavior with the person's knowledge | glucose, weight loss, or |
| | of | as part of a behavior change strategy | physical fitness |
| | behavior | Note: if monitoring is part of a data | |
| | by others | collection procedure rather than a | |
| | without | strategy aimed at changing behavior, | |
| | feedback | do not code; if feedback given, code | |
| | | only 2.7, Feedback on outcome(s) of | |
| | | behavior; if monitoring behavior code | |
| | | 2.1, Monitoring of behavior by others | |
| | | without feedback; if self-monitoring | |
| | | outcome(s), code 2.4, Self-monitoring | |
| | | of outcome(s) of behavior | |
| 2.6 | Biofeedback | Provide feedback about the body (e.g. | Inform the person of their |
| | | physiological or biochemical state) | blood pressure reading to |
| | | using an external monitoring device as | improve adoption of health |
| | | part of a behavior change strategy | behaviors |
| | | Note: if Biofeedback, code only 2.6, | |
| | | Biofeedback and not 2.2, Feedback | |
| | | on behavior or 2.7, Feedback on | |
| | | outcome(s) of behaviour | |
| 2.7 | Feedback | Monitor and provide feedback on the | Inform the person of how |
| | on | outcome of performance of the | much weight they have lost |
| | outcome(s) | behavior | following the implementation |
| | of | Note: if Biofeedback, code only 2.6, | of a new exercise regime |
| | behavior | Biofeedback and not 2.7, Feedback | |
| | | on outcome(s) of behavior; if | |
| | | feedback is on behavior code 2.2, | |
| | | Feedback on behavior; if there is no | |
| | | clear evidence that feedback was | |
| | | given code 2.5, Monitoring | |
| | | outcome(s) of behavior by others | |
| | | without feedback; if feedback on | |
| | | behaviour is evaluative e.g. praise, | |
| | | also code 10.4, Social reward | |

3. Social support

| | | | |
|---|---|---|---|
| 3.1 | Social | Advise on, arrange or provide social | Advise the person to call a |
| | support | support (e.g. from friends, relatives, | ‘buddy’ when they experience |
| | (unspecified) | colleagues, ‘buddies’ or staff) or non- | an urge to smoke |
| | | contingent praise or reward for | Arrange for a housemate to |
| | | performance of the behavior. It | encourage continuation with |
| | | includes encouragement and | the behavior change |
| | | counselling, but only when it is | programme |
| | | directed at the behavior | Give information about a self- |
| | | Note: attending a group class and/or | help group that offers support |
| | | mention of ‘follow-up’ does not | for the behavior |
| | | necessarily apply this BCT, support | |
| | | must be explicitly mentioned; if | |
| | | practical, code 3.2, Social support | |
| | | (practical); if emotional, code 3.3, | |
| | | Social support (emotional) (includes | |
| | | ‘Motivational interviewing’ and | |
| | | ‘Cognitive Behavioral Therapy’) | |
| 3.2 | Social | Advise on, arrange, or provide | Ask the partner of the patient |
| | support | practical help (e.g. from friends, | to put their tablet on the |
| | (practical) | relatives, colleagues, ‘buddies' or | breakfast tray so that the |
| | | staff) for performance of the behavior | patient remembers to take it |
| | | Note: if emotional, code 3.3, Social | |
| | | support (emotional); if general or | |
| | | unspecified, code 3.1, Social support | |
| | | (unspecified) If only restructuring the | |
| | | physical environment or adding | |
| | | objects to the environment, code 12.1, | |
| | | Restructuring the physical | |
| | | environment or 12.5, Adding objects | |
| | | to the environment; attending a group | |
| | | or class and/or mention of ‘follow-up’ | |
| | | does not necessarily apply this BCT, | |
| | | support must be explicitly mentioned. | |
| 3.3 | Social | Advise on, arrange, or provide | Ask the patient to take a |
| | support | emotional social support (e.g. from | partner or friend with them to |
| | (emotional) | friends, relatives, colleagues, | their colonoscopy |
| | | ‘buddies’ or staff) for performance of | appointment |
| | | the behavior | |
| | | Note: if practical, code 3.2, Social | |
| | | support (practical); if unspecified, | |
| | | code 3.1, Social support (unspecified) | |

4. Shaping knowledge

| | | | |
|---|---|---|---|
| 4.1 | Instruction | Advise or agree on how to perform the | Advise the person how to put |
| | on how to | behavior (includes ‘Skills training’) | a condom on a model of a |
| | perform a | Note: when the person attends classes | penis correctly |
| | behavior | such as exercise or cookery, code 4.1, | |
| | | Instruction on how to perform the | |
| | | behavior, 8.1, Behavioral | |
| | | practice/rehearsal and 6.1, | |
| | | Demonstration of the behavior | |
| 4.2 | Information | Provide information about antecedents | Advise to keep a record of |
| | about | (e.g. social and environmental | snacking and of situations or |
| | antecedents | situations and events, emotions, | events occurring prior to |
| | | cognitions) that reliably predict | snacking |
| | | performance of the behaviour | |
| 4.3 | Re- | Elicit perceived causes of behavior | If the person attributes their |
| | attribution | and suggest alternative explanations | over-eating to the frequent |
| | | (e.g. external or internal and stable or | presence of delicious food, |
| | | unstable) | suggest that the ‘real’ cause |
| | | | may be the person's |
| | | | inattention to bodily signals of |
| | | | hunger and satiety |
| 4.4 | Behavioral | Advise on how to identify and test | Ask a family physician to |
| | experiments | hypotheses about the behavior, its | give evidence-based advice |
| | | causes and consequences, by | rather than prescribe |
| | | collecting and interpreting data | antibiotics and to note |
| | | | whether the patients are |
| | | | grateful or annoyed |

5. Natural consequences

| | | | |
|---|---|---|---|
| 5.1 | Information | Provide information (e.g. written, | Explain that not finishing a |
| | about | verbal, visual) about health | course of antibiotics can |
| | health | consequences of performing the | increase susceptibility to |
| | consequences | behavior | future infection |
| | | Note: consequences can be for any | Present the likelihood of |
| | | target, not just the recipient(s) of the | contracting a sexually |
| | | intervention; emphasising importance | transmitted infection |
| | | of consequences is not sufficient; if | following unprotected sexual |
| | | information about emotional | behavior |
| | | consequences, code 5.6, Information | |
| | | about emotional consequences; if | |
| | | about social, environmental or | |
| | | unspecified consequences code 5.3, | |
| | | Information about social and | |
| | | environmental consequences | |
| 5.2 | Salience of | Use methods specifically designed to | Produce cigarette packets |
| | consequences | emphasise the consequences of | showing pictures of health |
| | | performing the behaviour with the aim | consequences e.g. diseased |
| | | of making them more memorable | lungs, to highlight the dangers |
| | | (goes beyond informing about | of continuing to smoke |
| | | consequences) | |
| | | Note: if information about | |
| | | consequences, also code 5.1, | |
| | | Information about health | |
| | | consequences, 5.6, Information about | |
| | | emotional consequences or 5.3, | |
| | | Information about social and | |
| | | environmental consequences | |
| 5.3 | Information | Provide information (e.g. written, | Tell family physician about |
| | about | verbal, visual) about social and | financial remuneration for |
| | social and | environmental consequences of | conducting health screening |
| | environmental | performing the behavior | Inform a smoker that the |
| | consequences | Note: consequences can be for any | majority of people disapprove |
| | | target, not just the recipient(s) of the | of smoking in public places |
| | | intervention; if information about | |
| | | health or consequences, code 5.1, | |
| | | Information about health | |
| | | consequences; if about emotional | |
| | | consequences, code 5.6, Information | |
| | | about emotional consequences; if | |
| | | unspecified, code 5.3, Information | |
| | | about social and environmental | |
| | | consequences | |
| 5.4 | Monitoring | Prompt assessment of feelings after | Agree that the person will |
| | of emotional | attempts at performing the behavior | record how they feel after |
| | consequences | | taking their daily walk |
| 5.5 | Anticipated | Induce or raise awareness of | Ask the person to assess the |
| | regret | expectations of future regret about | degree of regret they will feel |
| | | performance of the unwanted behavior | if they do not quit smoking |
| | | Note: not including 5.6, Information | |
| | | about emotional consequences; if | |
| | | suggests adoption of a perspective or | |
| | | new perspective in order to change | |
| | | cognitions also code 13.2, | |
| | | Framing/reframing | |
| 5.6 | Information | Provide information (e.g. written, | Explain that quitting smoking |
| | about | verbal, visual) about emotional | increases happiness and life |
| | emotional | consequences of performing the | satisfaction |
| | consequences | behavior | |
| | | Note: consequences can be related to | |
| | | emotional health disorders (e.g. | |
| | | depression, anxiety) and/or states of | |
| | | mind (e.g. low mood, stress); not | |
| | | including 5.5, Anticipated regret; | |
| | | consequences can be for any target, | |
| | | not just the recipient(s) of the | |
| | | intervention; if information about | |
| | | health consequences code 5.1, | |
| | | Information about health | |
| | | consequences; if about social, | |
| | | environmental or unspecified code 5.3, | |
| | | Information about social and | |
| | | environmental consequences | |

6. Comparison of behaviour

| | | | |
|---|---|---|---|
| 6.1 | Demonstration | Provide an observable sample of the | Demonstrate to nurses how to |
| | of the | performance of the behaviour, directly | raise the issue of excessive |
| | behavior | in person or indirectly e.g. via film, | drinking with patients via a |
| | | pictures, for the person to aspire to or | role-play exercise |
| | | imitate (includes ‘Modelling’). Note: | |
| | | if advised to practice, also code, 8.1, | |
| | | Behavioural practice and rehearsal; | |
| | | If provided with instructions on how to | |
| | | perform, also code 4.1, Instruction on | |
| | | how to perform the behaviour | |
| 6.2 | Social | Draw attention to others' performance | Show the doctor the |
| | comparison | to allow comparison with the person's | proportion of patients who |
| | | own performance Note: being in a | were prescribed antibiotics for |
| | | group setting does not necessarily | a common cold by other |
| | | mean that social comparison is | doctors and compare with |
| | | actually taking place | their own data |
| 6.3 | Information | Provide information about what other | Tell the staff at the hospital |
| | about | people think about the behavior. The | ward that staff at all other |
| | others' | information clarifies whether others | wards approve of washing |
| | approval | will like, approve or disapprove of | their hands according to the |
| | | what the person is doing or will do | guidelines |

7. Associations

| | | | |
|---|---|---|---|
| 7.1 | Prompts/ | Introduce or define environmental or | Put a sticker on the bathroom |
| | cues | social stimulus with the purpose of | mirror to remind people to |
| | | prompting or cueing the behavior. The | brush their teeth |
| | | prompt or cue would normally occur | |
| | | at the time or place of performance | |
| | | Note: when a stimulus is linked to a | |
| | | specific action in an if-then plan | |
| | | including one or more of frequency, | |
| | | duration or intensity also code 1.4, | |
| | | Action planning. | |
| 7.2 | Cue | Identify an environmental stimulus | Advise that a fee will be paid |
| | signalling | that reliably predicts that reward will | to dentists for a particular |
| | reward | follow the behavior (includes | dental treatment of 6-8 year |
| | | ‘Discriminative cue’) | old, but not older, children to |
| | | | encourage delivery of that |
| | | | treatment (the 6-8 year old |
| | | | children are the |
| | | | environmental stimulus) |
| 7.3 | Reduce | Withdraw gradually prompts to | Reduce gradually the number |
| | prompts/ | perform the behavior (includes | of reminders used to take |
| | cues | ‘Fading’) | medication |
| 7.4 | Remove | Advise or arrange for the person to be | Arrange for cupboard |
| | access to | separated from situations in which | containing high calorie snacks |
| | the | unwanted behavior can be rewarded in | to be locked for a specified |
| | reward | order to reduce the behavior (includes | period to reduce the |
| | | ‘Time out’) | consumption of sugary foods |
| | | | in between meals |
| 7.5 | Remove | Advise or arrange for the removal of | Arrange for a gym-buddy to |
| | aversive | an aversive stimulus to facilitate | stop nagging the person to do |
| | stimulus | behavior change (includes ‘Escape | more exercise in order to |
| | | learning’) | increase the desired exercise |
| | | | behaviour |
| 7.6 | Satiation | Advise or arrange repeated exposure | Arrange for the person to eat |
| | | to a stimulus that reduces or | large quantities of chocolate, |
| | | extinguishes a drive for the unwanted | in order to reduce the person's |
| | | behavior | appetite for sweet foods |
| 7.7 | Exposure | Provide systematic confrontation with | Agree a schedule by which |
| | | a feared stimulus to reduce the | the person who is frightened |
| | | response to a later encounter | of surgery will visit the |
| | | | hospital where they are |
| | | | scheduled to have surgery |
| 7.8 | Associative | Present a neutral stimulus jointly with | Present repeatedly fatty foods |
| | learning | a stimulus that already elicits the | with a disliked sauce to |
| | | behavior repeatedly until the neutral | discourage the consumption |
| | | stimulus elicits that behavior (includes | of fatty foods |
| | | ‘Classical/Pavlovian Conditioning’) | |
| | | Note: when a BCT involves reward or | |
| | | punishment, code one or more of: | |
| | | 10.2, Material reward (behavior); | |
| | | 10.3, Non-specific reward; 10.4, | |
| | | Social reward, 10.9, Self-reward; | |
| | | 10.10, Reward (outcome) | |

8. Repetition and substitution

| | | | |
|---|---|---|---|
| 8.1 | Behavioral | Prompt practice or rehearsal of the | Prompt asthma patients to |
| | practice/ | performance of the behavior one or | practice measuring their peak |
| | rehearsal | more times in a context or at a time | flow in the nurse's consulting |
| | | when the performance may not be | room |
| | | necessary, in order to increase habit | |
| | | and skill | |
| | | Note: if aiming to associate | |
| | | performance with the context, also | |
| | | code 8.3, Habit formation | |
| 8.2 | Behavior | Prompt substitution of the unwanted | Suggest that the person goes |
| | substitution | behavior with a wanted or neutral | for a walk rather than watches |
| | | behavior | television |
| | | Note: if this occurs regularly, also | |
| | | code 8.4, Habit reversal | |
| 8.3 | Habit | Prompt rehearsal and repetition of the | Prompt patients to take their |
| | formation | behavior in the same context | statin tablet before brushing |
| | | repeatedly so that the context elicits | their teeth every evening |
| | | the behavior | |
| | | Note: also code 8.1, Behavioral | |
| | | practice/rehearsal | |
| 8.4 | Habit | Prompt rehearsal and repetition of an | Ask the person to walk up |
| | reversal | alternative behavior to replace an | stairs at work where they |
| | | unwanted habitual behavior | previously always took the lift |
| | | Note: also code 8.2, Behavior | |
| | | substitution | |
| 8.5 | Overcorrection | Ask to repeat the wanted behavior in | Ask to eat only fruit and |
| | | an exaggerated way following an | vegetables the day after a |
| | | unwanted behaviour | poor diet |
| 8.6 | Generalisation | Advise to perform the wanted | Advise to repeat toning |
| | of a target | behaviour, which is already | exercises learned in the gym |
| | behavior | performed in a particular situation, in | when at home |
| | | another situation | |
| 8.7 | Graded | Set easy-to-perform tasks, making | Ask the person to walk for |
| | tasks | them increasingly difficult, but | 100 yards a day for the first |
| | | achievable, until behavior is | week, then half a mile a day |
| | | performed | after they have successfully |
| | | | achieved 100 yards, then two |
| | | | miles a day after they have |
| | | | successfully achieved one |
| | | | mile |

9. Comparison of outcomes

| | | | |
|---|---|---|---|
| 9.1 | Credible | Present verbal or visual | Present a speech given by a |
| | source | communication from a credible source | high status professional to |
| | | in favour of or against the behavior | emphasise the importance of |
| | | Note: code this BCT if source | not exposing patients to |
| | | generally agreed on as credible e.g., | unnecessary radiation by |
| | | health professionals, celebrities or | ordering x-rays for back pain |
| | | words used to indicate expertise or | |
| | | leader in field and if the | |
| | | communication has the aim of | |
| | | persuading; if information about | |
| | | health consequences, also code 5.1, | |
| | | Information about health | |
| | | consequences, if about emotional | |
| | | consequences, also code 5.6, | |
| | | Information about emotional | |
| | | consequences; if about social, | |
| | | environmental or unspecified | |
| | | consequences also code 5.3, | |
| | | Information about social and | |
| | | environmental consequences | |
| 9.2 | Pros and | Advise the person to identify and | Advise the person to list and |
| | cons | compare reasons for wanting (pros) | compare the advantages and |
| | | and not wanting to (cons) change the | disadvantages of prescribing |
| | | behavior (includes ‘Decisional | antibiotics for upper |
| | | balance’ | respiratory tract infections |
| | | Note: if providing information about | |
| | | health consequences, also code 5.1, | |
| | | Information about health | |
| | | consequences; if providing | |
| | | information about emotional | |
| | | consequences, also code 5.6, | |
| | | Information about emotional | |
| | | consequences; if providing | |
| | | information about social, | |
| | | environmental or unspecified | |
| | | consequences also code 5.3, | |
| | | Information about social and | |
| | | environmental consequences | |
| 9.3 | Comparative | Prompt or advise the imagining and | Prompt the person to imagine |
| | imagining | comparing of future outcomes of | and compare likely or |
| | of future | changed versus unchanged behaviour | possible outcomes following |
| | outcomes | | attending versus not attending |
| | | | a screening appointment |

10. Reward and threat

| | | | |
|---|---|---|---|
| 10.1 | Material | Inform that money, vouchers or other | Inform that a financial |
| | incentive | valued objects will be delivered if and | payment will be made each |
| | (behavior) | only if there has been effort and/or | month in pregnancy that the |
| | | progress in performing the behavior | woman has not smoked |
| | | (includes ‘Positive reinforcement’) | |
| | | Note: if incentive is social, code 10.5, | |
| | | Social incentive if unspecified code | |
| | | 10.6, Non-specific incentive, and not | |
| | | 10.1, Material incentive (behavior); if | |
| | | incentive is for outcome, code 10.8, | |
| | | Incentive (outcome). If reward is | |
| | | delivered also code one of: 10.2, | |
| | | Material reward (behavior); 10.3, | |
| | | Non-specific reward; 10.4, Social | |
| | | reward, 10.9, Self-reward; 10.10, | |
| | | Reward (outcome) | |
| 10.2 | Material | Arrange for the delivery of money, | Arrange for the person to |
| | reward | vouchers or other valued objects if and | receive money that would |
| | (behavior) | only if there has been effort and/or | have been spent on cigarettes |
| | | progress in performing the behavior | if and only if the smoker has |
| | | (includes ‘Positive reinforcement’) | not smoked for one month |
| | | Note: If reward is social, code 10.4, | |
| | | Social reward, if unspecified code | |
| | | 10.3, Non-specific reward, and not | |
| | | 10.1, Material reward (behavior); if | |
| | | reward is for outcome, code 10.10, | |
| | | Reward (outcome). If informed of | |
| | | reward in advance of rewarded | |
| | | behaviour, also code one of: 10.1, | |
| | | Material incentive (behaviour); 10.5, | |
| | | Social incentive; 10.6, Non-specific | |
| | | incentive; 10.7, Self-incentive; 10.8, | |
| | | Incentive (outcome) | |
| 10.3 | Non- | Arrange delivery of a reward if and | Identify something (e.g. an |
| | specific | only if there has been effort and/or | activity such as a visit to the |
| | reward | progress in performing the behavior | cinema) that the person values |
| | | (includes ‘Positive reinforcement’) | and arrange for this to be |
| | | Note: if reward is material, code 10.2, | delivered if and only if they |
| | | Material reward (behavior), if social, | attend for health screening |
| | | code 10.4, Social reward, and not | |
| | | 10.3, Non-specific reward; if reward | |
| | | is for outcome code 10.10, Reward | |
| | | (outcome). If informed of reward in | |
| | | advance of rewarded behaviour, also | |
| | | code one of: 10.1, Material incentive | |
| | | (behaviour); 10.5, Social incentive; | |
| | | 10.6, Non-specific incentive; 10.7, | |
| | | Self-incentive; 10.8, Incentive | |
| | | (outcome) | |
| 10.4 | Social | Arrange verbal or non-verbal reward if | Congratulate the person for |
| | reward | and only if there has been effort | each day they eat a reduced |
| | | and/or progress in performing the | fat diet |
| | | behavior (includes ‘Positive | |
| | | reinforcement’) | |
| | | Note: if reward is material, code 10.2, | |
| | | Material reward (behavior), if | |
| | | unspecified code 10.3, Non-specific | |
| | | reward, and not 10.4, Social reward; | |
| | | if reward is for outcome code 10.10, | |
| | | Reward (outcome). If informed of | |
| | | reward in advance of rewarded | |
| | | behaviour, also code one of: 10.1, | |
| | | Material incentive (behaviour); 10.5, | |
| | | Social incentive; 10.6, Non-specific | |
| | | incentive; 10.7, Self-incentive; 10.8, | |
| | | Incentive (outcome) | |
| 10.5 | Social | Inform that a verbal or non-verbal | Inform that they will be |
| | incentive | reward will be delivered if and only if | congratulated for each day |
| | | there has been effort and/or progress | they eat a reduced fat diet |
| | | in performing the behavior (includes | |
| | | ‘Positive reinforcement’) | |
| | | Note: if incentive is material, code | |
| | | 10.1, Material incentive (behavior), if | |
| | | unspecified code 10.6, Non-specific | |
| | | incentive, and not 10.5, Social | |
| | | incentive; if incentive is for outcome | |
| | | code 10.8, Incentive (outcome). If | |
| | | reward is delivered also code one of: | |
| | | 10.2, Material reward (behavior); | |
| | | 10.3, Non-specific reward; 10.4, | |
| | | Social reward, 10.9, Self-reward; | |
| | | 10.10, Reward (outcome) | |
| 10.6 | Non- | Inform that a reward will be delivered | Identify an activity that the |
| | specific | if and only if there has been effort | person values and inform |
| | incentive | and/or progress in performing the | them that this will happen if |
| | | behavior (includes ‘Positive | and only if they attend for |
| | | reinforcement’) | health screening |
| | | Note: if incentive is material, code | |
| | | 10.1, Material incentive (behavior), if | |
| | | social, code 10.5, Social incentive and | |
| | | not 10.6, Non-specific incentive; if | |
| | | incentive is for outcome code 10.8, | |
| | | Incentive (outcome). If reward is | |
| | | delivered also code one of: 10.2, | |
| | | Material reward (behavior); 10.3, | |
| | | Non-specific reward; 10.4, Social | |
| | | reward, 10.9, Self-reward; 10.10, | |
| | | Reward (outcome) | |
| 10.7 | Self- | Plan to reward self in future if and | Encourage to provide self |
| | incentive | only if there has been effort and/or | with material (e.g., new |
| | | progress in performing the behavior | clothes) or other valued |
| | | Note: if self-reward is material, also | objects if and only if they |
| | | code 10.1, Material incentive | have adhered to a healthy diet |
| | | (behavior), if social, also code 10.5, | |
| | | Social incentive, if unspecified, also | |
| | | code 10.6, Non-specific incentive; if | |
| | | incentive is for outcome code 10.8, | |
| | | Incentive (outcome). If reward is | |
| | | delivered also code one of: 10.2, | |
| | | Material reward (behavior); 10.3, | |
| | | Non-specific reward; 10.4, Social | |
| | | reward, 10.9, Self-reward; 10.10, | |
| | | Reward (outcome) | |
| 10.8 | Incentive | Inform that a reward will be delivered | Inform the person that they |
| | (outcome) | if and only if there has been effort | will receive money if and |
| | | and/or progress in achieving the | only if a certain amount of |
| | | behavioural outcome (includes | weight is lost |
| | | ‘Positive reinforcement’) | |
| | | Note: this includes social, material, | |
| | | self- and non-specific incentives for | |
| | | outcome; if incentive is for the | |
| | | behavior code 10.5, Social incentive, | |
| | | 10.1, Material incentive (behavior), | |
| | | 10.6, Non-specific incentive or 10.7, | |
| | | Self-incentive and not 10.8, Incentive | |
| | | (outcome). If reward is delivered also | |
| | | code one of: 10.2, Material reward | |
| | | (behavior); 10.3, Non-specific | |
| | | reward; 10.4, Social reward, 10.9, | |
| | | Self-reward; 10.10, Reward | |
| | | (outcome) | |
| 10.9 | Self- | Prompt self-praise or self-reward if | Encourage to reward self with |
| | reward | and only if there has been effort | material (e.g., new clothes) or |
| | | and/or progress in performing the | other valued objects if and |
| | | behavior | only if they have adhered to a |
| | | Note: if self-reward is material, also | healthy diet |
| | | code 10.2, Material reward | |
| | | (behavior), if social, also code 10.4, | |
| | | Social reward, if unspecified, also | |
| | | code 10.3, Non-specific reward; if | |
| | | reward is for outcome code 10.10, | |
| | | Reward (outcome). If informed of | |
| | | reward in advance of rewarded | |
| | | behaviour, also code one of: 10.1, | |
| | | Material incentive (behaviour); 10.5, | |
| | | Social incentive; 10.6, Non-specific | |
| | | incentive; 10.7, Self-incentive; 10.8, | |
| | | Incentive (outcome) | |
| 10.10 | Reward | Arrange for the delivery of a reward if | Arrange for the person to |
| | (outcome) | and only if there has been effort | receive money if and only if a |
| | | and/or progress in achieving the | certain amount of weight is |
| | | behavioral outcome (includes | lost |
| | | ‘Positive reinforcement’) | |
| | | Note: this includes social, material, | |
| | | self- and non-specific rewards for | |
| | | outcome; if reward is for the behavior | |
| | | code 10.4, Social reward, 10.2, | |
| | | Material reward (behavior), 10.3, | |
| | | Non-specific reward or 10.9, Self- | |
| | | reward and not 10.10, Reward | |
| | | (outcome). If informed of reward in | |
| | | advance of rewarded behaviour, also | |
| | | code one of: 10.1, Material incentive | |
| | | (behaviour); 10.5, Social incentive; | |
| | | 10.6, Non-specific incentive; 10.7, | |
| | | Self-incentive; 10.8, Incentive | |
| | | (outcome) | |
| 10.11 | Future | Inform that future punishment or | Inform that continuing to |
| | punishment | removal of reward will be a | consume 30 units of alcohol |
| | | consequence of performance of an | per day is likely to result in |
| | | unwanted behavior (may include fear | loss of employment if the |
| | | arousal) (includes ‘Threat’) | person continues |

11. Regulation

| | | | |
|---|---|---|---|
| 11.1 | Pharma | Provide, or encourage the use of or | Suggest the patient asks the |
| | cological | adherence to, drugs to facilitate | family physician for nicotine |
| | support | behavior change | replacement therapy to |
| | | Note: if pharmacological support to | facilitate smoking cessation |
| | | reduce negative emotions (i.e. anxiety) | |
| | | then also code 11.2, Reduce negative | |
| | | emotions | |
| 11.2 | Reduce | Advise on ways of reducing negative | Advise on the use of stress |
| | negative | emotions to facilitate performance of | management skills, e.g. to |
| | emotions^{b} | the behavior (includes ‘Stress | reduce anxiety about joining |
| | | Management’) | Alcoholics Anonymous |
| | | Note: if includes analysing the | |
| | | behavioural problem, also code 1.2, | |
| | | Problem solving | |
| 11.3 | Conserving | Advise on ways of minimising | Advise to carry food calorie |
| | mental | demands on mental resources to | content information to reduce |
| | resources | facilitate behavior change | the burden on memory in |
| | | | making food choices |
| 11.4 | Paradoxical | Advise to engage in some form of the | Advise a smoker to smoke |
| | instructions | unwanted behavior with the aim of | twice as many cigarettes a day |
| | | reducing motivation to engage in that | as they usually do |
| | | behaviour | Tell the person to stay awake |
| | | | as long as possible in order to |
| | | | reduce insomnia |

12. Antecedents

| | | | |
|---|---|---|---|
| 12.1 | Restructuring | Change, or advise to change the | Advise to keep biscuits and |
| | the physical | physical environment in order to | snacks in a cupboard that is |
| | environment | facilitate performance of the wanted | inconvenient to get to |
| | | behavior or create barriers to the | Arrange to move vending |
| | | unwanted behavior (other than | machine out of the school |
| | | prompts/cues, rewards and | |
| | | punishments) | |
| | | Note: this may also involve 12.3, | |
| | | Avoidance/reducing exposure to cues | |
| | | for the behavior; if restructuring of | |
| | | the social environment code 12.2, | |
| | | Restructuring the social | |
| | | environment; | |
| | | if only adding objects to the | |
| | | environment, code 12.5, Adding | |
| | | objects to the environment | |
| 12.2 | Restructuring | Change, or advise to change the social | Advise to minimise time spent |
| | the social | environment in order to facilitate | with friends who drink |
| | environment | performance of the wanted behavior or | heavily to reduce alcohol |
| | | create barriers to the unwanted | consumption |
| | | behavior (other than prompts/cues, | |
| | | rewards and punishments) | |
| | | Note: this may also involve 12.3, | |
| | | Avoidance/reducing exposure to cues | |
| | | for the behavior; if also restructuring | |
| | | of the physical environment also code | |
| | | 12.1, Restructuring the physical | |
| | | environment | |
| 12.3 | Avoidance/ | Advise on how to avoid exposure to | Suggest to a person who |
| | reducing | specific social and contextual/physical | wants to quit smoking that |
| | exposure | cues for the behavior, including | their social life focus on |
| | to cues | changing daily or weekly routines | activities other than pubs and |
| | for the | Note: this may also involve 12.1, | bars which have been |
| | behavior | Restructuring the physical | associated with smoking |
| | | environment and/or 12.2, | |
| | | Restructuring the social | |
| | | environment; if the BCT includes | |
| | | analysing the behavioral problem, | |
| | | only code 1.2, Problem solving | |
| 12.4 | Distraction | Advise or arrange to use an alternative | Suggest to a person who is |
| | | focus for attention to avoid triggers for | trying to avoid between-meal |
| | | unwanted behaviour | snacking to focus on a topic |
| | | | they enjoy (e.g. holiday plans) |
| | | | instead of focusing on food |
| 12.5 | Adding | Add objects to the environment in | Provide free condoms to |
| | objects | order to facilitate performance of the | facilitate safe sex |
| | to the | behavior | Provide attractive toothbrush |
| | environment | Note: Provision of information (e.g. | to improve tooth brushing |
| | | written, verbal, visual) in a booklet or | technique |
| | | leaflet is insufficient. If this is | |
| | | accompanied by social support, also | |
| | | code 3.2, Social support (practical); if | |
| | | the environment is changed beyond the | |
| | | addition of objects, also code 12.1, | |
| | | Restructuring the physical | |
| | | environment | |
| 12.6 | Body | Alter body structure, functioning or | Prompt strength training, |
| | changes | support directly to facilitate behavior | relaxation training or provide |
| | | change | assistive aids (e.g. a hearing |
| | | | aid) |

13. Identity

| | | | |
|---|---|---|---|
| 13.1 | Identification | Inform that one's own behavior may | Inform the person that if they |
| | of self as | be an example to others | eat healthily, that may be a |
| | role model | | good example for their |
| | | | children |
| 13.2 | Framing/ | Suggest the deliberate adoption of a | Suggest that the person might |
| | reframing | perspective or new perspective on | think of the tasks as reducing |
| | | behavior (e.g. its purpose) in order to | sedentary behavior (rather |
| | | change cognitions or emotions about | than increasing activity) |
| | | performing the behavior (includes | |
| | | ‘Cognitive structuring’); If | |
| | | information about consequences then | |
| | | code 5.1, Information about health | |
| | | consequences, 5.6, Information about | |
| | | emotional consequences or 5.3, | |
| | | Information about social and | |
| | | environmental consequences instead | |
| | | of 13.2, Framing/reframing | |
| 13.3 | Incompatible | Draw attention to discrepancies | Draw attention to a doctor's |
| | beliefs | between current or past behavior and | liberal use of blood |
| | | self-image, in order to create | transfusion and their self- |
| | | discomfort (includes ‘Cognitive | identification as a proponent |
| | | dissonance’) | of evidence-based medical |
| | | | practice |
| 13.4 | Valued | Advise the person to write or complete | Advise the person to write |
| | self- | rating scales about a cherished value | about their personal strengths |
| | identity | or personal strength as a means of | before they receive a message |
| | | affirming the person's identity as part | advocating the behavior |
| | | of a behavior change strategy | change |
| | | (includes ‘Self-affirmation’) | |
| 13.5 | Identity | Advise the person to construct a new | Ask the person to articulate |
| | associated | self-identity as someone who ‘used to | their new identity as an ‘ex- |
| | with | engage with the unwanted behavior’ | smoker’ |
| | changed | | |
| | behavior | | |

14. Scheduled consequences

| | | | |
|---|---|---|---|
| 14.1 | Behavior | Arrange for withdrawal of something | Subtract money from a |
| | cost | valued if and only if an unwanted | prepaid refundable deposit |
| | | behavior is performed (includes | when a cigarette is smoked |
| | | ‘Response cost’). Note if withdrawal | |
| | | of contingent reward code, 14.3, | |
| | | Remove reward | |
| 14.2 | Punishment | Arrange for aversive consequence | Arrange for the person to |
| | | contingent on the performance of the | wear unattractive clothes |
| | | unwanted behavior | following consumption of |
| | | | fatty foods |
| 14.3 | Remove | Arrange for discontinuation of | Arrange for the other people |
| | reward | contingent reward following | in the household to ignore the |
| | | performance of the unwanted behavior | person every time they eat |
| | | (includes ‘Extinction’) | chocolate (rather than |
| | | | attending to them by |
| | | | criticising or persuading) |
| 14.4 | Reward | Arrange for reward following any | Arrange reward for any |
| | approximation | approximation to the target behavior, | reduction in daily calories, |
| | | gradually rewarding only performance | gradually requiring the daily |
| | | closer to the wanted behavior | calorie count to become closer |
| | | (includes ‘Shaping’) | to the planned calorie intake |
| | | Note: also code one of 59-63 | |
| 14.5 | Rewarding | Build up behavior by arranging reward | Reward eating a supplied low |
| | completion | following final component of the | calorie meal; then make |
| | | behavior; gradually add the | reward contingent on cooking |
| | | components of the behavior that occur | and eating the meal; then |
| | | earlier in the behavioral sequence | make reward contingent on |
| | | (includes ‘Backward chaining’) | purchasing, cooking and |
| | | Note: also code one of 10.2, Material | eating the meal |
| | | reward (behavior); 10.3, Non-specific | |
| | | reward; 10.4, Social reward, 10.9, | |
| | | Self-reward; 10.10, Reward | |
| | | (outcome) | |
| 14.6 | Situation- | Arrange for reward following the | Arrange reward for eating at |
| | specific | behavior in one situation but not in | mealtimes but not between |
| | reward | another (includes ‘Discrimination | meals |
| | | training’ | |
| | | Note: also code one of 10.2, Material | |
| | | reward (behavior); 10.3, Non-specific | |
| | | reward; 10.4, Social reward, 10.9, | |
| | | Self-reward; 10.10, Reward | |
| | | (outcome) | |
| 14.7 | Reward | Arrange reward for responding in a | Arrange reward for ordering |
| | incompatible | manner that is incompatible with a | a soft drink at the bar rather |
| | behavior | previous response to that situation | than an alcoholic beverage |
| | | (includes ‘Counter-conditioning’) | |
| | | Note: also code one of 10.2, Material | |
| | | reward (behavior); 10.3, Non-specific | |
| | | reward; 10.4, Social reward, 10.9, | |
| | | Self-reward; 10.10, Reward | |
| | | (outcome) | |
| 14.8 | Reward | Arrange reward for performance of an | Reward for consumption of |
| | alternative | alternative to the unwanted behavior | low fat foods but not |
| | behavior | (includes ‘Differential | consumption of high fat foods |
| | | reinforcement’) | |
| | | Note: also code one of 10.2, Material | |
| | | reward (behavior); 10.3, Non-specific | |
| | | reward; 10.4, Social reward, 10.9, | |
| | | Self-reward; 10.10, Reward | |
| | | (outcome); consider also coding 1.2, | |
| | | Problem solving | |
| 14.9 | Reduce | Arrange for rewards to be made | Arrange reward for each day |
| | reward | contingent on increasing duration or | without smoking, then each |
| | frequency | frequency of the behavior (includes | week, then each month, then |
| | | ‘Thinning’) | every 2 months and so on |
| | | Note: also code one of 10.2, Material | |
| | | reward (behavior); 10.3, Non-specific | |
| | | reward; 10.4, Social reward, 10.9, | |
| | | Self-reward; 10.10, Reward | |
| | | (outcome) | |
| 14.10 | Remove | Arrange for removal of an unpleasant | Arrange for someone else to |
| | punishment | consequence contingent on | do housecleaning only if the |
| | | performance of the wanted behavior | person has adhered to the |
| | | (includes ‘Negative reinforcement’) | medication regimen for a |
| | | | week |

15. Self-belief

| | | | |
|---|---|---|---|
| 15.1 | Verbal | Tell the person that they can | Tell the person that they can |
| | persuasion | successfully perform the wanted | successfully increase their |
| | about | behavior, arguing against self-doubts | physical activity, despite their |
| | capability | and asserting that they can and will | recent heart attack. |
| | | succeed | |
| 15.2 | Mental | Advise to practise imagining | Advise to imagine eating and |
| | rehearsal of | performing the behavior successfully | enjoying a salad in a work |
| | successful | in relevant contexts | canteen |
| | performance | | |
| 15.3 | Focus | Advise to think about or list previous | Advise to describe or list the |
| | on past | successes in performing the behavior | occasions on which the person |
| | success | (or parts of it) | had ordered a non-alcoholic |
| | | | drink in a bar |
| 15.4 | Self-talk | Prompt positive self-talk (aloud or | Prompt the person to tell |
| | | silently) before and during the | themselves that a walk will be |
| | | behavior | energising |

16. Covert learning

| | | | |
|---|---|---|---|
| 16.1 | Imaginary | Advise to imagine performing the | Advise to imagine overeating |
| | punishment | unwanted behavior in a real-life | and then vomiting |
| | | situation followed by imagining an | |
| | | unpleasant consequence (includes | |
| | | ‘Covert sensitisation’) | |
| 16.2 | Imaginary | Advise to imagine performing the | Advise the health professional |
| | reward | wanted behavior in a real-life | to imagine giving dietary |
| | | situation followed by imagining a | advice followed by the patient |
| | | pleasant consequence (includes | losing weight and no longer |
| | | ‘Covert conditioning’) | being diabetic |
| 16.3 | Vicarious | Prompt observation of the | Draw attention to the positive |
| | consequences | consequences (including rewards and | comments other staff get |
| | | punishments) for others when they | when they disinfect their |
| | | perform the behavior | hands regularly |
| | | Note: if observation of health | |
| | | consequences, also code 5.1, | |
| | | Information about health | |
| | | consequences; if of emotional | |
| | | consequences, also code 5.6, | |
| | | Information about emotional | |
| | | consequences, if of social, | |
| | | environmental or unspecified | |
| | | consequences, also code 5.3, | |
| | | Information about social and | |
| | | environmental consequences | |

While the techniques discussed herein are well-suited to serving diabetic or hypertension patients, the same techniques can also be applied to provide digital therapeutics and improve wellness in other people also. For example, people who have a chronic physical condition, such as arthritis, diabetes, hepatitis, heart disease, COPD, etc., can also benefit from the application of various digital therapeutics programs and the analysis and adjustment in programs that the system provides. Similarly, the system may be used to treat and support users with psychological conditions such as depression, anxiety disorder, attention-deficit/hyperactivity disorder, bipolar disorder, etc. To assists these users, and any of the other types of users, the system may use cognitive behavioral therapy techniques to assist the users in adjusting behaviors, mood, etc.

As another example, the system can be used to treat and support users recovering from a discrete event, such as surgery or trauma, e.g., a broken bone, a joint replacement, cataract surgery, and so on. As another example, the system may be used to support users in achieving general health, e.g., physical and/or psychological wellbeing. The system can encourage and guide users to improve or maintain a lifestyle on a day-to-day, or even moment-to-moment basis, as the various digital therapeutics programs determine are appropriate for the user given the current situation. As an example, programs for nutrition and physical fitness can prompt users to take incremental steps to reach goals that are set by the user or by the system. For all of these potential uses of the system and the different types of users, the ability for various digital therapeutics programs to interact and dynamically set their can provide improved responsiveness and efficiency of the system. Accordingly, in some implementations, the system can be used with different types of users who are not cancer patients or cancer survivors.

As discussed above, the states of different programs can be dynamically adjusted, based on current information about a user, historical information about the user, and based on the states of other programs. In addition, the types of interconnections between programs, e.g., the rules that define transitions between program states can also dynamically updated based on various factors. For example, the system can use information about the progress and symptoms of users over time can be used to identify conditions or triggers that should cause state transitions. Groups of users that have certain commonalities can be identified and their progress assessed to determine these conditions and triggers, and which actions to perform, e.g., which programs to activate or deactivate, and which levels are most effective. In addition to or instead of using data about users of the system, population-level data can be used in a similar manner to determine which combinations of programs and interventions are appropriate for different users. The population-level data may represent information about a population of a city, county, state or province, country, continent, or the world. Combining information from the data sets of users of the system with population-level data can provide increased accuracy of predictions, better enabling the system to identify predicted interactions and interventions that will address the patient's current or expected needs.

Embodiments of the invention and all of the functional operations described in this specification may be implemented in digital electronic circuitry, or in computer software, firmware, or hardware, including the structures disclosed in this specification and their structural equivalents, or in combinations of one or more of them. Embodiments of the invention may be implemented as one or more computer program products, i.e., one or more modules of computer program instructions encoded on a computer-readable medium for execution by, or to control the operation of, data processing apparatus. The computer readable medium may be a non-transitory computer readable storage medium, a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a stand-alone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, embodiments of the invention may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

Embodiments of the invention may be implemented in a computing system that includes a back end component, e.g., as a data server, or that includes a middleware component, e.g., an application server, or that includes a front end component, e.g., a client computer having a graphical user interface or a Web browser through which a user may interact with an implementation of the invention, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Thus, particular embodiments of the invention have been described. Other embodiments are within the scope of the following claims. For example, the actions recited in the claims may be performed in a different order and still achieve desirable results.

What is claimed is:

1. A computer-implemented method for health management, comprising:
determining one or more factors capable of altering a medical condition of a user;
prioritizing the one or more factors using behavioral coaching techniques;
generating sub-goal recommendations for each prioritized factor;
for each prioritized factor, determining one or more personalized sub-goals dynamically adjusted based on the user's health trajectory;
periodically collecting health or fitness data from a wearable device, comprising at least one of blood pressure data, HRV data, SpO2 data, skin temperature data, or physical activity data;
analyzing the collected data using machine learning (ML) to establish a personalized physiological baseline, detect deviations, and identify individual behavior patterns and sub-goal progress; and
iteratively generating personalized behavioral interventions delivered via voice coaching, visual alerts, or goal adjustment notifications, adapted in real time.

2. The method of claim 1, wherein the wearable device is a smart watch or an ear device, and wherein the health or fitness data comprises heart rate variability data, blood oxygen saturation data, and skin temperature data collected continuously during sleep.

3. The method of claim 1, wherein the medical condition comprises at least one of diabetes, hypertension, or cardiovascular disease, and wherein the factors comprise at least two of genetics, obesity, physical inactivity, diet, stress, age, or smoking.

4. The method of claim 1, wherein the personalized behavioral interventions are delivered via voice coaching through a wireless audio device connected to the wearable device, and wherein the voice coaching is generated by a large language model (LLM) that processes real-time biometric data from the wearable device.

5. The method of claim 1, further comprising gamifying the one or more sub-goals by: awarding points for completion of daily sub-goals; awarding badges or trophies for reaching predetermined health milestones; implementing health streaks that reward consecutive days of sub-goal completion; and displaying a leaderboard comparing the user's progress against other users.

6. The method of claim 1, further comprising: clustering the user with a population of users having similar health profiles; calculating average health metrics and identifying top-performer health outcomes within the cluster; and generating motivational feedback that compares the user's health metrics and sub-goal progress against the cluster average and top-performer metrics.

7. The method of claim 1, further comprising: receiving food or drink intake data from the user; predicting, using machine learning, a blood glucose level of the user over a predetermined time period based on the food or drink intake data; and generating a behavioral intervention to maintain the predicted blood glucose level within a medically safe range.

8. The method of claim 1, further comprising: determining a stress level of the user from wearable device data; predicting, using machine learning, a physiological response to the determined stress level over a predetermined time period; and generating a mindfulness or relaxation behavioral intervention when the predicted physiological response exceeds a threshold.

9. The method of claim 1, wherein the ML comprise reinforcement learning that recalibrates the one or more sub-goals over a rolling time window based on the user's accumulated performance data, recovery metrics, and behavioral consistency.

10. The method of claim 1, further comprising: collecting, over a longitudinal period, health data comprising at least medical records and wearable device biosensor data; applying machine learning algorithms to the longitudinal health data to identify long-term trends and patterns in health metrics; and adjusting the one or more sub-goals and personalized behavioral interventions based on the identified long-term trends.

11. The method of claim 1, further comprising: maintaining a medication database storing information about pharmaceuticals, medication effects, and relationships between medication effects and behavioral coaching programs; and adjusting the one or more sub-goals and behavioral coaching program based on the user's current medications and medication history.

12. The method of claim 1, wherein generating the personalized behavioral interventions comprises: generating, using a large language model (LLM), personalized text messages, reminders, or educational content related to the user's health condition and sub-goals; and adapting the generated content in real time based on the user's current biometric data from the wearable device.

13. The method of claim 1, further comprising: monitoring a sleep condition of the user via the wearable device; predicting a physiological health outcome based on the monitored sleep condition; and generating a behavioral intervention to improve the sleep condition and the predicted physiological health outcome.

14. The method of claim 1, wherein the wearable device comprises at least one of a smartwatch, a smart ring, or an ear-worn device, and wherein the health or fitness data is streamed in real time from the wearable device to a processor executing the ML algorithms.

15. The method of claim 1, further comprising: presenting the user with a survey identifying a plurality of health problems to prioritize; receiving from the user a selection of one or more prioritized health problems from the plurality of health problems; and for each prioritized health problem, generating a plurality of weekly sub-goals and gamified action items.

16. The method of claim 5, further comprising: comparing the user's health performance against a population of similar users; generating a motivational notification when the user's performance on one or more sub-goals surpasses a cluster average; and adjusting the leaderboard ranking in real time based on updated wearable device data.

17. The method of claim 1, further comprising: detecting a deviation of at least one health metric from the user's personalized physiological baseline; generating an alert notification to the user identifying the detected deviation; and recommending a specific behavioral intervention to address the detected deviation.

18. The method of claim 1, wherein the behavioral coaching techniques comprise at least one of: habit formation prompting the user to repeatedly perform a desired behavior in the same context; behavior substitution prompting replacement of an unwanted behavior with a wanted behavior; graded task assignment setting progressively more difficult but achievable sub-goals; or self-monitoring providing the user with tools to record and track their own health behaviors.

19. The method of claim 1, further comprising: receiving biofeedback data from the wearable device comprising a physiological or biochemical state of the user; displaying the biofeedback data to the user as part of a behavioral coaching strategy to improve adoption of healthy behaviors; and adjusting the behavioral coaching program based on the displayed biofeedback data and the user's response.

20. The method of claim 1, further comprising: generating, using a large language model (LLM), an individualized care plan comprising a responsive user interface adapted to capture user-agreed goals and provide follow-up actions; enabling the user to review and modify the one or more sub-goals through the responsive user interface; and updating the individualized care plan iteratively based on the user's input and ongoing wearable device data.
